(19) United States
(12) Patent Application Publication
(10) Pub. No.: US 2010/0016224 A1
(43) Pub. Date: Jan. 21, 2010

(54) COMPOSITIONS AND METHODS FOR MODULATING AN IMMUNE RESPONSE

(75) Inventors: Andrew Bowie, Dublin (IE); Martina Schroeder, Dublin (IE); Geoffrey Smith, London (GB); Stuart Lucas, London (GB)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: The Provost, Fellows and Scholars of the College of the Holy and Undivided Trinity of Queen Elizabe, Dublin (IE)

(21) Appl. No.: 12/438,855

(22) PCT Filed: Aug. 27, 2007
(86) PCT No.: PCT/EP07/58895

§ 371 Date: Feb. 25, 2009

(30) Foreign Application Priority Data
Aug. 25, 2006 (IE) 2006/0638

(51) Int. Cl. A61K 38/16; A61K 31/7052; C12N 7/00; A61P 31/12; A61P 29/00; A61P 37/06

(52) U.S. Cl. 514/12; 514/44 R; 435/235.1

(57) ABSTRACT

The present invention relates to a method of enhancing a pro-inflammatory immune response through the administration of the DEAD-box protein DDX3, as encoded by SEQ ID NO:1. The invention further extends to a method of suppressing an aberrant immune response, such at that associated with autoimmune conditions, through inhibition of DDX3. The invention also extends to a method of suppressing a pro-inflammatory immune response through the administration of the vaccinia virus protein K7. The invention further extends to the provision of an attenuated poxvirus wherein the K7R gene which encodes for the K7 protein is deleted or rendered non-functional. Pharmaceutical compositions comprising DDX3 inhibitory compounds, such as K7, are also provided.

### FIELD OF INVENTION

The present invention relates to compositions and methods for the modulation of the immune system. More specifically, there is provided the identification of a novel function for the DEAD-box protein DDX3. The invention provides methods of enhancing a pro-inflammatory immune response through the administration of DDX3, along with uses of DDX3 in the modulation of an immune response. The invention further extends to methods of suppressing a pro-inflammatory or aberrant immune response through inhibition of DDX3 and to use of DDX3 inhibitors in the suppression of an immune response. The invention further extends to the identification of a novel function ascribed to K7, a vaccinia virus protein conserved in many poxviruses, including variola, the causative agent of smallpox. The invention extends to methods of modulating an immune response through the administration of K7, along with uses of K7 in the modulation of an immune response. The invention further extends to the provision of an attenuated poxvirus which can be used in vaccination, which through the deletion or attenuation of the K7R gene, which encodes for the K7 protein, exhibits reduced viral immune evasion capabilities.

### BACKGROUND TO THE INVENTION

Two classes of pattern recognition receptors (PRRs) play an important role in innate recognition of viruses: Toll-like receptors (TLRs) and RIG-like helicases (RLHs).

Toll-like receptors (TLR) are PRRs that detect pathogen-associated molecular patterns (PAMP) on microbes and in response trigger signalling pathways leading to the activation of the innate immune system. The first step of TLR signalling is mediated by homotypic interactions between the Toll-Interleukin-1-Resistance (TIR) domains of the receptors and the adaptor molecules, MyD88, Mal, TRIF and TRAM. TLR3, TLR7 and 8 and TLR9 constitute a subset of TLRs that are localised in the endosomal compartment and recognise viral nucleic acids. TLR3 responds to dsRNA, TLR7 and TLR8 to viral ssRNA and TLR9 to viral unmethylated CpG dsDNA. TLR2 and TLR4, best known as sensors of bacterial lipopeptides and LPS, respectively, are also activated by certain viral proteins. A major signalling pathway elicited by all TLRs is the activation of NF-κB (NF-kappaB), which is required for the induction of a range of immune-stimulatory and pro-inflammatory cytokines. NF-κB activation is mediated mainly by the adaptor molecule MyD88 (for TLR2 and TLR4 in conjunction with Mal). TLR3, TLR4, TLR7, TLR8 and TLR9 can also activate another class of transcription factors, the Interferon-regulatory factors (IRF) (also known as Interferon response factors), which lead to the induction of type I interferons (IFNs). In particular, TLR3, TLR4, TLR7, TLR8 and TLR9 have been shown to be capable of activating IRF3 and IRF7. Type I IFNs have well described anti-viral properties. IRF activation is mediated by the adaptor TRIF for TLR3 and TLR4 signalling (for TLR4 in conjunction with TRAM) and by a MyD88-dependent pathway for TLR7 and TLR8 and TLR9. IRF7 has been suggested to be the ‘master regulator’ transcription factor for the anti-viral response.

More recently, TLR-independent mechanisms for the recognition of viral RNA and DNA have been discovered. The cytoplasmic DExD/H box RLHs retinoic acid-inducible gene I (RIG-I) and melanoma differentiation associated gene 5 (Mda-5) bind distinct viral dsRNAs and thereby recognise different RNA viruses. This leads to the activation of NF-κB and IRFs mediated by the adaptor molecule MAVS (mitochondrial antiviral signalling, also called Cardif, VISA and IPS-1). An antiviral response may also be induced by cytoplasmic dsDNA, using a receptor that is independent of RIG-I and Mda-5 but shares some of their downstream signalling proteins such as TBK1. The newly identified DNA-dependent activator of IFN-regulatory factors (DAI, also called DLM-1, ZBP1) is a likely candidate for the detection of viruses that produce dsDNA in the host cell cytoplasm.

In a process of co-evolution with the host, viruses have developed sophisticated mechanisms to evade or subvert key aspects of the host anti-viral response. Therefore, the study of viral evasion mechanisms can help to identify novel aspects of the host anti-viral response. For example, the discovery that the hepatitis C virus (HCV) NS3/4a protease cleaves IPS-1 and thereby disrupts downstream signalling, helped to place IPS-1 in the RIG-I and Mda-5-dependent antiviral pathways.

Vaccinia virus (VACV) is a member of the Poxyiridae, a family of large double-stranded DNA viruses that replicate in the cytoplasm and have evolved a multitude of mechanisms to evade the host immune response. VACV seems to target the innate immune response in particular. For instance, VACV encodes the TLR antagonists A46 and A52 and their discovery provided early evidence for an involvement of TLRs in the anti-viral response. A46 and A52 both contribute to virulence and target different aspects of TLR signalling. While A46 contains a TIR domain and targets host TIR-domain containing proteins, such as the TIR adaptors and the TLRs, A52 interacts with the downstream components of TLR signalling TNF receptor associated factor 6 (TRAF6) and interleukin (IL) receptor associated kinase 2 (IRAK2). IRAK2 is a member of the IRAK family of kinases. IRAK1 and 4 relay the signals from the adaptor molecules to mediate activation of TRAF6. IRAK2 can also bind to MyD88 and TRAF6 and is therefore also likely to be involved in this pathway. By targeting IRAK2, A52 can inhibit TLR-induced NF-κB activation and pro-inflammatory cytokine production, while its interaction with TRAF6 mediates p38 MAP kinase activation and the induction of the anti-inflammatory cytokine IL-10. A52 therefore inhibits NF-κB but not IRF activation, while A46R blocks IRF activation and to a lesser extent NF-κB.

An analysis of the VACV genome revealed a family of A46-like proteins, which comprises not only the described TLR antagonist A52, but also B14, C16 and C6. In addition to A46 and A52, VACV expresses other regulators of NF-κB activation, such as K1, N1, and M2. These proteins show differing degrees of conservation in orthopoxviruses. N1 and M2 are predicted to be expressed by at least one strain of all eight orthopoxvirus species for which complete genome sequences are available: VACV, VARV, ectromelia virus (ECTV), camelpox virus (CMLV), horsepox virus, cowpox virus (CPXV), taterapox virus (TATV) and monkeypox virus. A46 is expressed by all these orthopoxvirus species except TATV, and K1 is not expressed in VARV, TATV and CMLV. In contrast, A52 is less well conserved and is encoded by only VACV, HSPV and CPXV.

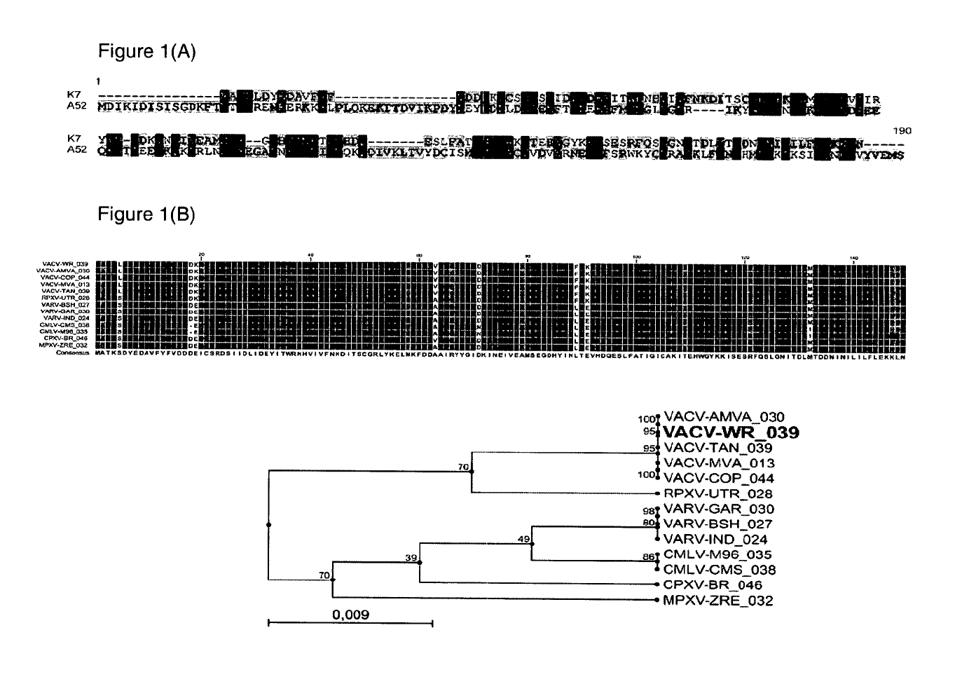

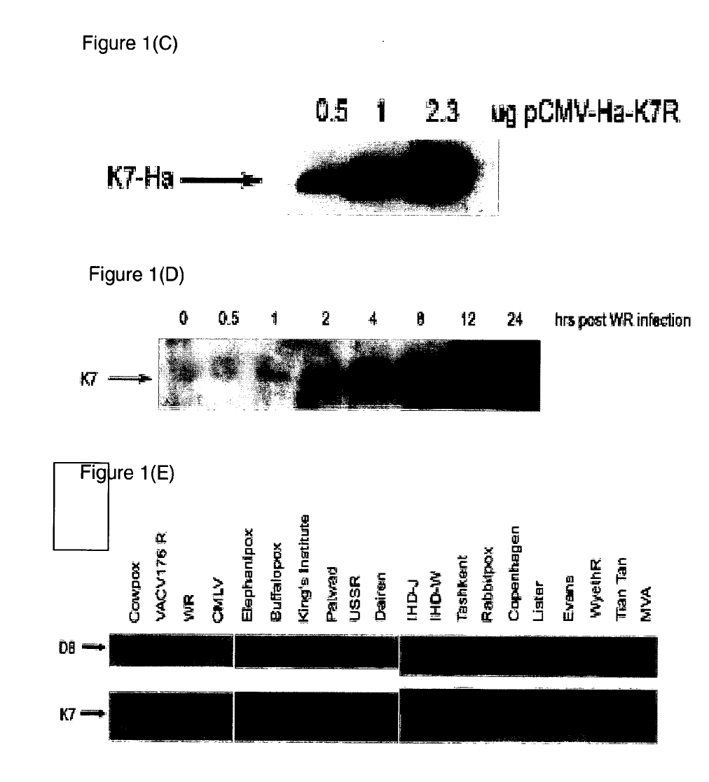

A further VACV protein, termed K7 is related to several of these proteins and shares 25% identity and 50% similarity with A52 (FIG. 1a, see also URL www.poxvirus.org). K7 is also highly conserved and, with the exception of ECTV, is expressed by all orthopoxviruses sequenced (including 48 strains of VARV, 9 strains of monkeypox virus and 14 strains of VACV). However the functions of K7, and its contribution to virulence, are previously unknown.

The DEAD-box protein DDX3 is a putative RNA helicase that is targeted by other viral proteins, namely HCV core protein and HIV Rev. In addition to ATP-dependent RNA helicase activity, DDX3 possesses a nucleocytoplasmic shuttling capacity that is exploited by HIV rev in exporting viral RNAs. Independently, DDX3 was shown to suppress colony formation of tumour cells by upregulating p21^{waf1/cip1} and implicated in the regulation of Cyclin A during G1/S phase transition, demonstrating its diversity of biological functions.

Following extensive experimentation, the present inventors have now surprisingly identified that DDX3 has a role in modulating the immune response, in particular through the activation of the transcription factors IRF and NF-κB. Based on these findings, the inventors have identified the utility of DDX3 in inducing and/or enhancing a pro-inflammatory immune response, said response being desirable in response to infection of a host with a pathogenic organism. Furthermore, the inventors have identified that suppressing and/or blocking DDX3 expression and functional activity can have an important utility in down-regulating an aberrant pro-inflammatory immune response, such as that associated with acute and chronic autoimmune diseases. Accordingly, DDX3 inhibitors or suppressors may have an important utility in the treatment of such diseases.

The present inventors have further surprisingly identified that K7, a vaccinia virus protein conserved in many poxviruses, acts to inhibit the function of the DEAD-box protein DDX3. K7 is therefore identified as having a role in the suppression of the immune response. The elucidation of the function of K7 makes it likely that this protein is employed in subversion of the immune response which is mounted by a subject following infection with vaccinia virus. The inventors have therefore identified that suppressing or blocking the function of K7 can have an important role in preventing suppression of the immune response following infection of a subject with a virus which expresses K7.

More specifically, the surprising observation that K7 has been shown to block cellular signalling pathways which lead to the activation of transcription factors such as NF-κB or IRF can be used to modulate a wide range of immune responses. Further, K7 has been shown to induce the production of the cytokine IL-10, this cytokine having an acknowledged and defined role in the suppression of an immune response. Further still, the deletion of the K7R gene in poxviruses will allow the provision of a poxvirus with reduced immune evasion capabilities, with such a virus being more effective as a vaccine candidate as it would be safer should reversion to virulence of the attenuated virus occur.

The inventors therefore identified that expression of DDX3 leads to enhancement of the activation of the transcription factors NF-κB and IRF. Expression of dominant-negative DDX3 protein in cells mimics the effects seen following administration of the vector encoding K7 protein Furthermore, K7 is shown to be a multifunctional VACV virulence factor that targets DDX3.

### SUMMARY OF THE INVENTION

According to a first aspect of the invention there is provided a method for the treatment and/or prophylaxis of a condition mediated by a pro-inflammatory immune response, the method comprising the step of:
providing a therapeutically effective amount of a compound which inhibits the expression or biological function of a protein comprising the amino acid sequence of SEQ ID NO:1, and
administering the same to a subject in need of such treatment.

In certain embodiments, the protein comprising the amino acid sequence of SEQ ID NO:1 is the DEAD-box protein DDX3.

The amino sequence of the DDX3 protein has been previously defined and is described herein as SEQ ID NO:1 as follows:

MSHVAVENALGLDQQFAGLDLNSSDNQSGGSTASKGRYIPPHLRNREATK
GFYDKDSSGWSSSKDKDAYSSFGSRSDSRGKSSFFSDRGSGSRGRFDDRG
RSDYDGIGSRGDRSGFGKFERGGNSRWCDKSDEDDWSKPLPPSERLEQEL
FSGGNTGINFEKYDDIPVEATGNNCPPHIESFSDVEMGEIIMGNIELTRY
TRPTPVQKHAIPIIKEKRDLMACAQTGSGKTAAFLLPILSQIYSDGPGEA
LRAMKENGRYGRRKQYPISLVLAPTRELAVQIYEEARKFSYRSRVRPCVV
YGGADIGQQIRDLERGCHLLVATPGRLVDMMERGKIGLDFCKYLVLDEAD
RMLDMGFEPQIRRIVEQDTMPPKGVRHTMMFSATFPKEIQMLARDFLDEY
IFLAVGRVGSTSENITQKVVWVEESDKRSFLLDLLNATGKDSLTLVFVET
KKGADSLEDFLYHEGYACTSIHGDRSQRDREEALHQFRSGKSPILVATAV
AARGLDISNVKHVINFDLPSDIEEYVHRIGRTGRVGNLGLATSFFNERNI
NITKDLLDLLVEAKQEVPSWLENMAYEHHYKGSSRGRSKSSRFSGGFGAR
DYRQSSGASSSSFSSSRASSSRSGGGGHGSSRGFGGGGYGGFYNSDG YG
GNYNSQGVDWWGN

The nucleotide sequence of DDX3 that encodes amino acid SEQ ID NO:1 is provided below as SEQ ID NO:2:

atgagtcatgtggcagtggaaaatgcgctcgggctggaccagcagtttgc
tggcctagacctgaactcttcagataatcagagtggaggaagtacagcca
gcaaagggcgctatattcctcctcatttaaggaaccgagaagctactaaa
ggtttctacgataaagacagttcagggtggagttctagcaaagataagga
tgcgtatagcagttttggatctcgtagtgattcaagagggaagtctagct
tcttcagtgatcgtggaagtggatcaaggggaaggtttgatgatcgtgga
cggagtgattacgatggcattggcagccgtggtgacagaagtggctttgg
caaatttgaacgtggtggaaacagtcgctggtgtgacaaatcagatgaag
atgattggtcaaaaccactcccaccaagtgaacgcttggaacaggaactc
ttttctggaggcaacactgggattaattttgagaaatacgatgacattcc
agttgaggcaacaggcaacaactgtcctccacatattgaaagtttcagtg
atgttgagatgggagaaattatcatgggaaacattgagcttactcgttat
actcgcccaactccagtgcaaaagcatgctattcctattatcaaagagaa
aagagacttgatggcttgtgcccaaacagggtctggaaaaactgcagcat
ttctgttgcccatcttgagtcagatttattcagatggtccaggcgaggct
ttgagggccatgaaggaaaatggaaggtatgggcgccgcaaacaataccc
aatctccttggtattagcaccaacgagagagttggcagtacagatctacg
aggaagccagaaaattttcataccgatctagagttcgtccttgcgtggtt
tatggtggtgccgatattggtcagcagattcgagacttggaacgtggatg
ccatttgttagtagccactccaggacgtctagtggatatgatggaaagag
gaaagattggattagacttttgcaaatacttggtgttagatgaagctgat
cggatgttggatatggggtttgagcctcagattcgtagaatagtcgaaca
agatactatgcctccaaagggtgtccgccacactatgatgtttagtgcta
cttttcctaaggaaatacagatgctggctcgtgatttcttagatgaatat
atcttcttggctgtaggaagagttggctctacctctgaaaacatcacaca
gaaagtagtttgggtggaagaatcagacaaacggtcatttctgcttgacc
tcctaaatgcaacaggcaaggattcactgaccttagtgtttgtggagacc
aaaaagggtgcagattctctggaggatttcttataccatgaaggatacgc
atgtaccagcatccatggagaccgttctcagagggatagagaagaggccc
ttcaccagttccgctcaggaaaaagcccaattttagtggctacagcagta
gcagcaagaggactggacatttcaaatgtgaaacatgttatcaattttga
cttgccaagtgatattgaagaatatgtacatcgtattggtcgtacgggac
gtgtaggaaaccttggcctggcaacctcattctttaacgagaggaacata
aatattactaaggatttgttggatcttcttgttgaagctaaacaagaagt
gccgtcttggttagaaaacatggcttatgaacaccactacaagggtagca
gtcgtggacgttctaagagtagcagatttagtggagggtttggtgccaga
gactaccgacaaagtagcggtgccagcagttccagcttcagcagcagccg
cgcaagcagcagccgcagtggcggaggtggccacggtagcagcagaggat
ttggtggaggtggctatggaggcttttacaacagtgatggatatggagga
aattataactcccagggggttgactggtggggtaactga

In certain embodiments, the compound is a polypeptide comprising the amino acid sequence of SEQ ID NO:3 or an analogue, derivative, fragment, variant or peptidomimetic thereof.

The K7 polypeptide as derived from vaccinia virus is defined as having the amino acid sequence of SEQ ID NO:3. The DEAD-box helicase DDX3 has been purported to have a role in the life cycle of HIV. The exact involvement of DEAD-box helicase DDX3 in the HIV life cycle has not yet been defined, but it is known that DEAD-box helicase DDX3 constantly shuttles between the nucleus and the cytoplasm. Without wishing to be bound by theory, the present inventors predict that complexes which are formed between DDX3 and K7, either in the cytoplasm or in the nuclear compartment, can mediate an effect which serves to modulate the immune response by down-regulating the response.

The complex formed between DDX3 and K7 could either (i) result in K7 utilising DDX3 and subverting its function, or (ii) could result in an inhibition of the function of DDX3, with this inhibition suppressing a process which is detrimental to the virus. The complex between K7 and DDX3 may have an inhibitory effect on the HIV life cycle, for example, by preventing the passage of HIV viral components out of the nuclear compartments.

In certain embodiments, the compound is a polynucleotide which encodes a polypeptide having the amino acid sequence of SEQ ID NO:3 or an analogue, derivative, fragment, or variant thereof.

In certain embodiments, the compound is a polynucleotide comprising the sequence of SEQ ID NO:4.

In certain embodiments, the compound is an inhibiting nucleic acid which blocks the functional expression of a DEAD-box protein DDX3 having the amino acid sequence of SEQ ID NO:1. Typically the inhibiting nucleic acid can include, but is not limited to, anti-sense oligonucleotides, anti-sense DNA, anti-sense RNA, ribozymes, iRNA, miRNA, siRNA or shRNA.

As herein defined, the terms “blocks” or “blocking” when used in relation to gene expression means silencing the expression of a gene. Gene silencing is the switching off of the expression of a gene by a mechanism other than genetic modification. Gene silencing can be mediated at the transcriptional or post-transcriptional level. Transcriptional gene silencing can result in a gene being inaccessible to transcriptional machinery, and can be mediated, for example, by means of histone modifications. Post-transcriptional gene silencing results from the mRNA of a gene being destroyed, this preventing an active gene product, such as a protein, in the present case the DEAD-box protein DDX3.

In certain embodiments, the compound which inhibits the expression or biological function of a protein comprising the amino acid sequence of SEQ ID NO:1 is an inhibitory molecule such as an antibody, in particular a monoclonal antibody, or a binding fragment derived from an antibody which has binding specificity for a DEAD-box protein DDX3 having the amino acid sequence of SEQ ID NO:1.

Accordingly, in certain embodiments, the compound is an inhibiting nucleic acid which blocks the functional expression of a DEAD-box protein DDX3 having the amino acid sequence of SEQ ID NO:1, such as an interfering ribonucleic acid (for example an siRNA or shRNA) or a transcription template thereof, such as a DNA encoding an shRNA which mediates blocking of the gene expression relating to the DEAD-box protein DDX3.

In certain embodiments, the inhibitory molecule is antisense RNA. Antisense causes suppression of gene expression and involves single stranded RNA fragments which physically bind to mRNA, thus blocking mRNA translation.

Techniques for the preparation of appropriate nucleic acid for use as inhibiting nucleic acids are well known to the person skilled in the art.

In certain embodiments, the pro-inflammatory immune response which is suppressed using the method of this aspect of the invention is characterised in that it is mediated through the activation of at least one of the transcription factors selected from NF-κB and at least one IRF. Typically, the IRF may suitably be IRF3 or IRF7.

Although NF-κB and the IRFs are key transcription factors used in TLR and IL-1 Receptor mediated signalling, they are also involved in other signalling pathways. Accordingly signalling through NF-κB and the IRFs is likely to contribute to inflammation in different contexts. Accordingly, the present invention has further utility in the blocking or suppression of the function of NF-κB and at least one IRF so as to suppress or down-regulate the immune response, as neither NF-κB nor the IRFs, and in particular IRF3 and IRF7, will transactivate promoters leading to enhancement of gene expression.

In certain embodiments, the condition mediated by a pro-inflammatory immune response is an immune-mediated condition, that is, that the pathology of the condition is associated in whole, or in part, with an aberrant immune response being mounted by a host.

In certain embodiments, the immune-mediated condition is an autoimmune disease or associated autoimmune condition.

Autoimmune diseases and conditions may include, but are not limited to; multiple sclerosis, rheumatoid arthritis, Crohn's disease, psoriasis, systemic lupus erythematosis (SLE), lupus, type I diabetes, colitis, inflammatory bowel disease, asthma and allergy.

In certain further embodiments, the immune-mediated condition may include, but is not limited to: diabetes mellitus, myasthenia gravis, autoimmune thyroiditis, dermatitis (including atopic dermatitis and eczematous dermatitis), Sjogren's Syndrome, including keratoconjunctivitis sicca secondary to Sjogren's Syndrome, alopecia greata, allergic responses due to arthropod bite reactions, aphthous ulcer, iritis, conjunctivitis, keratoconjunctivitis, ulcerative colitis, cutaneous lupus erythematosus, scieroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves opthalmopathy, sarcoidosis, primary biliary cirrhosis, uveitis posterior, interstitial lung fibrosis, Alzheimer's disease and coeliac disease, or atopic disease.

In addition to the above listed autoimmune conditions, the present invention may be extended to any immune mediated disorder where an undesirable or unwanted (aberrant) immune response is triggered by the presentation of antigen(s).

In certain further embodiments, the immune-mediated condition may be a condition characterised by the occurrence of an undesirable immune response. Such conditions include, inter alia, those wherein the immune response is directed to a self antigen as well as to those wherein the immune response may be regarded as being physiologically normal but is nevertheless undesirable.

In certain embodiments, the immune-mediated condition relates to an immune response which results in the rejection of a graft, for example, donor cells, tissue or an organ being transplanted into a host.

According to a second aspect of the invention there is provided a pharmaceutical composition for suppressing a pro-inflammatory immune response, comprising a therapeutically effective amount of a compound which inhibits the function or expression of the DEAD-box protein DDX3 having the amino acid sequence of SEQ ID NO:1 along with a pharmaceutically acceptable diluent, excipient or carrier.

In certain embodiments, the DDX3 inhibitory compound is a polypeptide comprising the amino acid sequence of SEQ ID NO:3 or an analogue, derivative, fragment, variant or peptidomimetic thereof.

In certain embodiments, the DDX3 inhibitory compound is a polynucleotide which encodes a polypeptide having the amino acid sequence of SEQ ID NO:3 or an analogue, derivative, fragment, or variant thereof.

In certain embodiments, the DDX3 inhibitory compound is a polynucleotide comprising the sequence of SEQ ID NO:4.

In certain embodiments, the DDX3 inhibitory compound is an inhibiting nucleic acid which blocks the functional expression of a DEAD-box protein DDX3 having the amino acid sequence of SEQ ID NO:1. Typically the inhibiting nucleic acid can include, but is not limited to, anti-sense oligonucleotides, anti-sense DNA, anti-sense RNA, ribozymes, iRNA, miRNA, siRNA or shRNA.

In certain embodiments, the DDX3 inhibitory is an inhibitory molecule such as an antibody, in particular a monoclonal antibody, or a binding fragment derived from an antibody which has binding specificity for a DEAD-box protein DDX3 having the amino acid sequence of SEQ ID NO:1.

In certain embodiments, the pro-inflammatory immune response which is suppressed using the pharmaceutical composition of this aspect of the invention is characterised in that it is mediated through the activation of at least one of the transcription factors selected from NF-κB and at least one IRF. Typically, the IRF may suitably be IRF3 or IRF7.

According to a third aspect of the present invention, there is provided the use of a compound which inhibits the biological function or blocks the expression of the DEAD-box protein DDX3 having the amino acid sequence of SEQ ID NO:1 for administration to a subject for the suppression of a pro-inflammatory immune response.

In certain embodiments, the suppression of the pro-inflammatory immune response results from inhibition of DDX3 functional activity preventing the activation of at least one transcription factor. Typically, said transcription factors are selected from the group comprising, but not limited to NF-κB, or at least one IRF (interferon response element). In certain embodiments, the IRF may be IRF3 and/or IRF7.

According to a fourth aspect of the present invention, there is provided the use of a compound which inhibits the biological function or blocks the expression of the DEAD-box protein DDX3 having the amino acid sequence of SEQ ID NO:1 in the preparation of a medicament for the suppression of a pro-inflammatory immune response.

In certain embodiments, the suppression of the pro-inflammatory immune response results from inhibition of DDX3 functional activity preventing the activation of at least one transcription factor. Typically, said transcription factors are selected from the group comprising, but not limited to NF-κB, or at least one IRF (interferon response element).

In certain embodiments, the pro-inflammatory immune response which is suppressed is mediated through the activation of at least one of the transcription factors selected from NF-κB and at least one IRF. Typically, the IRF may suitably be IRF3 and/or IRF7.

The observation by the inventors that the DEAD-box protein DDX3 has a pivotal role in the signalling pathway which results in a pro-inflammatory immune response being mediated has led the inventors to identify a further utility for DDX3 in relation to its administration in order to mediate or enhance a pro-inflammatory immune response. There are a number of instances where it is desirable to mediate a pro-inflammatory immune response in a host, for example, in order to provide protective immunity against a viral infection or other pathogenic organism which is infecting a subject.

Accordingly a fifth aspect of the invention provides a method for mediating or promoting a pro-inflammatory immune response, the method comprising the steps of:
providing a composition comprising a therapeutically effective amount of a DEAD-box protein DDX3 having the amino acid sequence of SEQ ID NO:1 or an analogue, derivative, fragment, variant or peptidomimetic thereof, and
administering the same to a subject in need of said treatment.

In certain embodiments, the pro-inflammatory immune response which is mediated or enhanced using the method of this aspect of the invention is characterised in that it is mediated through the activation of at least one of the transcription factors selected from NF-κB and at least one IRF. Typically, the IRF may suitably be IRF3 and/or IRF7.

According to a sixth aspect of the present invention there is provided a pharmaceutical composition for use in mediating a pro-inflammatory immune response, said composition comprising a therapeutically effective amount of a DEAD-box protein DDX3 having the amino acid sequence of SEQ ID NO:1 or an analogue, derivative, fragment, variant or peptidomimetic thereof, and at least one pharmaceutically acceptable diluent, excipient or carrier.

According to a seventh aspect of the present invention there is provided the use of a DEAD-box protein DDX3 having the amino acid sequence of SEQ ID NO:1 in the preparation of a medicament for mediating a pro-inflammatory immune response in a subject.

According to an eighth aspect of the present invention there is provided the use of a polynucleotide which encodes a DEAD-box protein DDX3 having the amino acid sequence of SEQ ID NO:1 in the preparation of a medicament for mediating a pro-inflammatory immune response in a subject.

In certain embodiments, the polynucleotide has a nucleic acid sequence as defined in SEQ ID NO:2.

The present invention has further utility in suppressing or down-regulating immune responses which are mediated by the intracellular signalling pathways activated following the binding of Toll-like Receptors by their respective ligands.

Accordingly, a further aspect of the present invention provides a method of suppressing an intracellular signalling pathway induced by an activated Toll-like Receptor, wherein said signalling pathway activates at least one transcription factor, said method comprising the steps of:
providing a therapeutically effective amount of a compound which inhibits the expression or biological function of a DEAD-box protein DDX3 having the amino acid sequence of SEQ ID NO:1, and
administering the same to a subject in need of such treatment.

In certain embodiments, the Toll-like Receptor is activated following the binding of a pathogen-associated molecular pattern (PAMP) to the Toll-like Receptor.

In certain embodiments, the transcription factor may be NF-κB and/or at least one IRF. Typically the IRF is IRF3 or IRF7.

In certain embodiments, the DDX3 inhibitory compound is the K7 polypeptide comprising the amino acid sequence of SEQ ID NO:3 or an analogue, derivative, fragment, variant or peptidomimetic thereof.

In certain embodiments, the DDX3 inhibitory compound is a polynucleotide which encodes a polypeptide having the amino acid sequence of SEQ ID NO:3 or an analogue, derivative, fragment, or variant thereof.

In certain embodiments, the DDX3 inhibitory compound is a polynucleotide comprising the sequence of SEQ ID NO:4.

In certain embodiments, the DDX3 inhibitory compound is an inhibiting nucleic acid which blocks the functional expression of a DEAD-box protein DDX3 having the amino acid sequence of SEQ ID NO:1. Typically the inhibiting nucleic acid can include, but is not limited to, anti-sense oligonucleotides, anti-sense DNA, anti-sense RNA, ribozymes, iRNA, miRNA, siRNA or shRNA.

In certain embodiments, the DDX3 inhibitory is an inhibitory molecule such as an antibody, in particular a monoclonal antibody, or a binding fragment derived from an antibody which has binding specificity for a DEAD-box protein DDX3 having the amino acid sequence of SEQ ID NO:1.

In certain embodiments, the pro-inflammatory immune response which is suppressed using the pharmaceutical composition of this aspect of the invention is characterised in that it is mediated through the activation of at least one of the transcription factors selected from NF-κB and at least one IRF. Typically, the IRF may suitably be IRF3 or IRF7.

Although the K7 polypeptide has been identified by the inventors as mediating a suppressing effect on DDX3, it is known that the DEAD-box protein DDX3 is a putative RNA helicase that is targeted by other viral proteins, namely HCV core protein and HIV Rev. However, the present inventors recognise the utility of identifying further inhibitors of DDX3 function. Such inhibitory compounds may include: proteins, peptides, peptidomimetics, nucleic acids, polynucleotides, polysaccharides, oligopeptides, carbohydrates, lipids, small molecule compounds and naturally occurring compounds. Compounds or molecules which serve to suppress or inhibit the function of DDX3 would have particular utility when administered to individuals suffering from a disease or condition caused by an aberrant immune response.

Accordingly, the invention extends to screening assay methods, and to substances identified thereby which have utility in inhibiting or blocking the function of DDX3.

A further aspect of the present invention provides for the use of the DDX3 protein (including a fragment or derivative thereof) in screening or searching or obtaining or identifying a substance, such as a peptide or a chemical compound, which interacts with or binds to DDX3 in order to suppress or inhibit its biological function.

For instance, a method according to one aspect of the present invention includes providing DDX3 or a variant or a fragment thereof and bringing this into contact with a substance, which contact may result in binding between DDX3 and the substance. Binding may be determined by any number of techniques, both qualitative and quantitative, which would be known to the person skilled in the art.

As such, a further aspect of the present invention extends to a screening method for at least one modulator which inhibits, downregulates, blocks or suppresses the functional activity or the expression of the DEAD-box protein DDX3 having the amino acid sequence of SEQ ID NO:1, the method comprising the steps of:
(i) providing first and second cellular samples containing the DEAD-box protein DDX3 having the amino acid sequence of SEQ ID NO:1 or an analogue, derivative, fragment, variant or peptidomimetic thereof,
(ii) contacting said first sample with a candidate modulator of the DDX3 protein or an analogue, derivative, fragment, variant or peptidomimetic thereof,
(iii) contacting said first and second samples with a Toll-like Receptor agonist or other suitable modulator of the immune response, and
(iv) monitoring the level of an immune mediator produced as part of the signalling pathway which is causative of an immune response, said mediator preferably being at least one of the transcription factors NF-κB or an IRF, and comparing the level of said mediators between said first and second samples,
   wherein a difference in the level of the mediators between said first and second samples identifies the candidate modulator as a modulator of DDX3 activity.

In certain embodiments, the IRF may be IRF3 and/or IRF7.

In a further embodiment the mediator of immune function and signalling which is used to monitor immune activity is RIG-1.

The modulator(s) identified according to the above assays of this aspect of the present invention may be a peptide or non-peptide molecule such as a chemical entity or pharmaceutical substance. Where the modulator is a peptide it may be an antibody, an antibody fragment, or a similar molecule with binding activity. Further, where the modulator is an antibody, it is preferably a monoclonal antibody.

A further aspect of the present invention provides for the use of a modulator identified according to the previous aspect of the invention in the preparation of a medicament for modulating the signalling mediated through a Toll-like Receptor.

A further still aspect of the present invention provides for the use of a modulatory compound identified according to the previous aspect of the invention in the preparation of a medicament for modulating the signalling mediated by the transcription factor NF-κB and/or IRF.

A substance identified as a modulator of DDX3 function may be a peptide or non-peptide in nature. Non-peptide “small molecules” are often preferred for many in-vivo pharmaceutical uses. Accordingly, a mimetic or mimic of the substance may be designed for pharmaceutical uses. The designing of mimetics to a known pharmaceutically active compound is a known approach to the development of pharmaceuticals based on a “lead” compound. This might be desirable where the active compound is difficult or expensive to synthesise or where it is unsuitable for a particular method of administration, e.g. peptides are not well suited as active agents for oral compositions as they tend to be quickly degraded by proteases in the alimentary canal. Mimetic design, synthesis and testing may be used to avoid randomly screening large number of molecules for a target property.

A further aspect of the present invention therefore provides an assay for assessing binding activity between a DDX3 peptide and a putative binding molecule which includes the steps of:
bringing DDX3 or a variant or fragment thereof into contact with a putative binding molecule or other test substance, and
determining interaction or binding between DDX3 and the binding molecule or test surface.

A substance which interacts with DDX3 may be isolated and/or purified, manufactured and/or used to modulate the activity of DDX3.

It is not necessary to use the entire DDX3 protein for assays which test for binding between two molecules. Fragments may be generated and used in any suitable way known to the person skilled in the art.

Further, the precise format of the assay of the invention may be varied by those skilled in the art using routine skill and knowledge.

In a further aspect of the present invention there is provided a method of modulating intracellular signalling mediated by IL-1R or Toll-like Receptors, the method comprising the steps of:
providing a therapeutically effective amount of a compound which inhibits the expression or biological function of a DEAD-box protein DDX3 having the amino acid sequence of SEQ ID NO:1, and
administering the same to a subject in need of such treatment.

A further aspect of the present invention provides for the use of a peptide, or an analogue, derivative, fragment, variant or peptidomimetic thereof, which inhibits or downregulates DDX3 in the modulation of intracellular signalling mediated by IL-1R or Toll-like Receptors following the binding of a suitable agonist.

A further aspect of the present invention provides a method of suppressing the production of interferon mediated by TLR or RIG-I signalling pathways, the method comprising the step of administering a peptide, or an analogue, derivative, fragment, variant or peptidomimetic thereof, which inhibits or downregulates DDX3 to an individual in need of such treatment.

IL-10 has been identified as being a potent modulator of the immune response. Specifically, IL-10 has been identified as having an important role as a key anti-inflammatory and immunoregulatory cytokine. IL-10 has, for example, an identified role in activating dendritic cells into a phenotype that promotes the production of regulatory T cells (Tregs), these Tregs in turn modulating the immune response, through the suppression of Th1 and ThIL-17 type responses.

According to a further aspect of the present invention there is provided a pharmaceutical composition for the prevention and/or treatment of a T cell mediated inflammatory immune response, said composition comprising a peptide, or an analogue, derivative, fragment, variant or peptidomimetic thereof, which inhibits or downregulates DDX3.

According to a yet further aspect of the present invention there is provided a method for modulating a T cell mediated immune response in a subject, the method comprising the step of;
providing a therapeutically effective amount of a compound which inhibits the expression or biological function of a DEAD-box protein DDX3 having the amino acid sequence of SEQ ID NO:1, and
administering the same to a subject in need of such treatment.

In certain embodiments of this aspect of the invention the T cell mediated immune response is suppressed. This suppression may result from a step of contacting an immune cell with an agent comprising a peptide, or an analogue, derivative, fragment, variant or peptidomimetic thereof, which inhibits or downregulates DDX3, in accordance with the method of this aspect of the invention.

A further embodiment of the invention provides for the effective amount of the agent comprising a peptide, or an analogue, derivative, fragment, variant or peptidomimetic thereof, which inhibits or downregulates DDX3 to couple, bind or otherwise associate with a cell surface activation molecule of at least one type of immune cell, this resulting in the suppression, inhibition or down-regulation of one or more functional activities of that cell.

In certain embodiments of the invention, the immune cell whose function is modulated is at least one cell of the innate immune system.

In a further embodiment the cell is a cell type with antigen processing and presenting function, such as an antigen presenting cell (APC), for example a dendritic cell, or a macrophage or a B cell.

Where the APC is a dendritic cell it may be an immature dendritic cell, a semi-mature dendritic cell or it may be a mature dendritic cell.

In a further embodiment the cell of the innate immune system is a cell which does not function as an antigen presenting cell, for example a mast cell. Mast cells secrete cytokines such IL-4 and are accordingly known to have a role in facilitating the immune response, however they do not have an associated antigen processing function.

In certain embodiments, the subject is a mammal. In a further embodiment, the mammal is a human.

A yet further aspect of the present invention provides a method of inducing the production of the cytokine IL-10 by the cells of the immune system, the method comprising the step of:
providing a therapeutically effective amount of a compound which inhibits the expression or biological function of a DEAD-box protein DDX3 having the amino acid sequence of SEQ ID NO:1, and
administering the same to a subject in need of such treatment.

In individuals infected with a virus, such as a poxvirus, for example variola (which is causative of smallpox), DDX3 may be inhibited or down-regulated as part of the virus' immune evasion system. The administration of DDX3 may therefore provide a valuable therapy which could be provided to prevent or limit immune evasion caused by suppression of DDX3, thus enhancing the anti-viral immune response mounted by an infected host to virus infection.

A further aspect of the present invention therefore provides a method of prophylaxis and/or treatment of a viral condition, the methods comprising the step of administering an agent comprising a therapeutically effective amount of a peptide having SEQ ID NO:1 or an analogue, derivative, fragment, variant or peptidomimetic thereof to an individual in need of such treatment wherein the administration of the agent serves to cause activation of at least one of the transcription factor NF-κB and at least one IRF.

In certain embodiments, the viral condition is any viral condition mediated at least in part by suppression of DDX3.

In certain embodiments, the viral condition is caused by a poxvirus, such as variola.

A further aspect of the present invention provides a method of modulating a Toll-like Receptor induced intracellular signalling pathway which includes activation of NF-κB and/or at least one IRF, said method comprising the step of administering a therapeutically effective amount of a peptide having SEQ ID NO:1 or an analogue, derivative, fragment, variant or peptidomimetic thereof to an individual whom requires such a treatment.

A further aspect of the present invention relates to a method of activating or upregulating a signalling function of NF-κB and/or at least one IRF, said method comprising the step of administering a therapeutically effective amount of a peptide having SEQ ID NO:1 or an analogue, derivative, fragment, variant or peptidomimetic thereof to an individual whom requires such a treatment.

A yet further aspect of the present invention provides for use of a peptide having SEQ ID NO:1 or an analogue, derivative, fragment, variant or peptidomimetic thereof in activating or upregulating a signalling function of NF-κB and/or IRF.

In one aspect of the present invention, there is provided a method of upregulating the production of a type I interferon mediated by Toll-like Receptor signalling pathways, the method comprising the step of administering a peptide having SEQ ID NO:1 or an analogue, derivative, fragment, variant or peptidomimetic thereof to an individual in need of such treatment.

A further aspect of the present invention relates to a method for modulating a T cell mediated immune response in a subject, the method comprising the step of administering an effective amount of an agent comprising a peptide having SEQ ID NO:1 or an analogue, derivative, fragment, variant or peptidomimetic thereof to an individual in need of such treatment.

A further aspect of the present invention relates to a method of suppressing the production of cytokine IL-10 by cells of the immune system, the method comprising the step of administering an effective amount of an agent comprising a peptide having SEQ ID NO:1 or an analogue, derivative, fragment, variant or peptidomimetic thereof to an individual in need of such treatment.

The invention further extends to the identification by the inventors of a novel vaccinia virus protein which has been termed K7. The defined amino acid sequence of K7 is provided herein as SEQ ID NO:3. The defined polynucleotide sequence which encodes the K7 polypeptide of SEQ ID NO:3 is provided herein as SEQ ID NO:4. The present inventors have recognized the utility of the K7 protein in methods for the suppression of the pro-inflammatory immune response. The mechanisms which are employed by K7 in order to mediate immune evasion of a virus which is infecting a host cell can be used to suppress or downregulate an aberrant immune response, such as an immune response associated with an autoimmune disease.

The inventors have surprisingly identified that K7 is an inhibitor if the DEAD-box protein DDX3. Furthermore, K7 may target further mediators which are involved in the signalling pathway which results in a pro-inflammatory immune response being mediated.

Accordingly, a further aspect of the invention relates to a polypeptide having the amino acid sequence of SEQ ID NO:3 or an analogue, derivative, fragment, variant or peptidomimetic thereof.

Accordingly, a yet further aspect of the invention provides for a method of suppressing a pro-inflammatory immune response, the method comprising the steps of:
providing a therapeutically effective amount of the K7 protein having the amino acid sequence of SEQ ID NO:3 or an analogue, derivative, fragment, variant or peptidomimetic thereof, and
administering the same to a subject in need of such therapy.

The amino sequence of the K7 (VACV_WR039) protein, which has been previously defined, is described herein as SEQ ID NO:3 as follows:

MATKLDYEDAVFYFVDDDKICSRDSIIDLIDEYITWRNHVIVFNKDITSC
GRLYKELMKFDDVAIRYYGIDKINEIVEAMSEGDHYINFTKVHDQESLFA
TIGICAKITEHWGYKKISESRFQSLGNITDLMTDDNINILILFLEKKLN

The nucleotide sequence of the K7 (VACV_WR039) protein is provided below as SEQ ID NO:4:

atggcgacta aattagatta tgaggatgct gttttttact
ttgtggatga tgataaaata tgtagtcgcg actccatcat
cgatctaata gatgaatata ttacgtggag aaatcatgtt
atagtgttta acaaagatat taccagttgt ggaagactgt
acaaggaatt gatgaagttc gatgatgtcg ctatacggta
ctatggtatt gataaaatta atgagattgt cgaagctatg
agcgaaggag accactacat caattttaca aaagtccatg
atcaggaaag tttattcgct accataggaa tatgtgctaa
aatcactgaa cattggggat acaaaaagat ttcagaatct
agattccaat cattgggaaa cattacagat ctgatgaccg
acgataatat aaacatcttg atactttttc tagaaaaaaa
attgaattga

In certain embodiments, the invention extends to amino acid sequences which have a sequence homology of at least 80% identity when aligned with the amino acid sequence of SEQ ID NO:3. In further embodiments such sequences may have at least 85%, 90%, 95%, 96%, 97%, 98%, 99% or 99.5% amino acid sequence homology when aligned against the amino acid sequence of SEQ ID NO:3.

Accordingly, a yet further aspect of the invention provides for a method of suppressing a pro-inflammatory immune response, the method comprising the steps of:
providing a therapeutically effective amount of a polynucleotide which encodes a polypeptide having the amino acid sequence of SEQ ID NO:3 or an analogue, derivative, fragment, variant or peptidomimetic thereof, and
administering the same to a subject in need of such therapy.

In one embodiment the immune response is mediated through the activation of at least one of the transcription factors selected from NF-κB and at least one IRF. The IRF may suitably be IRF3 or IRF7.

A further aspect of the present invention provides the use of a polynucleotide or a fragment thereof which encodes a protein having SEQ ID NO:3 for the administration to a subject for the suppression of a pro-inflammatory immune response, said immune response being characterised in that it is mediated through the activation of at least one of the transcription factors selected from NF-κB and at least one IRF. The IRF may suitably be IRF3 or IRF7.

A yet further aspect of the present invention provides a pharmaceutical composition comprising a therapeutically effective amount of a polynucleotide or a fragment thereof which encodes a protein having the amino acid sequence of SEQ ID NO:3 along with a pharmaceutically acceptable diluent, excipient or carrier.

A yet further aspect of the present invention provides for the use of a peptide having an amino acid sequence as defined in SEQ ID NO:3 or an analogue, derivative, fragment, variant or peptidomimetic thereof in the preparation of a medicament for the treatment of an immune mediated condition.

Without wishing to be bound by theory, the inventors predict that the administration of the peptide having the amino acid sequence of SEQ ID NO:3 causes suppression of the pro-inflammatory immune response by a number of mechanisms which serve, both individually and in combination, to suppress the immune response.

A yet further aspect of the present invention provides the use of a polynucleotide or a fragment thereof which encodes a protein having SEQ ID NO:3 in the preparation of a medicament for the downregulation of a pro-inflammatory immune response.

The K7 protein (the primary amino acid sequence of which is defined in SEQ ID NO:3) has been identified as interacting with and modulating TRAF6 and IRAK2 and further inhibits Toll-like Receptor (TLR) signalling which is mediated through the transcription factor NF-κB. K7 has a further mechanism of action in the inhibition of TNF and RIG-1 mediated NF-κB activation. Further, K7 can inhibit the induction of type I interferons through the suppression of IRFs (interferon response factors), in particular IRF3 and IRF7. K7 has further been shown to mediate the production of the cytokine IL-10 from cells of the immune system, with IL-10 having an acknowledged role as a key anti-inflammatory and immunoregulatory cytokine.

A still further aspect of the present invention provides the use of a peptide comprising the amino acid sequence of SEQ ID NO:3 or an analogue, derivative, fragment, variant or peptidomimetic thereof for the administration to an individual for the suppression of a pro-inflammatory immune response, said immune response being characterised in that it is mediated through the activation of at least one of the transcription factors selected from NF-κB and at least one IRF. The IRF may suitably be IRF3 or IRF7.

In one embodiment, the peptide serves to suppress a pro-inflammatory immune response by inhibiting Toll-like Receptor (TLR) mediated signalling through NF-κB. The peptide may further suppress the immune response by interacting with TRAF6 and IRAK2.

In a yet further embodiment the peptide(s) of this aspect of the invention mediates the suppression of the pro-inflammatory immune response through the inhibition of signalling mediated by RIG-1 (retinoic-acid inducible gene 1).

In a yet further embodiment the peptide effects its mode of action by interacting with the DEAD-box helicase DDX3.

The present inventors have further recognised that the identification of the function of K7 can have substantial utility in relation to identifying compounds which can serve to block, inhibit or suppress the function of K7. Compounds or molecules which serve to suppress or inhibit the function of K7 would have particular utility when administered to individuals who were infected with a virus, such as a poxvirus, for example variola (which is causative of smallpox), where the K7 protein was expressed as part of the virus' immune system evasion mechanism. The blocking or suppression of the function of K7 would therefore provide a valuable therapy which could be provided to prevent or limit immune evasion caused by K7, thus enhancing the anti-viral immune response mounted by the infected host to virus infection.

Accordingly in various further aspects, the present invention further extends to the identification of compounds which act to inhibit, block or suppress the function of K7 or an analogue, derivative, fragment, variant or peptidomimetic thereof.

In particular, further aspects of the present invention relate to screening and assay methods, and to substances identified thereby which have utility in inhibiting or blocking the function of K7.

A further aspect of the present invention provides the use of the K7 peptide (including a fragment or derivative thereof) in screening or searching or obtaining or identifying a substance, such as a peptide or a chemical compound, which interacts with or binds to K7 in order to suppress or inhibit its biological function.

For instance, a method according to one aspect of the present invention includes providing K7 or a variant or a fragment thereof and bringing this into contact with a substance, which contact may result in binding between K7 and the substance. Binding may be determined by any number of techniques, both qualitative and quantitative, which would be known to the person skilled in the art.

Accordingly a further aspect of the invention provides a method for the identification of at least one modulator of K7 activity, said method comprising the steps of:
(i) providing first and second cellular samples containing the K7 protein (a peptide comprising the amino acid sequence of SEQ ID NO:3) or an analogue, derivative, fragment, variant or peptidomimetic thereof,
(ii) contacting said first sample with a candidate modulator of the K7 protein or an analogue, derivative, fragment, variant or peptidomimetic thereof,
(iii) contacting said first and second samples with a Toll-like Receptor agonist or other suitable modulator of the immune response, and
(iv) monitoring the level of an immune mediator produced as part of the signalling pathway which is causative of an immune response, said mediator preferably being at least one of the transcription factors NF-κB or an IRF, and comparing the level of said mediators between said first and second samples,
   wherein a difference in the level of the mediators between said first and second samples identifies the candidate modulator as a modulator of K7 activity.

In one embodiment, the IRF may be IRF3 and/or IRF7.

In a further embodiment the mediator of immune function and signalling which is used to monitor immune activity is RIG-1.

The modulator(s) identified according to the above assays of this aspect of the present invention may be a peptide or non-peptide molecule such as a chemical entity or pharmaceutical substance. Where the modulator is a peptide it may be an antibody, an antibody fragment, or a similar molecule with binding activity. Further, where the modulator is an antibody, it is preferably a monoclonal antibody.

A further aspect of the present invention provides for the use of a modulator identified according to the previous aspect of the invention in the preparation of a medicament for modulating the signalling mediated through a Toll-like Receptor.

A further still aspect of the present invention provides for the use of a modulatory compound identified according to the previous aspect of the invention in the preparation of a medicament for modulating the signalling mediated by the transcription factor NF-κB.

A substance identified as a modulator of K7 function may be a peptide or non-peptide in nature.

A further aspect of the present invention provides an assay for assessing binding activity between a K7 peptide and a putative binding molecule which includes the steps of:
bringing K7 or a variant or fragment thereof into contact with a putative binding molecule or other test substance, and
determining interaction or binding between K7 and the binding molecule or test surface.

A substance which interacts with K7 may be isolated and/or purified, manufactured and/or used to modulate the activity of K7.

It is not necessary to use the entire K7 protein for assays which test for binding between two molecules. Fragments may be generated and used in any suitable way known to the person skilled in the art.

Further, the precise format of the assay of the invention may be varied by those skilled in the art using routine skill and knowledge.

Accordingly in a further aspect of the present invention there is provided a method of modulating intracellular signalling mediated by IL-1R or Toll-like Receptors, the method comprising the step of administering K7 or a derivative, mutant, fragment, variant or peptide thereof to an individual in need of such treatment.

A further aspect of the present invention provides for the use of K7 or a variant, derivative or fragment thereof in the modulation of intracellular signalling mediated by IL-1R or Toll-like Receptors following the binding of a suitable agonist.

A yet further aspect of the present invention provides for the use of K7 or a variant, derivative or fragment thereof in the preparation of a medicament for the modulation of intracellular signalling mediated by IL-1R or Toll-like Receptors following the binding of a suitable agonist.

The intracellular signalling pathway which is activated following the binding of a Toll-like Receptor results in the activation of the transcription factor NF-κB. The translocation of NF-κB into the nucleus results in the expression of a number of cytokines and chemokines which drive and modulate the immune response. While NF-κB is activated following the activation of any TLR (with this activation generally being mediated by the MyD88-dependent signalling pathway), TRIF additionally activates a second transcription factor, IRF3 (interferon regulatory factor 3). IRF3 activation results in interferon production and in particular the expression of the type I interferon, interferon beta (IFN-β).

Interferons have specific utility as part of immune responses which are directed against pathogens of viral origin. However, in some instances, it may be desirable to downregulate or block the expression of interferons, and accordingly the inventors have identified that the administration of K7 can mediate a suppression of the TLR-TRIF signalling pathway and hence, in turn, the expression of interferons. Modulating the immune response in such a way would have particular utility where it is desirable to suppress the interferon response, without suppressing the MyD88-dependent pro-inflammatory cytokine response.

Accordingly, a further aspect of the present invention provides a method of suppressing the production of interferon mediated by TLR signalling pathways which is mediated by the adaptor protein TRIF, the method comprising the step of administering K7 or a derivative, mutant, fragment, variant or peptide thereof to an individual in need of such treatment.

In one embodiment of this aspect of the invention, the method suppresses the expression of interferon following the binding of at least one of TLR3, TLR4, TLR7, TLR8 or TLR9.

In a further embodiment of this aspect of the invention, K7 serves to suppress or inhibit the function of an IRF, in particular IRF3 and/or IRF7.

In one embodiment the interferon is a type I interferon. In a further embodiment, the interferon is interferon beta.

Modulation of the response and cytokine profile expressed by a specific cell type of the immune system can lead, in turn, to a wider modulation of the overall immune response. The downregulation or suppression of interferon production following TLR3 induced TRIF-dependent signalling can therefore cause the modulation of wider immune responses.

In a further aspect of the present invention there is provided a compound which serves to inhibit at least one of the immuno-suppressive functions of the K7 protein for use in enhancing the anti-viral immune response mediated by a host infected with a poxvirus which expresses the K7R gene and hence the K7 peptide.

In one embodiment, the poxvirus is an orthopoxvirus or a derivative thereof. In a further embodiment, the poxvirus is a vaccinia virus, including strains such as Modified Vaccinia Ankara, Tian Tan, Western Reserve, Lister, Acambis 3000 Modified Vaccinia Ankara, LC16m0, 3737, Copenhagen, LC16m8, Lister, Copenhagen, Wyeth, New York City Board of Health, LIVP, Tashkent, King Institute, Praha Virus, 1HD-W, Patwadanger, LC16m0, Bern or Evans. In yet further embodiments, the poxvirus may be Variola Major, Variola Minor, Rabbitpox, Cowpox, Camelpox, Monkeypox, Yaba-Like Disease Virus, Buffalopox and Elephantpox.

In a yet further embodiment, the poxvirus is an orthopoxvirus or a derivative thereof. As herein defined, a ‘derivative’ of a particular virus means any virus that is derived from a particular virus. A derivative may be obtained by repeated passaging of the particular virus. Alternatively, a derivative may be obtained by site directed or random mutagenesis of the particular virus. A derivative generally retains most of the phenotype and genotype of the virus from which it is derived. Typically, the genome has at least 90% or above sequence identity with the genome of the virus from which it is derived.

In further embodiments, the poxvirus may be derived from a parapoxvirus, an avipoxvirus or a yatapoxvirus.

It is known that in order to induce immunity against a pathogen, a vaccine composition may be prepared which induces an immune response in an individual against an antigen which is representative of the pathogen against which protection is desired. One effective way of inducing protection against a pathogen is to administer to an individual an attenuated version of the pathogen. Attenuation causes the pathogen to be administered in a form which will not cause serious disease, but which will provide the immune system with antigenic targets against which an immune response can be generated.

When an attenuated pathogen is administered to an individual, there is a small chance that the attenuated form of the pathogen will revert to its fully infectious form. Where the pathogen is a virus, this is known as reversion to virulence or reversion to a virulent phenotype.

The administration of poxviruses as attenuated viruses is commonly performed, particularly against viral pathogens such as smallpox. The present inventors have recognised the utility of the present invention in the preparation of an improved attenuated version of a poxvirus for use in vaccination. In particular, the inventors have identified that mutation or deletion of the K7R gene encoding for the K7 protein can prevent this protein being expressed by the virus, thus disabling one important immune evasion mechanism of the viral pathogen. Should reversion to virulence occur, the absence of K7 would strongly weaken the immune evasion capabilities of the virus. Hence, this strategy can be used to formulate improved, safer attenuated viruses.

Accordingly, a yet further aspect of the present invention provides a method of attenuating the virulence of a poxvirus, the method comprising the steps of:
suppressing or partially suppressing the expression of the K7R gene product.

In certain embodiments, the suppression of the gene is performed by mutation or deletion of the gene, and in particular the deletion of a whole or part, or the mutation of a whole or part of the nucleotide sequence encoding K7 from the viral genome.

In certain embodiments, the suppression of the gene is performed by the administration or inhibitory nucleotides which act at the nucleic acid level through the use of techniques which will be well known to the person skilled in the art, for example through the use of antisense or siRNA's and other suppression effectors such as nucleic acids and ribozymes, triple helix forming oligonucleotides and peptides and/or antibodies or antibody-like binding fragments directed to the K7R gene sequence or transcripts or protein.

Suitably, the attenuated orthopoxvirus prepared according to the method of this aspect of the invention may be administered as an attenuated vaccine to an individual in need of such treatment.

Accordingly a further aspect of the present invention provides for the use of an attenuated orthopoxvirus prepared in accordance with the method of the previous aspect of the invention as the immunogenic determinant in a vaccine composition for inducing long term protective immunity against the orthopoxvirus.

In one embodiment, the poxvirus is an orthopoxvirus or a derivative thereof. In a further embodiment, the recombinant poxvirus is a vaccinia virus, including strains such as Modified Vaccinia Ankara, Tian Tan, Western Reserve, Lister, Acambis 3000 Modified Vaccinia Ankara, LC16m0, 3737, Copenhagen, LC16m8, Lister, Copenhagen, Wyeth, New York City Board of Health, LIVP, Tashkent, King Institute, Praha Virus, IHD-W, Patwadanger, LC16m0, Bern or Evans. In yet further embodiments, the poxvirus may be Variola Major, Variola Minor, Rabbitpox, Cowpox, Camelpox, Monkeypox, Yaba-Like Disease Virus, Buffalopox and Elephantpox.

In a further aspect, the present invention extends to recombinant poxviruses which have improved properties as vaccines. Accordingly the present invention provides a recombinant poxvirus, wherein the genome has been modified such that it does not encode for a functional K7R gene.

In one embodiment the poxvirus has no coding sequence of the K7R gene. In a further embodiment the gene encoding the K7R gene is disrupted, mutated or truncated such that its gene product has reduced activity. In a further embodiment, one or more deletions or mutations in the promoter or other upstream sequences of the gene encoding the K7 polypeptide cause expression of the K7R gene to be compromised, leading to reduced or no levels of gene expression.

In one embodiment, the poxvirus further comprises within its genome, at least one non-poxvirus gene or a fragment of a non-poxvirus gene which gene or fragment encodes an antigen or a fragment thereof.

In a yet further aspect of the invention there is further provided a vaccine composition, wherein said immunogenic determinant is a recombinant poxvirus wherein the genome has been altered such that said poxvirus does not express a functional K7 protein from expression of the K7R gene.

In one embodiment the genome of the recombinant poxvirus further comprises a gene which encodes for an antigen or a fragment thereof.

Suitably, the recombinant poxvirus may be a vaccinia virus, a cowpox virus, a camelpox virus or an ectromelia virus or a derivative of any of those viruses.

In a further aspect of the invention there is provided the use of a poxvirus which lacks a functional gene encoding K7 for the manufacture of a vaccine for immunoprophylaxis of an infection caused by a poxvirus.

Suitably the poxvirus is an orthopoxvirus or a derivative thereof.

As used herein, the terms “inhibition” and “suppression” when used in relation to the modulation of the level of cytokine expression mean the partial or complete down-regulation of expression and/or activity of the cytokine and its expression levels.

The inventors have made the further surprising and unexpected finding that the K7 protein can induce cells of the immune system to express the cytokine IL-10

According to a further aspect of the present invention there is provided a composition for the prevention and/or treatment of a T cell mediated inflammatory immune response, said composition comprising a peptide comprising the amino acid sequence of SEQ ID NO:3 or an analogue, derivative, fragment, variant or peptidomimetic thereof

A yet further aspect of the present invention provides a pharmaceutical composition for the prevention and/or treatment of a T cell mediated inflammatory condition, wherein the composition comprises a peptide comprising the amino acid sequence of SEQ ID NO:3 or an analogue, derivative, fragment, variant or peptidomimetic thereof.

According to a yet further aspect of the present invention there is provided a method for modulating a T cell mediated immune response in a subject, the method comprising the step of;
administering an effective amount of an agent comprising a peptide comprising the amino acid sequence of SEQ ID NO:3 or an analogue, derivative, fragment, variant or peptidomimetic thereof.

In one embodiment of this aspect of the invention the T cell mediated immune response is suppressed. This suppression may result from a step of contacting an immune cell with an agent comprising a peptide comprising the amino acid sequence of SEQ ID NO:3 or an analogue, derivative, fragment, variant or peptidomimetic thereof, in accordance with the method of this aspect of the invention.

A further embodiment of the invention provides for the effective amount of the agent comprising a peptide comprising the amino acid sequence of SEQ ID NO:3 or an analogue, derivative, fragment, variant or peptidomimetic thereof to couple, bind or otherwise associate with a cell surface activation molecule of at least one type of immune cell, this resulting in the suppression, inhibition or down-regulation of one or more functional activities of that cell.

In one embodiment of the invention the immune cell whose function is modulated is at least one cell of the innate immune system.

In a further embodiment the cell is a cell type with antigen processing and presenting function, such as an antigen presenting cell (APC), for example a dendritic cell, or a macrophage or a B cell.

Where the APC is a dendritic cell it may be an immature dendritic cell, a semi-mature dendritic cell or it may be a mature dendritic cell.

In a further embodiment the cell of the innate immune system is a cell which does not function as an antigen presenting cell, for example a mast cell. Mast cells secrete cytokines such IL-4 and are accordingly known to have a role in facilitating the immune response, however they do not have an associated antigen processing function.

In one embodiment, the subject is a mammal. In a further embodiment, the mammal is a human.

A yet further aspect of the present invention provides a method of inducing the production of the cytokine IL-10 by the cells of the immune system, the method comprising the step of:
administering an effective amount of an agent comprising a peptide comprising the amino acid sequence of SEQ ID NO:3 or an analogue, derivative, fragment, variant or peptidomimetic thereof.

### Combinatorial Library

Combinatorial library technology (Schultz, J S (1996) Biotechnol. Prog. 12:729-743) provides an efficient way of testing a potentially vast number of different substances for ability to modulate activity of a polypeptide. Prior to or as well as being screened for modulation of activity, test substances may be screened for ability to interact with the polypeptide, e.g. in a yeast two-hybrid system (which requires that both the polypeptide and the test substance can be expressed in yeast from encoding nucleic acid). This may be used as a coarse screen prior to testing a substance for actual ability to modulate activity of the polypeptide.

The amount of test substance or compound which may be added to an assay of the invention will normally be determined by trial and error depending upon the type of compound used. Typically, from about 0.01 to 100 nM concentrations of putative inhibitor compound may be used, for example from 0.1 to 10 nM. Greater concentrations may be used when a peptide is the test substance.

### Identification of Inhibitors

Compounds which may be used may be natural or synthetic chemical compounds used in drug screening programmes. Extracts of plants which contain several characterised or uncharacterised components may also be used. A further class of putative inhibitor compounds can be derived from the K7 polypeptide and/or a ligand which binds the same. Peptide fragments of from 5 to 40 amino acids, for example from 6 to 10 amino acids from the region of the relevant polypeptide responsible for interaction, may be tested for their ability to disrupt such interaction.

Other candidate inhibitor compounds may be based on modelling the 3-dimensional structure of a polypeptide or peptide fragment and using rational drug design to provide potential inhibitor compounds with particular molecular shape, size and charge characteristics.

Following identification of a substance which modulates or affects K7 or DDX3 polypeptide activity, the substance may be investigated further. Furthermore, it may be manufactured and/or used in preparation, i.e. manufacture or formulation, of a composition such as a medicament, pharmaceutical composition or drug. These may be administered to individuals.

### Pharmaceutical Compositions

The present invention extends in various aspects not only to a substance identified as a modulator of polypeptide activity, in accordance with what is disclosed herein, but also to a pharmaceutical composition, medicament, drug or other composition comprising such a substance, a method comprising administration of such a composition to a patient, e.g. for treatment (which may include preventative treatment) of infection by a virus which expressed the K7 protein, use of such a substance in manufacture of a composition for administration, e.g. for treatment of a virus which expressed the K7 protein, and a method of making a pharmaceutical composition comprising admixing such a substance with a pharmaceutically acceptable excipient, vehicle or carrier, and optionally other ingredients.

### Mimetics

A substance identified as a modulator of K7 or DDX3 polypeptide function may be peptide or non-peptide in nature. Non-peptide “small molecules” are often preferred for many in vivo pharmaceutical uses. Accordingly, a mimetic or mimic of the substance (particularly is a peptide) may be designed for pharmaceutical uses. The designing of mimetics to a known pharmaceutically active compound is a known approach to the development of pharmaceuticals based on a “lead” compound. This might be desirable where the active compound is difficult or expensive to synthesise of where it is unsuitable for a particular method of administration, e.g. peptides are not well suited as active agents for oral compositions as they tend to quickly degraded by proteases in the alimentary canal. Mimetic design, synthesis and testing may be used to avoid randomly screening large numbers of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled according to its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. The mimetic or mimetics found by this approach can then be screened to see whether they have the target property, or to what extent they exhibit it. Further optimisation or modification can then be carried out to arrive at one or more final mimetics for in vivo or clinical testing.

Mimetics of substances having ability to modulate the K7 polypeptide, K7R promoter activity or the DDX3 peptide identified using a screening method as disclosed herein are included within the scope of the present invention. A polypeptide, peptide or substance which can modulate the activity of a polypeptide according to the present invention may be provided in a kit, e.g. sealed in a suitable container which protects its contents from the external environment. Such a kit may include instructions for use.

### Peptidomimetics

Whilst numerous strategies to improve the pharmaceutical properties of peptides found to exert biological effects are known in the art including, for example, amide bond replacements, incorporation of non-peptide moieties, peptide small molecule conjugates or backbone cyclisation, the optimisation of pharmacological properties for particular peptides still presents those involved in the optimisation of such pharmaceutical agents with considerable challenges.

Peptides of and for use in the present invention may be modified such that they comprise amide bond replacement, incorporation of non peptide moieties or backbone cyclisation.

Suitably if cysteine is present the thiol of this residue is capped to prevent damage of the free sulphate group.

Suitably a peptide of and for use in the present invention may be modified from the natural sequence to protect the peptides from protease attack.

Suitably a peptide of and for use in the present invention may be further modified using at least one of C and/or N-terminal capping, and/or cysteine residue capping. Suitably a peptide of and for use in the present invention may be capped at the N terminal residue with an acetyl group.

Suitably a peptide of and for use in the present invention may be capped at the C terminal with an amide group. Suitably the thiol groups of cysteines are capped with acetamido methyl groups.

### Sequence Homology

Particularly preferred nucleotide sequences of the invention are the sequences set forth in SEQ ID NO:2 and 4. The sequences of the amino acids encoded by the DNA of SEQ ID NO:2 and 4 are shown in SEQ ID NO:1 and 3 respectively.

Due to the known degeneracy of the genetic code, wherein more than one codon can encode the same amino acid, a DNA sequence can vary from that shown in SEQ ID NO:2 or 4 and still encode a polypeptide having the amino acid sequence of SEQ ID NO:1 or 3 respectively. Such variant DNA sequences can result from silent mutations (e.g., occurring during PCR amplification), or can be the product of deliberate mutagenesis of a native sequence.

The invention thus provides isolated DNA sequences encoding polypeptides of the invention, selected from: (a) DNA comprising the nucleotide sequence of SEQ ID NO:2 or 4 (b) DNA encoding the polypeptide of SEQ ID NO:1 or 3 (c) DNA capable of hybridization to a DNA of (a) or (b) under conditions of moderate stringency and which encodes polypeptides of the invention; (d) DNA capable of hybridization to a DNA of (a) or (b) under conditions of high stringency and which encodes polypeptides of the invention, and (e) DNA which is degenerate as a result of the genetic code to a DNA defined in (a), (b), (c), or (d) and which encodes polypeptides of the invention. Of course, polypeptides encoded by such DNA sequences are encompassed by the invention.

“Stringency” of hybridization reactions is readily determinable by one of ordinary skill in the art, and generally is an empirical calculation dependent upon probe length, washing temperature and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

As herein defined, “Stringent conditions”, “highly stringency conditions”, or “conditions of high stringency” may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5*SSC (0.75 M NaCl, 0.075 M sodium citrate), 50 mM sodium phosphate (pH 6.8), 0.1% sodium pyrophosphate, 5* Denhardt's solution, sonicated salmon sperm DNA (50 [mu]g/ml), 0.1% SDS, and 10% dextran sulfate at 42° C., with washes at 42° C. in 0.2*SSC (sodium chloride/sodium citrate) and 50% formamide at 55° C., followed by a high-stringency wash consisting of 0.1*SSC containing EDTA at 55° C.

“Moderately stringent conditions” or “conditions of moderate stringency” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5*SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1*SSC at about 37-50° C. The skilled artisan will recognize how to adjust the temperature, ionic strength, etc. as necessary to accommodate factors such as probe length and the like.

The invention thus provides equivalent isolated DNA sequences encoding biologically active forms of the K7 or DDX3 polypeptides selected from: (a) DNA derived from the coding region of K7 or DDX3; (b) DNA of SEQ ID NO:2 or 4, (c) DNA capable of hybridization to a DNA of (a) or (b) under conditions of moderate stringency and which encodes biologically K7 or DDX3 polypeptides; and (d) DNA that is degenerate as a result of the genetic code to a DNA defined in (a), (b) or (c), and which encodes biologically K7 or DDX3 polypeptides, such as those defined in SEQ ID NO:1 and 3 respectively.

As used herein, conditions of moderate stringency can be readily determined by those having ordinary skill in the art based on, for example, the length of the DNA. The basic conditions are set forth by Sambrook et al. Molecular Cloning: A Laboratory Manual, 2 ed. Vol. 1, pp. 1.101-104, Cold Spring Harbor Laboratory Press, (1989). Conditions of high stringency can also be readily determined by the skilled artisan based on, for example, the length of the DNA.

Also included as an embodiment of the invention is DNA encoding polypeptide fragments and polypeptides comprising inactivated N-glycosylation site(s), inactivated protease processing site(s), or conservative amino acid substitution(s).

In another embodiment, the nucleic acid molecules of the invention also comprise nucleotide sequences that are at least 80% identical to a native sequence. Also contemplated are embodiments in which a nucleic acid molecule comprises a sequence that is at least 90%, 91%, 92%, 93%, 94%, 95%, 96%, 97%, 98%, 99% or 99.5% identical to a native sequence.

The percent identity may be determined by visual inspection and mathematical calculation. Alternatively, the percent identity of two nucleic acid sequences can be determined by comparing sequence information using a computer programme. Alignment for purposes of determining percent amino acid sequence identity can be achieved in various ways using publicly available computer software such as BLAST or ALIGN. Those skilled in the art can determine appropriate parameters for measuring alignment, including any algorithms needed to achieve maximal alignment over the full length of the sequences being compared. In one embodiment, the GAP computer program, version 6.0 described by Devereux et al. (Nucl. Acids Res. 12:387, 1984) and available from the University of Wisconsin Genetics Computer Group (UWGCG) is used. The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp. 353-358, 1979; (2) a penalty of 3.0 for each gap and an additional 0.10 penalty for each symbol in each gap; and (3) no penalty for end gaps. Other programs used by one skilled in the art of sequence comparison may also be used.

The invention also provides isolated nucleic acids useful in the production of polypeptides. Such polypeptides may be prepared by any of a number of conventional techniques. A DNA sequence encoding the K7 or DDX3 polypeptide, or desired fragment thereof, may be subcloned into an expression vector for production of the polypeptide or fragment. The DNA sequence advantageously is fused to a sequence encoding a suitable leader or signal peptide. Alternatively, the desired fragment may be chemically synthesized using known techniques. DNA fragments also may be produced by restriction endonuclease digestion of a full length cloned DNA sequence, and isolated by electrophoresis on agarose gels. If necessary, oligonucleotides that reconstruct the 5′ or 3′ terminus to a desired point may be ligated to a DNA fragment generated by restriction enzyme digestion. Such oligonucleotides may additionally contain a restriction endonuclease cleavage site upstream of the desired coding sequence, and position an initiation codon (ATG) at the N-terminus of the coding sequence.

The invention encompasses polypeptides and fragments thereof in various forms, including those that are naturally occurring or produced through various techniques such as procedures involving recombinant DNA technology. For example, DNAs encoding K7 or DDX3 polypeptides can be derived from SEQ ID NO:2 or 4 by in vitro mutagenesis, which includes site-directed mutagenesis, random mutagenesis, and in vitro nucleic acid synthesis. Such forms include, but are not limited to, derivatives, variants, and oligomers, as well as fusion proteins or fragments thereof.

The polypeptides of the invention include full length proteins encoded by the nucleic acid sequences of SEQ ID NO:2 and 4. In certain embodiments, polypeptides comprise the amino acid sequences of SEQ ID NO:1 and 3 respectively.

Also provided herein are polypeptide fragments of varying lengths. Naturally occurring variants as well as derived variants of the polypeptides and fragments are also provided herein.

A “K7 variant” or a “DDX3 variant” as referred to herein means a polypeptide substantially homologous to K7 or DDX3, but which has an amino acid sequence different from that of native K7 or DDX3 polypeptide because of one or more deletions, insertions, or substitutions. The variant has an amino acid sequence that preferably is at least 80% identical to a K7 or DDX3 polypeptide amino acid sequence, most preferably at least 90% identical. The percent identity may be determined, for example, by comparing sequence information using the GAP computer program, version 6.0 described by Devereux et al. (Nucl. Acids Res. 12:387, 1984) and available from the University of Wisconsin Genetics Computer Group (UWGCG).

Variants also include embodiments in which a polypeptide or fragment comprises an amino acid sequence that is at least 90% identical, at least 95% identical, at least 98% identical, at least 99% identical, or at least 99.9% identical to the preferred polypeptide or fragment thereof.

Variants include polypeptides that are substantially homologous to the native form, but which have an amino acid sequence different from that of the native form because of one or more deletions, insertions or substitutions. Particular embodiments include, but are not limited to, polypeptides that comprise from one to ten deletions, insertions or substitutions of amino acid residues, when compared to a native sequence.

A given amino acid may be replaced, for example, by a residue having similar physiochemical characteristics. Examples of such conservative substitutions include substitution of one aliphatic residue for another, such as Ile, Val, Leu, or Ala for one another; substitutions of one polar residue for another, such as between Lys and Arg, Glu and Asp, or Gln and Asn; or substitutions of one aromatic residue for another, such as Phe, Trp, or Tyr for one another. Other conservative substitutions, e.g., involving substitutions of entire regions having similar hydrophobicity characteristics, are well known.

Similarly, the DNAs of the invention include variants that differ from a native DNA sequence because of one or more deletions, insertions or substitutions, but that encode a biologically active polypeptide.

### Production of K7/DDX3 Polypeptides and Fragments Thereof

Expression, isolation and purification of the polypeptides and fragments of the invention may be accomplished by any suitable technique. The present invention also provides recombinant cloning and expression vectors containing DNA, as well as host cells containing the recombinant vectors. Expression vectors comprising DNA may be used to prepare the polypeptides or fragments of the invention encoded by the DNA. A method for producing polypeptides comprises culturing host cells transformed with a recombinant expression vector encoding the K7/DDX3 polypeptide, under conditions that promote expression of the polypeptide, then recovering the expressed polypeptides from the culture. The skilled man will recognise that the procedure for purifying the expressed polypeptides will vary according to such factors as the type of host cells employed, and whether the polypeptide is intracellular, membrane-bound or a soluble form that is secreted from the host cell.

Any suitable expression system may be employed. The vectors include a DNA encoding a polypeptide or fragment of the invention, operably linked to suitable transcriptional or translational regulatory nucleotide sequences, such as those derived from a mammalian, avian, microbial, viral, bacterial, or insect gene. Nucleotide sequences are operably linked when the regulatory sequence functionally relates to the DNA sequence. Thus, a promoter nucleotide sequence is operably linked to a DNA sequence if the promoter nucleotide sequence controls the transcription of the DNA sequence. An origin of replication that confers the ability to replicate in the desired (E. coli) host cells, and a selection gene by which transformants are identified, are generally incorporated into the expression vector.

In addition, a sequence encoding an appropriate signal peptide (native or heterologous) can be incorporated into expression vectors. A DNA sequence for a signal peptide (secretory leader) may be fused in frame to the nucleic acid sequence of the invention so that the DNA is initially transcribed, and the mRNA translated, into a fusion protein comprising the signal peptide. A signal peptide that is functional in the intended host cells promotes extracellular secretion of the polypeptide. The signal peptide is cleaved from the polypeptide during translation, but allows secretion of polypeptide from the cell.

Suitable host cells for expression of polypeptides include higher eukaryotic cells and yeast. Prokaryotic systems are also suitable. Mammalian cells, and in particular CHO cells are particularly preferred for use as host cells. Appropriate cloning and expression vectors for use with mammalian, prokaryotic, yeast, fungal and insect cellular hosts are described, for example, in Pouwels et al. Cloning Vectors: A Laboratory Manual, Elsevier, New York, (1986) (ISBN 0444904018).

### Purification

The invention also includes methods of isolating and purifying the polypeptides and fragments thereof. An isolated and purified K7/DDX3 polypeptide according to the invention can be produced by recombinant expression systems as described above or purified from naturally occurring cells. K7/DDX3 polypeptides can be substantially purified, as indicated by a single protein band upon analysis by SDS-polyacrylamide gel electrophoresis (SDS-PAGE).

### Analogues and Derivatives

The present invention extends to peptides which are derivates or homologues of K7/DDX3, such peptides may have a sequence which has at least about 30%, or 40%, or 50%, or 60%, or 70%, or 75%, or 80%, or 85%, or 90%, 95%, 98% or 99% homology to the sequence of K7. Thus, a peptide fragment of any one of the peptides of the invention may include 1, 2, 3, 4, 5 or greater than 5 amino acid alterations.

Moreover, or in addition, the peptide may consist of a truncated version of K7/DDX3 which has been truncated by 1, 2, 3, 4 or 5 amino acids.

As is well understood, homology at the amino acid level is generally in terms of amino acid similarity or identity. Similarity allows for ‘conservative variation’, such as substitution of one hydrophobic residue such as isoleucine, valine, leucine or methionine for another, or the substitution of one polar residue for another, such as lysine, glutamic acid for aspartic acid, or glutamine for asparagine.

Analogues of, and for use in, the invention as defined herein means a peptide modified by varying the amino acid sequence e.g. by manipulation of the nucleic acid encoding the protein or by altering the protein itself. Such derivatives of the amino acid sequence may involve insertion, addition, deletion and/or substitution of one or more amino acids.

### Antibodies

Novel compounds identified using the assays of the invention form a further independent aspect of the invention. Such compounds or modulators may be provided in pharmaceutical compositions.

A modulator, or compound which modulates as identified according to the assays of the present invention may be a peptide or non-peptide molecule such as a chemical entity or pharmaceutical substance. Where the modulator is a peptide it may be an antibody, an antibody fragment, or a similar binding fragment. Further, where the modulator is an antibody, preferably it is a monoclonal antibody.

A monoclonal antibody, antibody fragment or similar binding molecule with specificity for K7/DDX3, has utility in the inhibition of the function of K7/DDX3.

In the context of the present invention, an “antibody” should be understood to refer to an immunoglobulin or part thereof or any polypeptide comprising a binding domain which is, or is homologous to, an antibody binding domain.

An “antibody” is an immunoglobulin, whether natural or partly or wholly synthetically produced. The term also covers any polypeptide, protein or peptide having a binding domain that is, or is homologous to, an antibody binding domain. These can be derived from natural sources, or they may be partly or wholly synthetically produced.

The antibody may be an intact antibody or a fragment thereof. Fragments of a whole antibody can perform the function of antigen binding.

Examples of such binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site; (viii) bispecific single chain Fv dimers and (ix) multivalent or multispecific fragments constructed by gene fusion.

Antibodies can be modified in a number of ways and accordingly the term “antibody” should be construed as covering any binding member or substance having a binding domain with the required specificity.

The antibody of the invention may be a monoclonal antibody, or a fragment, derivative, functional equivalent or homologue thereof. The constant region of the antibody may be of any suitable immunoglobulin subtype.

The term “antibody” includes antibodies which have been “humanised” or produced using techniques such as CDR grafting. Such techniques are well known to the person skilled in the art.

### Production of Antibodies

Specific binding members of and for use in the present invention may be produced in any suitable way, either naturally or synthetically. Such methods may include, for example, traditional hybridoma techniques, recombinant DNA techniques, or phage display techniques using antibody libraries. Such production techniques would be known to the person skilled in the art, however, other antibody production techniques are described in Antibodies: A Laboratory Manual, eds. Harlow et al., Cold Spring Harbor Laboratory, 1988.

### Treatment/Therapy

The term ‘treatment’ is used herein to refer to any regimen that can benefit a human or non-human animal. The treatment may be in respect of an existing condition or may be prophylactic (preventative treatment). Treatment may include curative, alleviation or prophylactic effects.

More specifically, reference herein to “therapeutic” and “prophylactic” treatment is to be considered in its broadest context. The term “therapeutic” does not necessarily imply that a subject is treated until total recovery. Similarly, “prophylactic” does not necessarily mean that the subject will not eventually contract a disease condition.

Accordingly, therapeutic and prophylactic treatment includes amelioration of the symptoms of a particular condition or preventing or otherwise reducing the risk of developing a particular condition. The term “prophylactic” may be considered as reducing the severity or the onset of a particular condition. “Therapeutic” may also reduce the severity of an existing condition.

### Administration

K7/DDX3, or a variant, analogue or fragment thereof, for use in the present invention may be administered alone but will preferably be administered as a pharmaceutical composition, which will generally comprise a suitable pharmaceutical excipient, diluent or carrier selected depending on the intended route of administration.

K7/DDX3, or a variant, analogue or fragment thereof, for use in the present invention may be administered to a patient in need of treatment via any suitable route. The precise dose will depend upon a number of factors, including the precise nature of the form of K7 to be administered.

Route of administration may include; parenterally (including subcutaneous, intramuscular, intravenous, by means of, for example a drip patch), some further suitable routes of administration include (but are not limited to) oral, rectal, nasal, topical (including buccal and sublingual), infusion, vaginal, intradermal, intraperitoneally, intracranially, intrathecal and epidural administration or administration via oral or nasal inhalation, by means of, for example a nebuliser or inhaler, or by an implant.

In preferred embodiments, the composition is deliverable as an injectable composition, is administered orally, or is administered to the lungs as an aerosol via oral or nasal inhalation.

For administration via the oral or nasal inhalation routes, preferably the active ingredient will be in a suitable pharmaceutical formulation and may be delivered using a mechanical form including, but not restricted to an inhaler or nebuliser device.

For intravenous injection, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection, Lactated Ringer's injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

The composition may also be administered via microspheres, liposomes, other microparticulate delivery systems or sustained release formulations placed in certain tissues including blood. Suitable examples of sustained release carriers include semipermeable polymer matrices in the form of shared articles, e.g. suppositories or microcapsules. Implantable or microcapsular sustained release matrices include polylactides (U.S. Pat. No. 3,773,919; EP-A-0058481) copolymers of L-glutamic acid and gamma ethyl-L-glutamate (Sidman et al, Biopolymers 22(1): 547-556, 1985), poly (2-hydroxyethyl-methacrylate) or ethylene vinyl acetate (Langer et al, J. Biomed. Mater. Res. 15: 167-277, 1981, and Langer, Chem. Tech. 12:98-105, 1982).

Examples of the techniques and protocols mentioned above and other techniques and protocols which may be used in accordance with the invention can be found in Remington's Pharmaceutical Sciences, 18th edition, Gennaro, A. R., Lippincott Williams & Wilkins; 20th edition (Dec. 15, 2000) ISBN 0-912734-04-3 and Pharmaceutical Dosage Forms and Drug Delivery Systems; Ansel, H. C. et al. 7^{th} Edition ISBN 0-683305-72-7 the entire disclosures of which is herein incorporated by reference.

The invention further encompasses recombinant vectors that direct the expression of the nucleic acid molecules of SEQ ID NO:2 and 4 which encode for K7 and DDX3 respectively, or variants or fragment thereof, and further host cells stably or transiently transformed or transfected with these vectors. Also encompassed by the invention are vectors comprising nucleic acid molecules complementary to these sequences as well as nucleic acid molecules that hybridize to a denatured, double-stranded DNA comprising all or a portion of SEQ ID NO:2 or 4. Suitable viral vectors will be known to the person skilled in the art, however a review can be found at Thomas et al. Nature Reviews Genetics 4, 346-358 (2003).

Suitably, the viral vector may be an Adenoviral, Adeno-associated virus (AAV), retroviral vectors, Herpes Simplex Virus or poxvirus.

In one embodiment the vector may be a lentiviral vector. In a further embodiment the vector is Equine Infectious Anaemia Virus (EIAV). The lentiviral vector may be a human immunodeficiency viruses HIV-1 or HIV-2, simian immunodeficiency virus (SIV), non-primate viruses for example maedi-visna virus (MVV), feline immunodeficiency virus (FIV), equine infectious anaemia virus (EIAV), caprine arthritis encephalitis virus (CAEV) and bovine immunodeficiency virus (BIV)).

Examples of specific viral vectors which may be suitable for such delivery and targeting may be; (i) nonreplicative herpes simplex type 1 viruses (Poliani et al. Hum Gene Ther. 2001 May 20; 12(8):905-20); (ii) Semliki Forest virus, (Jerusalmi et al. Mol. Ther. 2003 December; 8(6):886-94) and (iii) adenovirus, (for example see Braciack et al. J. Immunol. 2003 Jan. 15; 170(2):765-74).

Any suitable expression system may be employed. The vectors include a DNA encoding a polypeptide or fragment of the invention, operably linked to suitable transcriptional or translational regulatory nucleotide sequences, such as those derived from a mammalian, microbial, viral, or insect gene.

Examples of regulatory sequences include transcriptional promoters, operators, or enhancers, an mRNA ribosomal binding site, and appropriate sequences that control transcription and translation initiation and termination. Nucleotide sequences are operably linked when the regulatory sequence functionally relates to the DNA sequence. Thus, a promoter nucleotide sequence is operably linked to a DNA sequence if the promoter nucleotide sequence controls the transcription of the DNA sequence. An origin of replication that confers the ability to replicate in the desired host cells, and a selection gene by which transformants are identified, are generally incorporated into the expression vector.

In further embodiments, naked plasmid DNA encoding for K7 or fragments, derivative, mimetics or analogues thereof may be directly administered.

### Pharmaceutical Compositions

As described above, the present invention extends to a pharmaceutical composition comprising K7, DDX3 or analogues thereof for the modulation of the immune response. Pharmaceutical compositions according to the present invention and for use in accordance with the present invention may comprise, in addition to active ingredient (i.e. the K7 peptide or DDX3 peptide), a pharmaceutically acceptable excipient, carrier, buffer stabiliser or other materials well known to those skilled in the art. Such materials should be non-toxic and should not interfere with the efficacy of the active ingredient. The precise nature of the carrier or other material will depend on the route of administration, which may be, for example, oral, intravenous, intranasal or via oral or nasal inhalation.

The formulation may be a liquid, for example, a physiologic salt solution containing non-phosphate buffer at pH 6.8-7.6, or a lyophilised or freeze dried powder.

### Dose

The K7/DDX3 peptide or an analogue, derivative, fragment, variant or peptide thereof according to the invention is preferably administered to an individual in a “therapeutically effective amount”, this being sufficient to show benefit to the individual.

The actual amount administered, and rate and time-course of administration, will depend on the nature and severity of what is being treated.

Prescription of treatment, e.g. decisions on dosage etc, is ultimately within the responsibility and at the discretion of general practitioners, physicians or other medical doctors, and typically takes account of the disorder to be treated, the condition of the individual patient, the site of delivery, the method of administration and other factors known to practitioners.

The optimal dose can be determined by physicians based on a number of parameters including, for example, age, sex, weight, severity of the condition being treated, the active ingredient being administered and the route of administration.

A “subject” in the context of the present invention includes and encompasses mammals such as humans, primates and livestock animals (e.g. sheep, pigs, cattle, horses, donkeys); laboratory test animals such as mice, rabbits, rats and guinea pigs; and companion animals such as dogs and cats. It is preferred for the purposes of the present invention that the mammal is a human.

The compounds of the present invention are preferably administered to a subject in a “therapeutically effective amount”, this being an amount sufficient to show benefit to the individual. In particular, the benefit may be the treatment or partial treatment or amelioration of at least one symptom associated with a pro-inflammatory immune response. Where the context demands, a “therapeutically effective amount” is an amount which induces, promotes, stimulates or enhances the development of an immune response.

As used herein, the term “prophylactically effective amount” relates to the amount of a composition or compound which is required to prevent the initial onset, progression or recurrence of an immune-mediated disease, or at least one symptom thereof in a subject following the administration of the compounds of the present invention.

As used herein, the term “treatment” and associated terms such as “treat” and “treating” means the reduction of the progression, severity and/or duration of a pro-inflammatory immune response, of a disease condition associated with such a condition, or of at least one symptom thereof. The term ‘treatment’ therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition.

As used herein, the term “subject” refers to an animal, preferably a mammal and in particular a human. In a particular embodiment, the subject is a mammal, in particular a human. The term “subject” is interchangeable with the term “patient” as used herein.

The terms “peptide”, “polypeptide” and “protein” are used herein interchangeably to describe a series of at least two amino acids covalently linked by peptide bonds or modified peptide bonds such as isosteres. No limitation is placed on the maximum number of amino acids which may comprise a peptide or protein. Furthermore, the term polypeptide extends to fragments, analogues and derivatives of a peptide, wherein said fragment, analogue or derivative retains the same biological functional activity as the peptide from which the fragment, derivative or analogue is derived.

Unless otherwise defined, all technical and scientific terms used herein have the meaning commonly understood by a person who is skilled in the art in the field of the present invention.

Throughout the specification, unless the context demands otherwise, the terms ‘comprise’ or ‘include’, or variations such as ‘comprises’ or ‘comprising’, ‘includes’ or ‘including’ will be understood to imply the inclusion of a stated integer or group of integers, but not the exclusion of any other integer or group of integers.

### DETAILED DESCRIPTION

The present invention will now be described with reference to the following examples which are provided for the purpose of illustration and are not intended to be construed as being limiting on the present invention, and further, with reference to the figures as described briefly below.

FIG. 1: K7 has sequence similarity to A52 and is very conserved within the poxvirus family. (A) Alignment of A52 (VACV_WR078) and K7 (VACV_WR039) proteins from VACV (WR strain). The proteins show 24% sequence identity and 49% sequence similarity. The C-terminus of the previously described truncation mutant of A52R (ΔA52 1-144) and the corresponding K7 truncation mutant (ΔK7 1-108) is marked with a vertical bar. (B) Alignment and phylogenetic tree of K7 orthologs. (C) The WR sequence of K7R was cloned into the mammalian expression vector PCMV-HA. Increasing amounts of pCMV-HA-K7R were transfected into HEK293T cells, cells were harvested 48 hours after transfection and SDS-PAGE and western blotting analysis was performed using a HA-specific antibody. (D) HEK293 cells were infected with the WR strain of VACV at MOI 10 and harvested at the indicated time points after transfection. SDS-PAGE and western blotting analysis was performed using K7-specific antiserum. (E) BS-C-1 cells were infected with different poxviruses, cells were harvested 16 hours after infection and western blot analysis was performed using K7-specific antiserum. Abbreviations: VACV: Vaccinia virus, WR: Western Reserve, AMVA: Acambis Modified Virus Ankara, TAN: Tian Tan, MVA: Modified Virus Ankara, COP: Copenhagen, RPXV: Rabbitpox virus, UTR: Utrecht strain, VARV: Variola, GAR: Garcia strain, BSH: Bangladesh strain, IND: India strain, CMLV: Camelpox CPXV: Cowpox, BR: Brighton Red strain, MPXV: monkeypox and ZRE: Zaire strain.

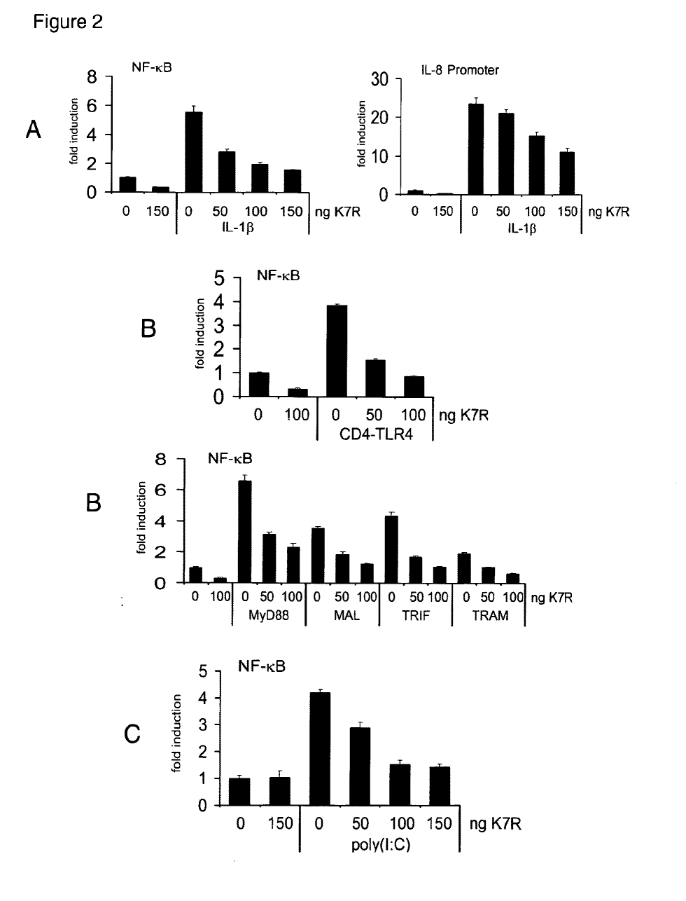

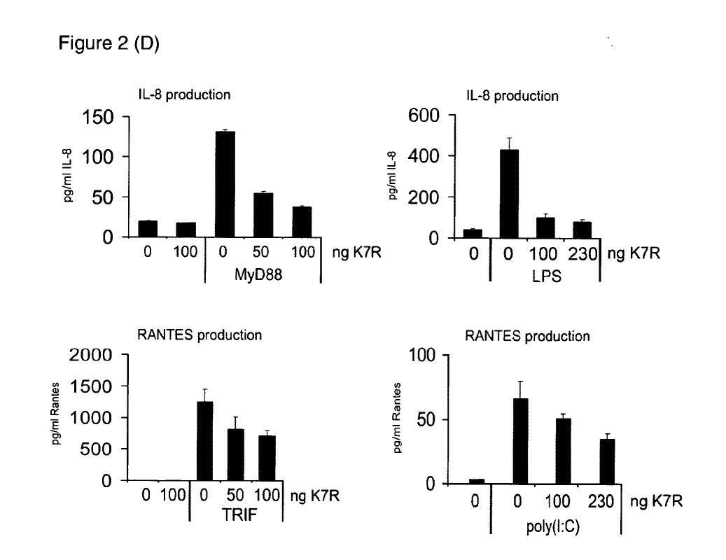

FIG. 2: K7 inhibits NF-κB activation induced by multiple TLR ligands. In all cases, HEK 293 cells were transfected with 0-230 ngs of pRK5-K7R, NF-κB or IL-8 promoter luciferase constructs and the phRL-TK Renilla control as described in Methods. The total amount of DNA was kept constant by addition of pRK5 empty vector. (A) As indicated, cells were stimulated with 20 ng/ml IL-1 6 hours prior to harvesting and measuring of luciferase reporter gene activity. (B) HEK 293 cells were transfected with 50 ng of CD4TLR4, MyD88, Mal, TRIF or TRAM expression constructs. 24 hours after transfection, cells were harvested and luciferase gene activity was measured. (C) To investigate TLR3 signalling to NF-κB, HEK293-TLR3 cells were transfected with the NF-κB luciferase constructs and the phRL-TK Renilla control as described in Methods. As indicated, cells were stimulated with 25 μg/ml poly(I:C) 8 hours prior to harvesting and measuring of luciferase gene activity. Data are expressed as the mean fold induction of luciferase activity relative to control levels. (D) HEK 293 cells were transfected with 50 ng of MyD88 or TRIF expression constructs. 48 hours after transfection supernatants were harvested and assayed for IL-8 or RANTES by ELISA. For agonist-induced cytokines, HEK-TLR4 or HEK-TLR3 cells were transfected with the indicated amounts of K7R 24 hours prior to stimulation with 1 μg/ml LPS or 25 μg/ml poly(I:C) respectively. 24 hours after stimulation supernatants were harvested and assayed for IL-8 or RANTES by ELISA.

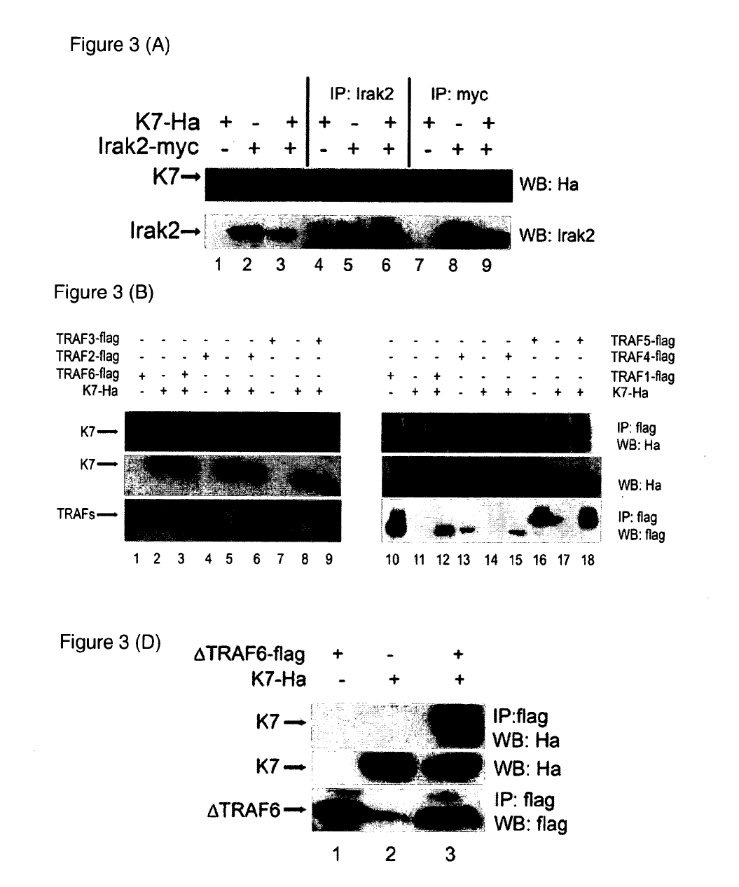

FIG. 3: K7 interacts with IRAK2 and TRAF6. (A) HEK 293T cells were transfected with K7R-HA and IRAK2-Myc as indicated. After 48 hours, lysates were subjected to immunoprecipitation (IP), SDS-PAGE and immunoblotting with the indicated antibodies. (B) HEK 293T cells were transfected with K7R-HA and Flag-TRAF1, 2, 3, 4, 5 or 6 as indicated. After 48 hours, lysates were subjected to IP, SDS-PAGE and immunoblotting with the indicated antibodies. (C) HEK 293T cells were transfected with K7R-HA and the Flag-tagged TRAF-domain of TRAF6 (ΔTRAF6) as indicated. After 48 hours, lysates were subject to IP, SDS-PAGE and immunoblotting with the indicated antibodies.

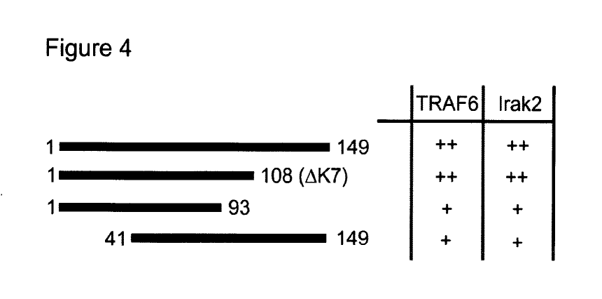

FIG. 4: Mapping of TRAF6 and IRAK2 binding site. Two different C-terminal and one N-terminal truncation mutant of K7R were constructed as indicated and the interaction of these mutants with TRAF6 and IRAK2 was investigated by immunoprecipitation experiments. The results of these experiments are summarized in a table with ++ indicating a strong interaction and + indicating an interaction.

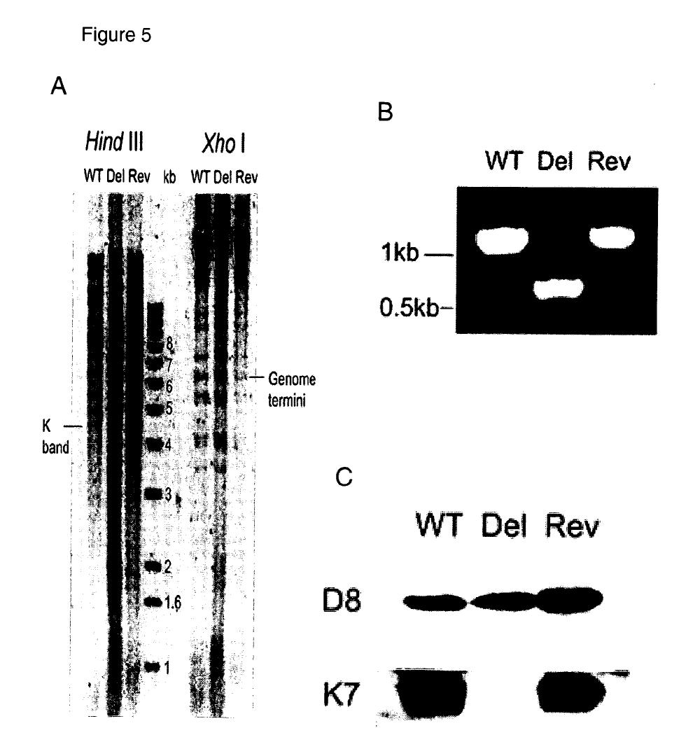

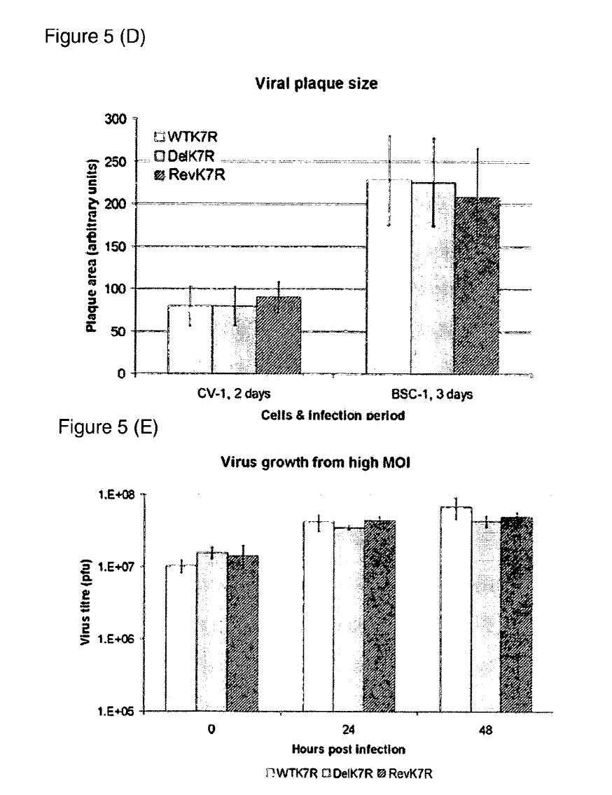

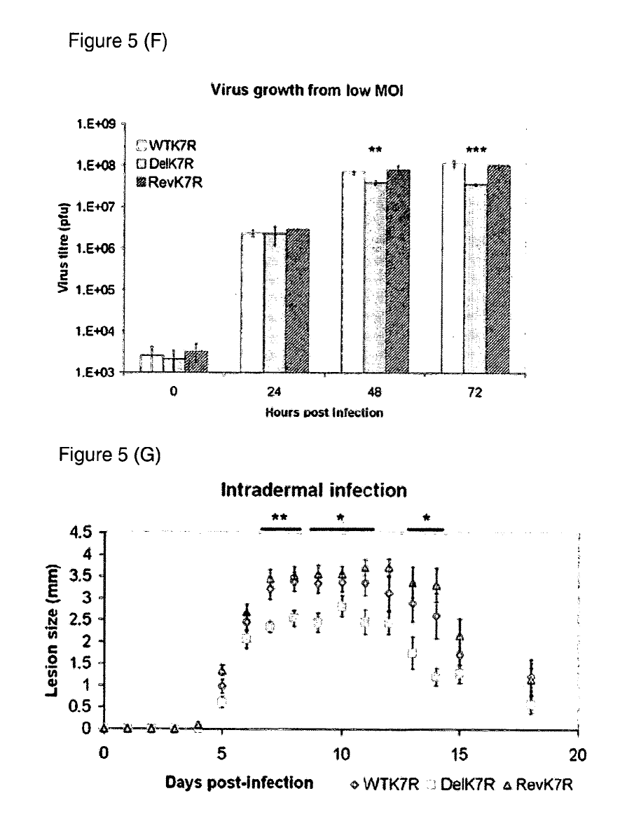

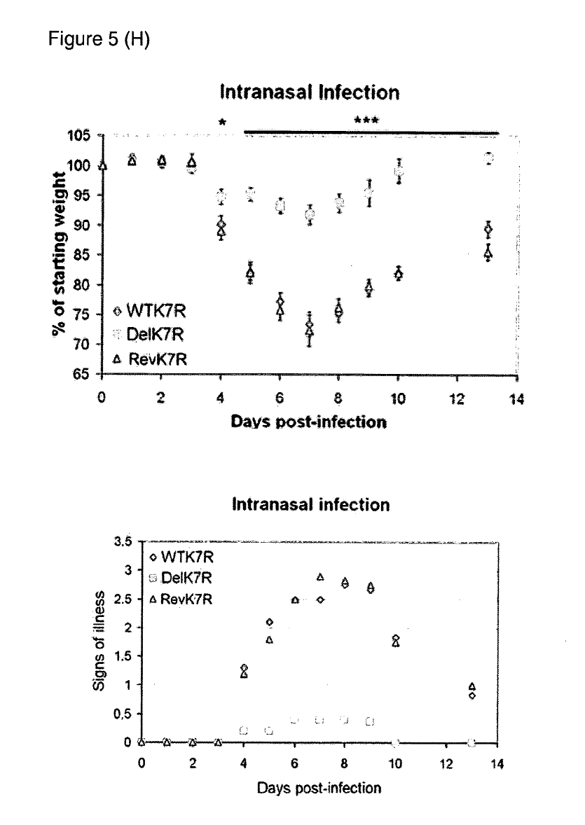

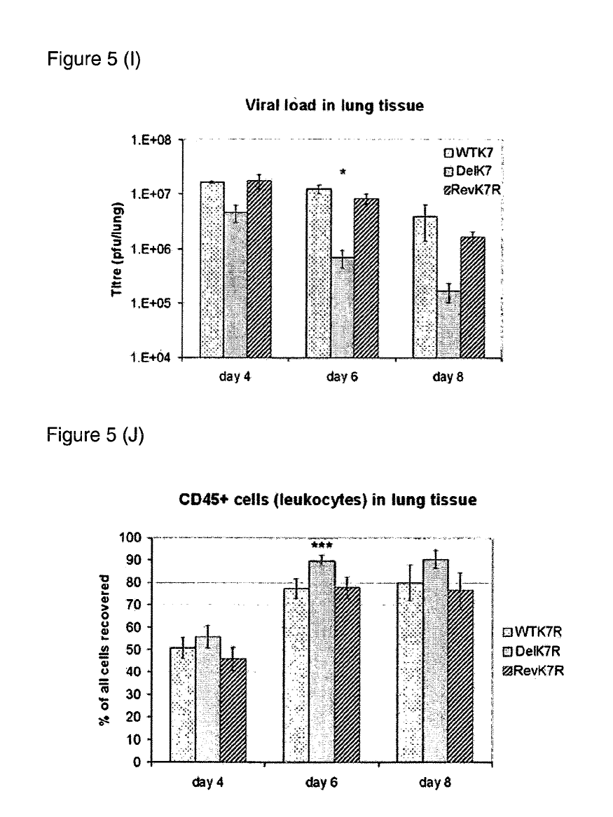

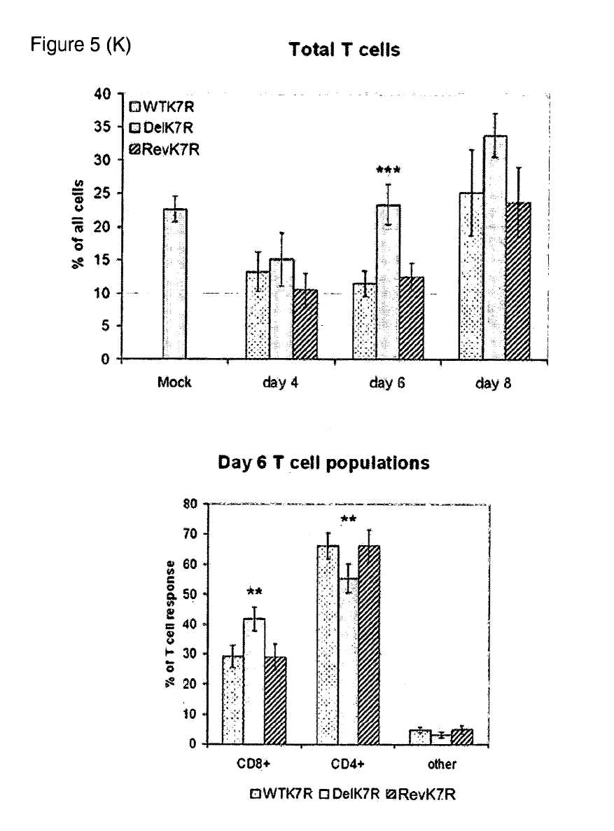

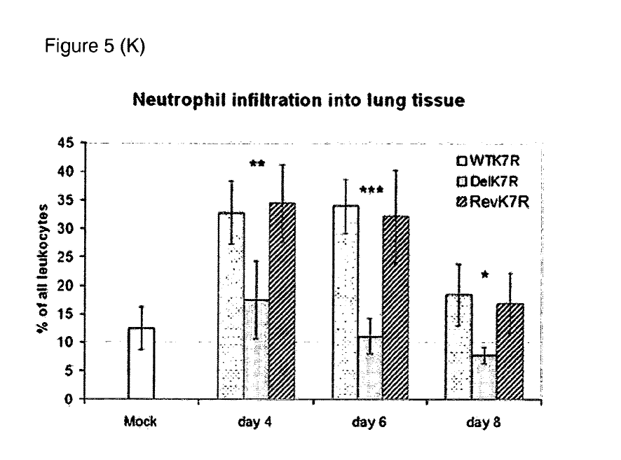

FIG. 5: A VACV lacking K7R shows a strong phenotype in vivo. All graphs: error bars show standard deviation. Statistical analyses were by one-factor ANOVA with Bonferroni post-tests for pairwise comparisons (* p>0.05, ** p>0.01, *** p>0.001). (A) Genomic DNA was purified from viruses vWTK7R (WT), vDelK7R (Del) and vRevK7R (Rev) and digested with Hind III or Xho I as indicated. Digestion products were separated on 0.5% agarose, ethidium bromide stained and visualised under UV light (negative image shown for clarity). (B) Primers 039U & 039D were used to amplify the locus containing K7R from the purified virus DNA as indicated. (C) HeLa cells were infected with the three viruses at MOI 1 and incubated for 24 hours before extracting protein with RIPA buffer. The extracts were Western blotted using antibodies against K7, and D8 (a virion structural protein) as a control for positive infection. (D) Cells were infected with 50-100 pfu of virus and incubated as shown, after which plaques were visualised microscopically and measured using ImagePro 4.0 analysis software. (E)-(F) BS-C-1 cells were infected with vDelK7R or control viruses and incubated as indicated before disrupting the cells by freeze-thawing and sonication and measuring the viral load by plaque titration. (E) Multiplicity of infection (MOI)=10; (F) MOI=0.015. (G) Groups of 5 C57Bl/6 mice were infected intradermally as indicated and lesion development monitored daily. (H) Groups of 5 Balb/c mice were infected intranasally and weight loss and signs of illness measured daily. (I)-(K) Groups of 5 mice were infected intranasally as before. On the days indicated lungs were removed and digested with Collagenase and DNase I, and passed through a 70 micron cell strainer. (I) Viral load in lung cell extracts was measured by plaque titration. (J) Following hypotonic lysis of red blood cells were washed, counted and stained for flow cytometry. (K) T cells were defined as small, non-granular cells which stained for CD3. Neutrophils were defined as larger, granular cells which were highly stained both for CD45 and Ly6G.

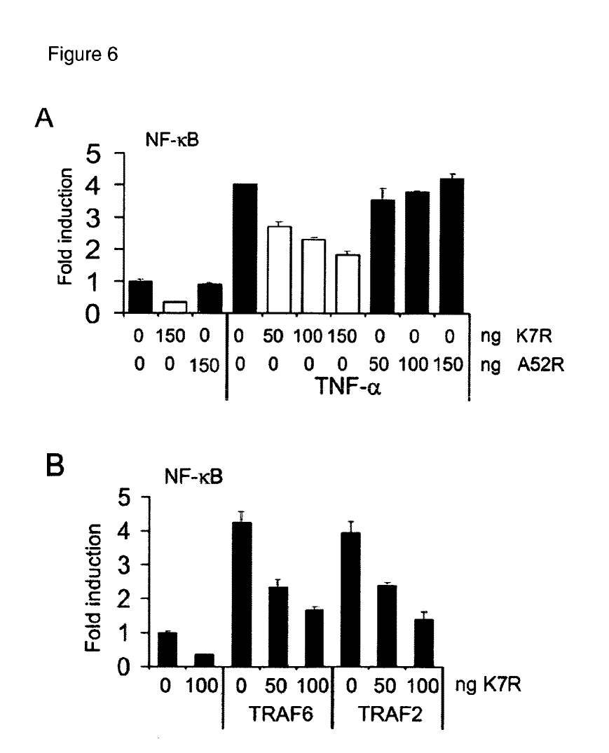

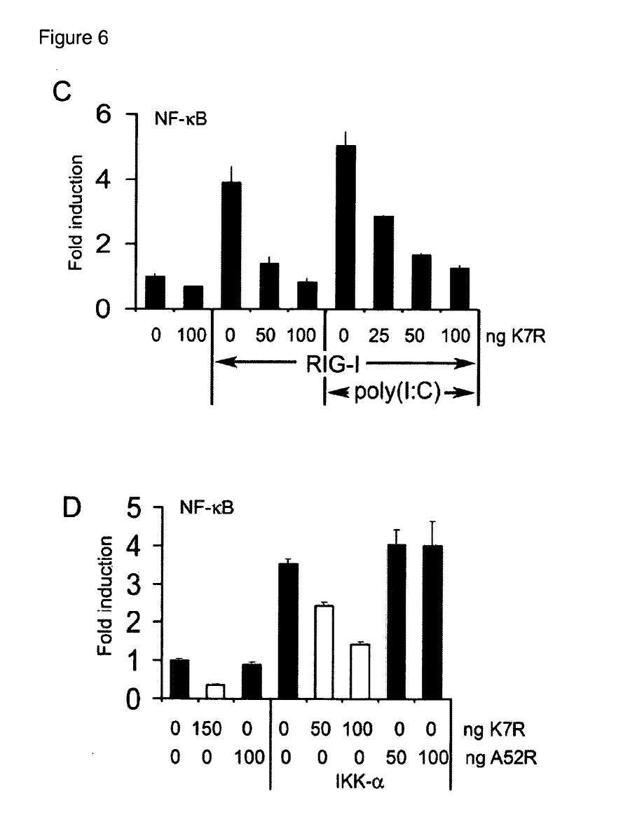

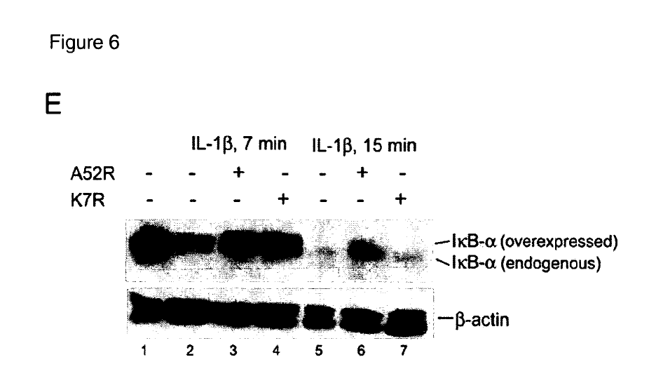

FIG. 6: K7 inhibits TLR-independent signalling to NF-κB. In A-D, HEK 293 cells were transfected with 0-150 ng of pRK5-K7R or pRK5-A52R as indicated, together with the NF-κB luciferase construct and the phRL-TK Renilla control. The total amount of DNA was kept constant by addition of empty vector pRK5. (A) As indicated, cells were stimulated with 20 ng/ml TNF-α 6 hours prior to harvesting and measuring of luciferase gene activity. (B) HEK 293 cells were transfected with 50 ng of TRAF6 or TRAF2 expression constructs as indicated. Cells were harvested and luciferase activity was measured 24 hours after transfection. (C) HEK293 cells were transfected with 50 ng of RIG-I expression construct. As indicated, the cells were transfected with 25 μg/ml poly(I:C) 15 hours prior to harvesting and measuring of luciferase gene activity was measured. (D) HEK293 cells were transfected with 50 ng of IKKα expression construct. Cells were harvested and luciferase activity was measured 24 hours after transfection. (E) HEK293-R1 cells were transfected with epitope-tagged IκB-α pRK5-K7R or pRK5-A52R. The total amount of DNA was kept constant by addition of empty vector pRK5. 24 hours after transfection, cells were stimulated with IL-1β for the indicated times, harvested and subjected to SDS-PAGE and western blotting with an antibody against IκB-α or β-actin as control for equal loading.

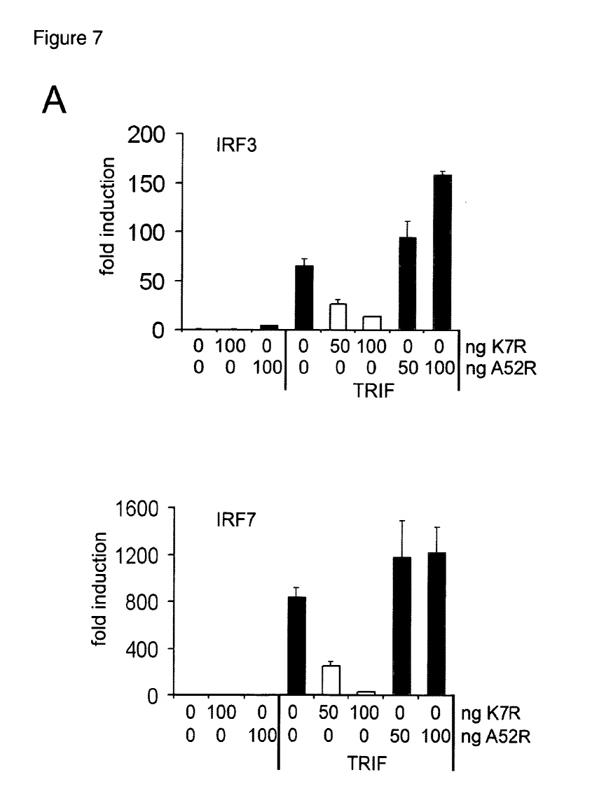

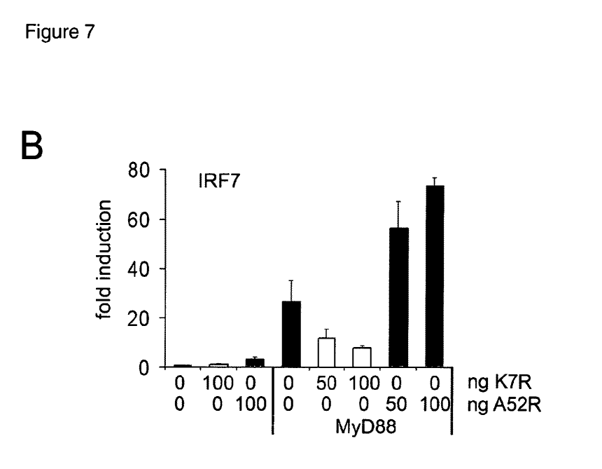

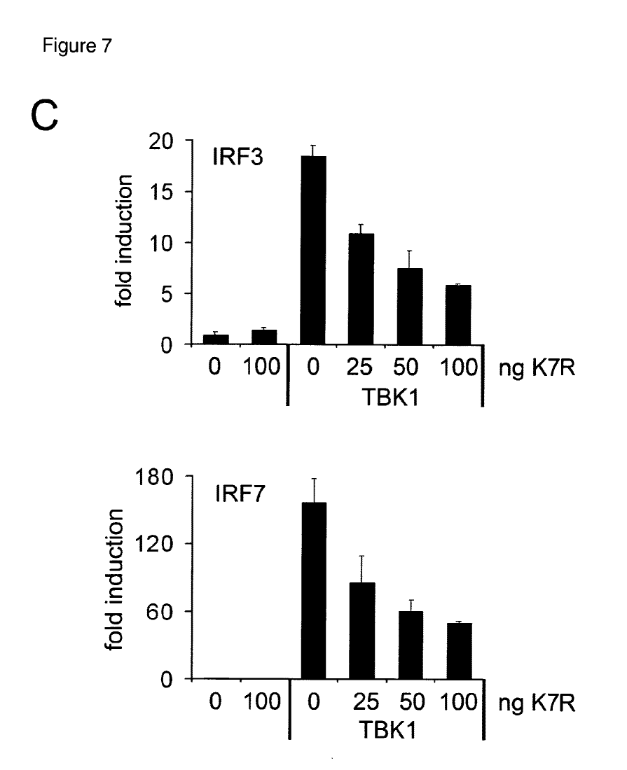

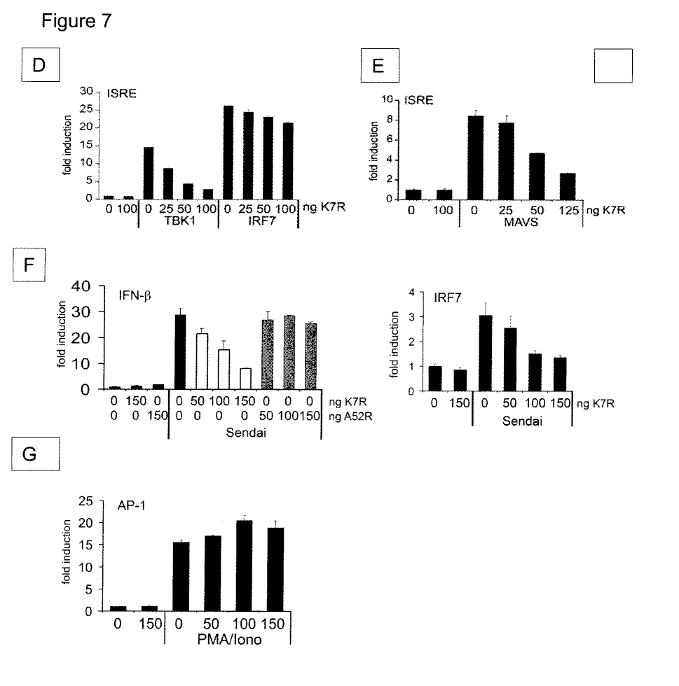

FIG. 7: K7 inhibits IRF activation. Data are mean fold induction of luciferase activity relative to control levels. In A-C, HEK 293T cells were transfected with 0-150 ng pRK5-K7R or 0-100 ng of pRK5-A52R as indicated. Cells were also transfected with 3 ng of the IRF3-GAL4 or the IRF7-GAL4 construct (as indicated) together with the pFR luciferase construct and the phRL-TK Renilla control as described in Methods. The total amount of DNA was kept constant by addition of empty vector pRK5. (A) HEK 293T cells were transfected with 50 ng of TRIF expression construct. Cells were harvested after 48 hours and luciferase reporter gene activity was measured. (B) HEK 293T cells were transfected with 50 ng MyD88 expression construct. Cells were harvested after 48 hours and luciferase reporter gene activity was measured. (C) HEK 293T cells were transfected with 50 ng TBK1 expression construct. Cells were harvested after 48 hours and luciferase reporter gene activity was measured. (D) Cells were transfected with the ISRE reporter construct and 50 ng of either TBK1 or IRF7 expression construct for 24 hours. (E) Cells were transfected with the ISRE reporter gene for 24 hours together with 50 ng MAVS expression construct. (F) HEK 293T cells were transfected with the IFN-β promoter reporter construct (left panel) or 3 ng IRF7-GAL4 construct and the pFR luciferase construct (right panel) and the phRL-TK Renilla control. 24 hours after transfection, Sendai virus was added to the supernatants. Cells were harvested 16 hours later and luciferase reporter gene activity was measured. (G) HEK 293 cells were transfected with 0-150 ng of pRK5-K7R, the AP-1 luciferase construct and the phRL-TK Renilla control. The total amount of DNA was kept constant by addition of empty vector pRK5. As indicated, cells were stimulated with PMA/lonomycin 15 hours prior to harvesting and measuring of luciferase gene activity.

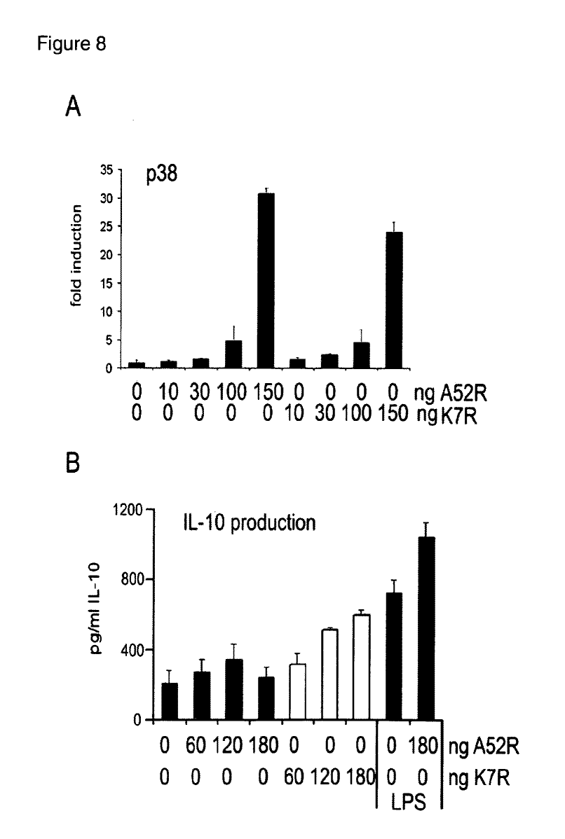

FIG. 8: K7 induces IL-10 production in RAW264.7 cells: (A) HEK 293-R1 cells were transfected with 0-150 ng of pRK5-K7R or pRK5-A52R, 0.25 ng of the CHOP-GAL4 expression construct, the pFR luciferase construct and the phRL-TK Renilla control as described in Methods. The total amount of DNA was kept constant by addition of empty vector pRK5. Cells were harvested after 24 hours and luciferase reporter gene activity was measured. (B) RAW264.7 were transfected with 0-180 ng of pRK5-K7R or pRK5-A52R. The total amount of DNA was kept constant by addition of empty vector pRK5. 24 hours after transfection cells were stimulated with 1 μg/ml LPS as indicated, supernatants were harvested 24 hours post-stimulation and assayed for IL-10 by ELISA.

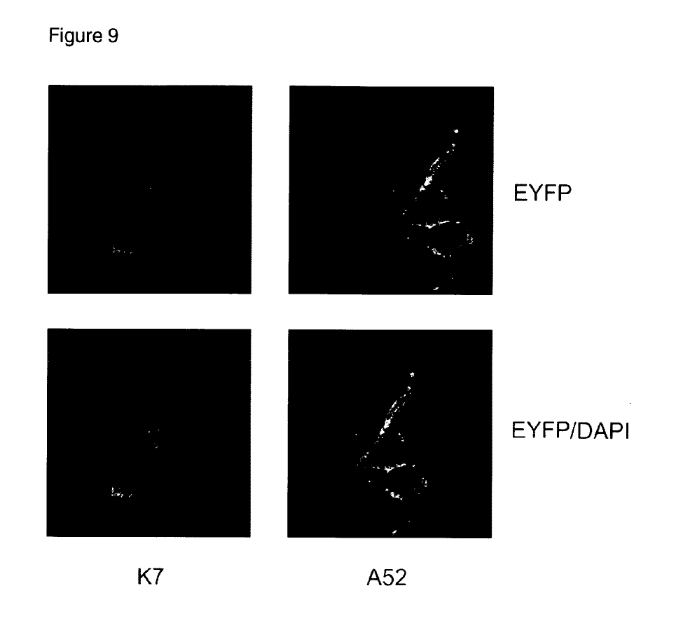

FIG. 9: K7 is present in both cytoplasm and nucleus. K7R-EYFP and A52R-EYFP constructs were transfected into HEK293 cells grown on glass coverslips and 48 hours after transfection, cells were fixed, permeabilized and stained with the DAPI nuclear stain before being analysed by confocal microscopy. Shown is a section of approximately 1 μm through the centre of the cell.

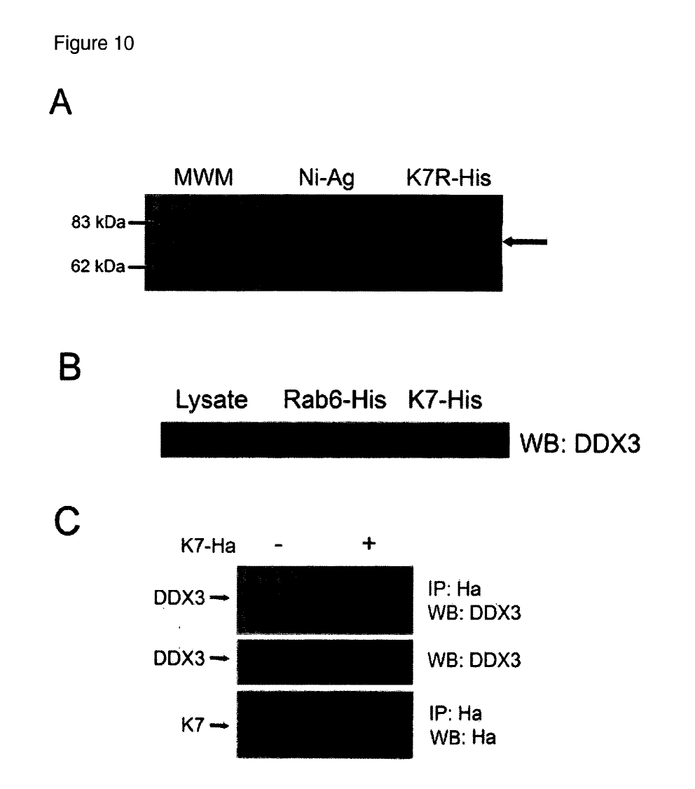

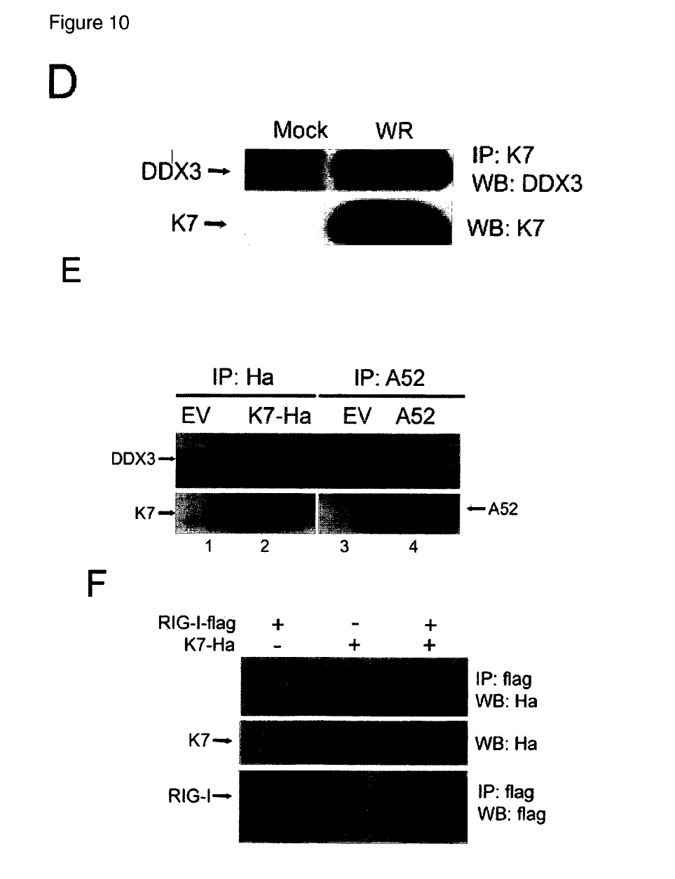

FIG. 10: K7 interacts with the cellular RNA helicase DDX3. (A) His-tagged K7 was expressed in BL21/DE3 E. coli and purified using Ni-Agarose. For pulldowns, HEK293 cell lysates were added to purified His-K7 coupled to Ni-Agarose and incubated for 2 hours at 4° C. The immune complexes were precipitated, subjected to SDS-PAGE and stained with Coomassie Blue. A band of approximately 70 kDa (marked with an arrow) that appeared specifically in the K7 pulldown lane was excised and prepared for MALDI-TOF analysis. (B) Pulldowns were performed as described above using His-tagged Rab6 as an unrelated control protein. After SDS-PAGE, western blot analysis was performed using an antiserum against DDX3. (C) HEK 293T cells were transfected with K7R-HA or empty vector. After 48 hours, lysates were subjected to IP with antibodies directed against the HA-tag followed by SDS-PAGE and immunoblotting with the indicated antibodies. (D) HEK293 cells were either infected with WR at an MOI of 5 or mock-infected. 16 hours post-infection, cell lysates were generated and subjected to IP with K7-antiserum, followed by SDS-PAGE and immunoblotting with the indicated antibodies. (E) HEK 293T cells were transfected with K7R-HA, A52R or EV. After 48 hours, lysates were subjected to IPs followed by SDS-PAGE and immunoblotting with the indicated antibodies. (F) HEK 293T cells were transfected with K7R-HA and Flag-RIG-I. After 48 hours, lysates were subjected to IPs followed by SDS-PAGE and immunoblotting with the indicated antibodies (E).

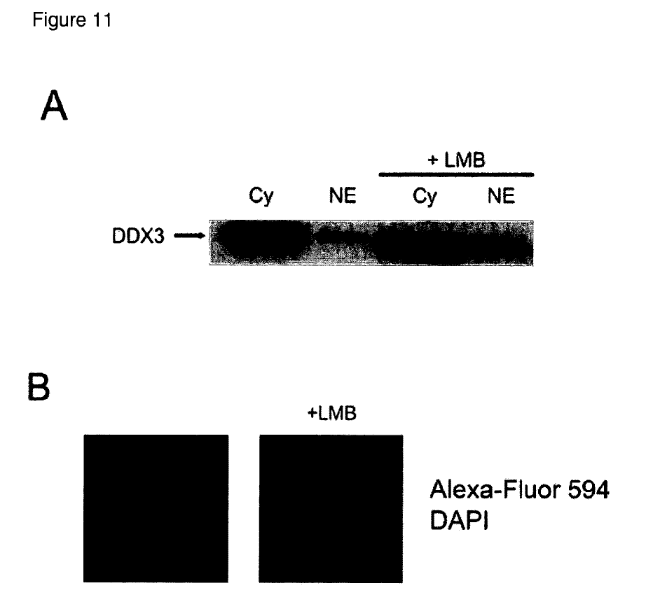

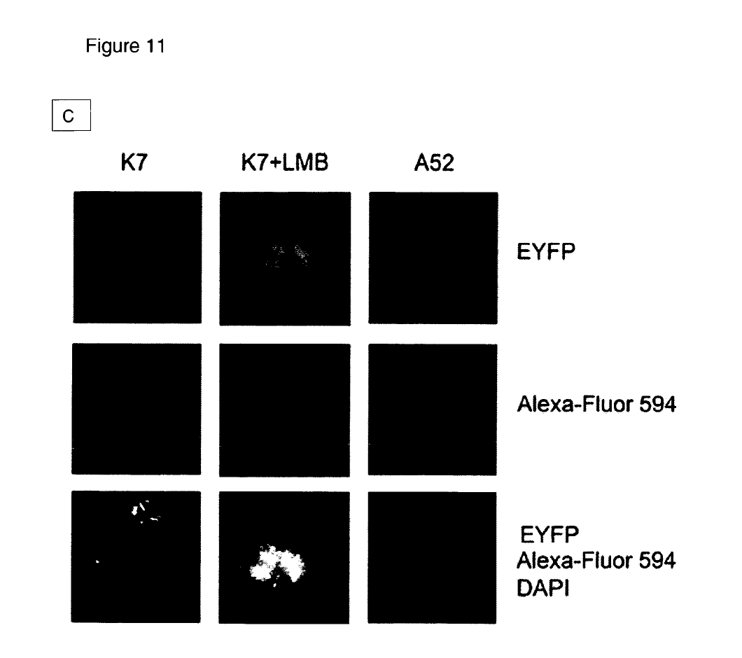

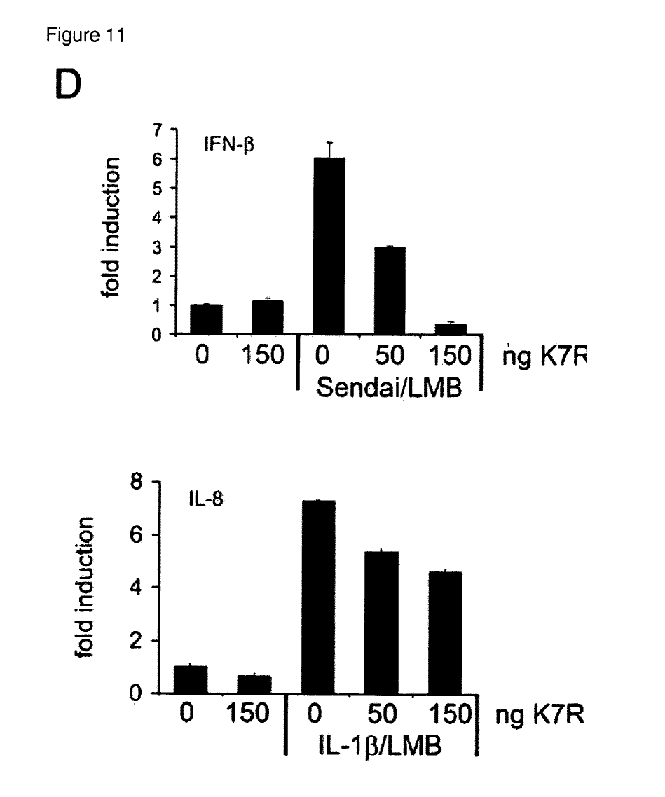

FIG. 11: K7 can interact with DDX3 in cytoplasm and nucleus. (A) Nuclear and cytoplasmic extracts were prepared from HEK293 cells that were either left untreated or treated with 25 nM Leptomycin B (LMB) for 4 hours at 37° C. These were then subjected to SDS-PAGE and western blotting with DDX3 antiserum. (B) HEK293 cells grown on glass coverslips were transfected with HA-DDX3. 4 hours before harvesting, cells were either treated with leptomycin B or left untreated. 48 hours after transfection, cells were fixed, permeabilised and stained with anti-HA-AlexaFluor594 and DAPI before being analysed by confocal microscopy. (C) HEK293 cells grown on glass coverslips were transfected with HA-DDX3 and K7R-EYFP or A52R-EYFP. 4 hours before harvesting, cells were either treated with leptomycin B or left untreated. 48 hours after transfection, cells were fixed, permeabilised and stained with anti-HA-AlexaFluor594 and DAPI before being analysed by confocal microscopy. (D) HEK 293 cells were transfected with 0-150 ng of pRK5-K7R as indicated, together with the IFN-β luciferase construct (left panel) or the IL-8 luciferase construct (right panel) and the phRL-TK Renilla control. The total amount of DNA was kept constant by addition of empty vector pRK5. LMB was added 2 hours prior to stimulation with Sendai virus (left panel) or IL-1 (right panel). Cells were harvested 12 hours post-stimulation and luciferase gene activity was measured.

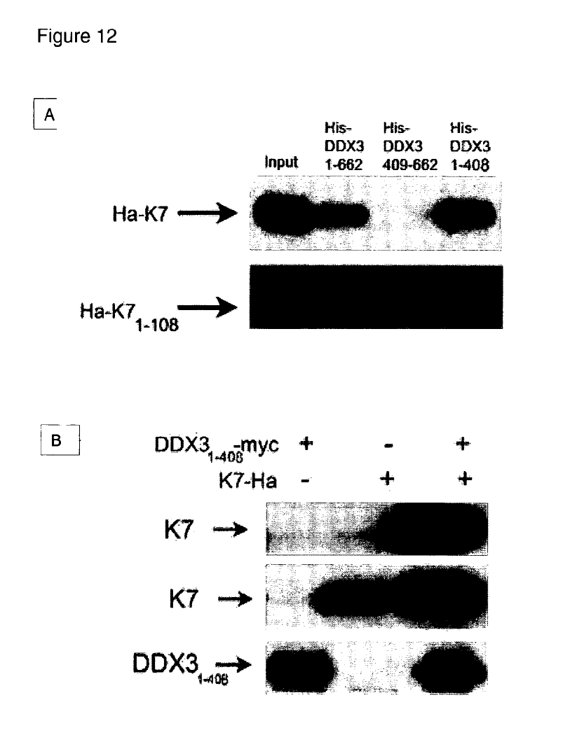

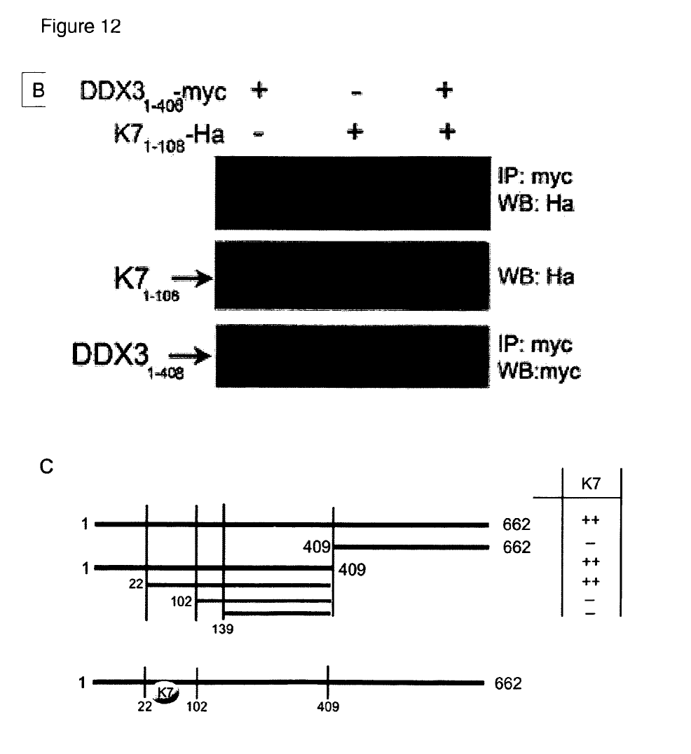

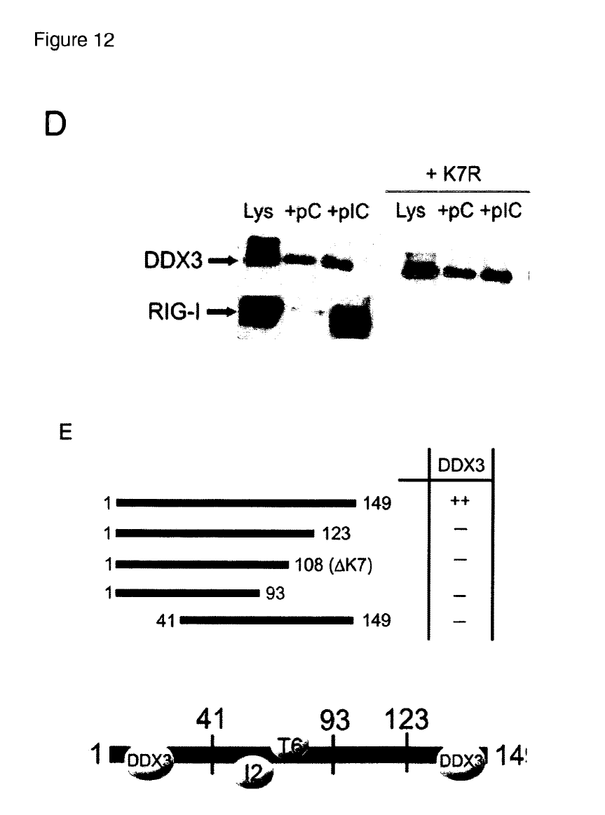

FIG. 12: Mapping of the interaction sites of K7 and DDX3. (A) Truncation mutants of DDX3 were expressed as His-fusion proteins in BL21 E. coli and purified using Ni-Agarose. For pulldowns, HEK293 were transfected with either HA-K7R or HA-ΔK7R (1-108), harvested and lysed after 48 hours as described for IPs. Cell lysates were then divided, added to the purified His-DDX3 coupled to Ni-Agarose and incubated for 2 hours at 4° C. The immune complexes were precipitated and subjected to SDS-PAGE and western blotting using the anti-HA antibody. (B) HEK 293T cells were transfected with K7R-HA (left panel) or ΔK7R-HA (right panel) and Myc-DDX3 1-408. After 48 hours, lysates were subjected to IPs followed by SDS-PAGE and immunoblotting with the indicated antibodies. (C) A series of IP experiments was carried out to test the interaction of the DDX3 mutants with K7. The results of these experiments are summarized and a schematic drawing shows the putative site of interaction of K7 on DDX3. (D) HEK293T cells were transfected with either DDX3-Myc or RIG-1-Flag and pRK5-K7R were indicated. 48 hours after transfection, cell lysates were generated in the presence of RNase inhibitors, divided and added to either poly(I:C) or poly(C) beads. These pulldowns were rocked for 2 hours at 4° C., precipitated and subjected to SDS-PAGE and western blotting with the indicated antibodies. (E) A series of IP experiments was carried out to test the interaction of the K7 truncation mutants with DDX3. The results of these experiments are summarized and a schematic drawing shows the putative site of interaction of DDX3 on K7 (as well as the previously determined sites for IRAK2 and TRAF6). For (C) and (E) ++ indicates a strong interaction, + indicates an interaction and − indicates no interaction.

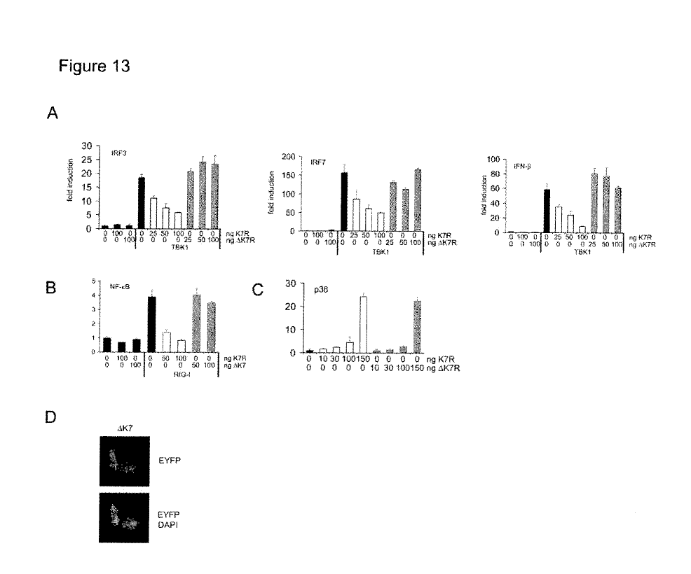

FIG. 13: ΔK7 fails to block NF-κB and IRF activation. In A-C, HEK 293T cells were transfected with 0-100 ng pRK5-K7R or pRK5-ΔK7R as indicated. The total amount of DNA was kept constant by addition of empty vector pRK5. (A) HEK293T cells were transfected with 3 ng of the IRF3-GAL4 (left panel) or the IRF7-GAL4 construct (middle panel) together with the pFR luciferase construct and the phRL-TK Renilla control or the IFN-β reporter construct (right panel) as well as 50 ng of the TBK1 expression construct where indicated. Cells were harvested after 48 hours and luciferase reporter gene activity was measured. (B) HEK 293 cells were transfected with the NF-κB reporter construct and the phRL-TK Renilla control along with 50 ng of RIG-I expression construct where indicated. Cells were harvested and luciferase activity was measured 48 hours after transfection. (C) HEK 293 cells were transfected with the IFN-β promoter reporter construct and the phRL-TK Renilla control along with 50 ng of TRIF expression construct. Cells were harvested and luciferase activity was measured 48 hours after transfection. (D) ΔK7R-EYFP(1-108) was transfected into HEK293 cells grown on glass coverslips and 48 hours after transfection, cells were fixed, permeabilized and stained with the DAPI nuclear stain before being analysed by confocal microscopy.

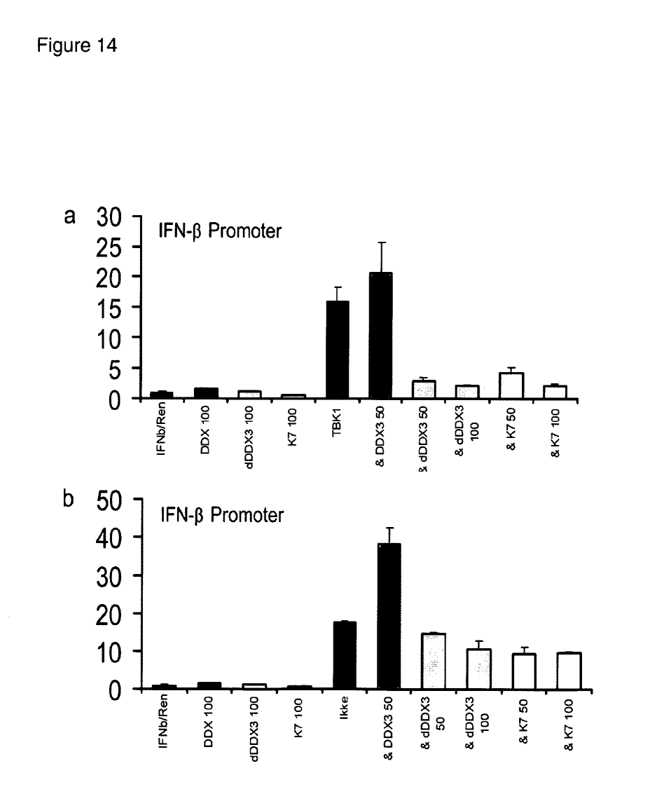

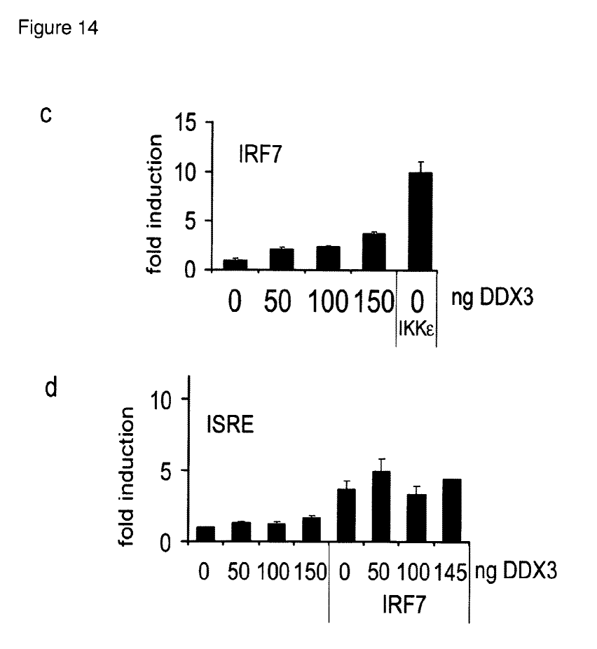

FIG. 14: DDX3 has a role in IRF activation. In (A)-(D), HEK293 cells were transfected with the indicated amount of pCMV-DDX3 (ng), together with the indicated luciferase reporter genes, and cells were harvested after 24 hours and luciferase reporter gene activity measured. Data are expressed as the mean fold induction of luciferase activity relative to control levels. (A) and (B) Cells were transfected with the IFN-β promoter reporter construct and 50 ng TBK1 (A) or IKK-ε (B) expression construct together with either K7 or dDDX3 (amino acids 408-662). (C) Cells were transfected with 3 ng of the IRF7-GAL4 construct, together with the pFR luciferase reporter, in order to measure the ability of DDX3 to activate IRF7. IKK-ε (50 ng) was used as a positive control. (D) Cells were transfected with the ISRE luciferase reporter, together with 5 ng IRF7 expression construct.

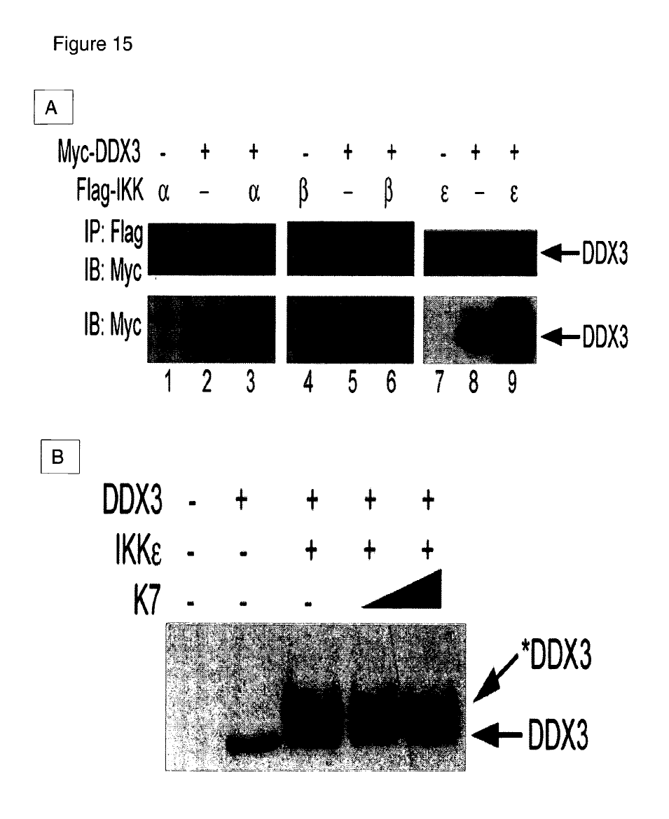

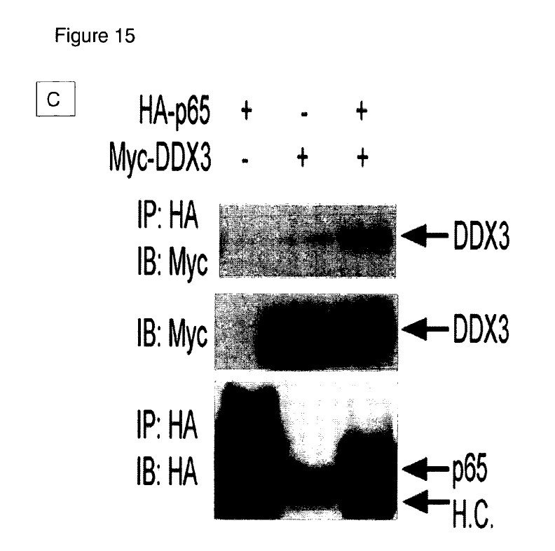

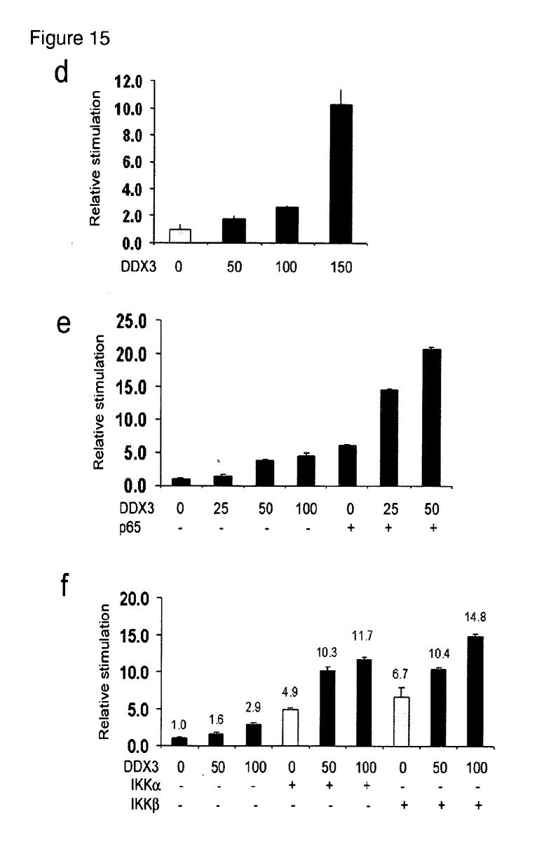

FIG. 15: (a) DDX3 can interact with IKK-α, IKK-β and IKK-ε. For co-immunoprecipitation experiments, 4 μg of flag-tagged IKK-α, IKK-β or IKK-ε and 4 μg of myc-tagged DDX3 were transfected into HEK293T cells seeded out in 100 mm dishes on the day before transfection. 48 hours after transfection, cells were harvested and lysed. The flag-tagged IKK was immunoprecipitated from the cell-lysates using anti-flag agarose. After thorough washing, samples were analysed by SDS-PAGE and western blotting and probed with anti-myc antibodies to detect DDX3. In the lower panels it can also be observed that co-expression of DDX3 with the IKKs leads to the appearance of a higher molecular weight band, indicating posttranslational modification (phosphorylation) of DDX3. (b). K7 reduces IKK-induced DDX3 phosphorylation. HEK293T cells were seeded out in 6-well plates on the day before transfection. They were then transfected with 0.25 μg DDX3 and 0.25 μg IKK-ε and increasing amounts of K7 (1 μg and 1.8 μg). The total amount of DNA was kept constant by addition of empty vector. Cells were harvested 24 hours after transfection, lysed and analysed by SDS-PAGE and western blotting. Blots were probed with anti-myc antibody to detect DDX3. Appearance of the upper, higher molecular weight band in the presence of an IKK indicates phosphorylation of DDX3, which is reduced in the presence of K7. (c) DDX3 interacts with the NF-κB subunit p65. For co-immunoprecipitation experiments, 4 μg of HA-tagged p65 and 4 μg of myc-tagged DDX3 were transfected into HEK293T cells seeded out in 100 mm dishes on the day before transfection. 48 hours after transfection, cells were harvested and lysed. p65 was immunoprecipitated from the cell-lysates using an antibody against the HA-tag. After thorough washing, samples were analysed by SDS-PAGE and western blotting and probed with anti-HA and anti-myc antibodies to detect p65 and DDX3 respectively. (d+e) Transactivation assays for p65 and p52. These assays are using a luciferase reporter gene under the control of the GAL4 transcription factor in conjunction with p65 or p52 fused to the DNA-binding domain of GAL4. (d) DDX3 can enhance transactivation activity of p65. One day before transfection HEK293s were seeded out in 96 well-plates. For the p65 transactivation assay, 0.5 ng of p65-GAL4 fusion vector was used in combination with 60 ng of the GAL4-dependent pFR-luciferase reporter construct. At the same time, different amounts of the DDX3 expression construct were also transfected (as indicated). Cells were harvested 24 hours after transfection and reporter gene activity was measured. (e) DDX3 can enhance transactivation activity of p52. For the p52 transactivation assay, 30 ng of p52-GAL4 fusion vector was used in combination with 100 ng of the pFR-luciferase reporter construct. To normalize for transfection efficiency, 20 ng of pGL3-Renilla were co-transfected in both cases. At the same time, different amounts of the DDX3 expression construct and 50 ng of HA-tagged p65 were co-transfected (where indicated). Cells were harvested 24 hours after transfection and reporter gene activity was measured. (f) DDX3 can activate an NF-κB dependent reporter gene. One day before transfection HEK293s were seeded out in 96 well-plates. Cells were transfected with the indicated amounts of myc-DDX3 or empty vector and 60 ng of the NF-κB luciferase reporter gene. To normalize for transfection efficiency, 20 ng of pGL3-Renilla were co-transfected. Cells were also co-transfected with 50 ng of IKK-α or IKK-β where indicated. Cells were harvested 24 hours after transfection and reporter gene activity was measured.

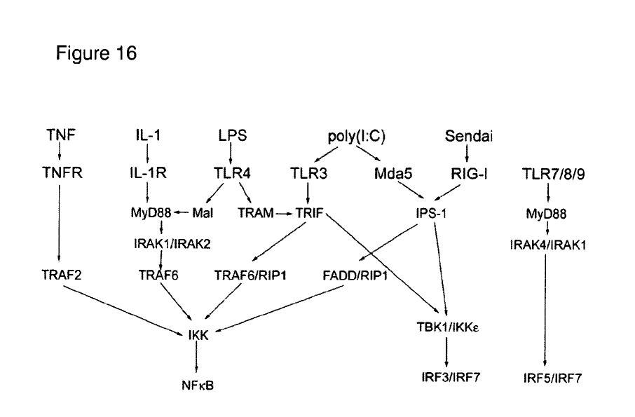

FIG. 16: Schematic of signalling pathways shown to be inhibited by K7.

### EXAMPLES

### Materials and Methods

### Expression Plasmids

Sources of expression plasmids were: Flag-TRAF6 and Flag-TRAF2 (Tularik Inc., San Francisco, Calif.), chimeric receptor CD4-TLR4 (R. Medzhitov, Yale University, CT), TLR3 (D. Golenbock, University of Massachusetts Medical School, Worcester, Mass.), Flag-TRIF (S. Akira, Osaka University, Osaka, Japan), Myc-MyD88 (L. O'Neill, Trinity College Dublin, Ireland), TBK1-Flag, RIG-I-Flag and IKKε-Flag (K. Fitzgerald, University of Massachusetts Medical School, Worcester, Mass.) and MAVS (Z. Chen, University of Texas, TX). K7R was amplified from genomic DNA of the WR strain of VACV using the following primers which introduce EcoRI and SalI restriction sites (restriction sites underlined, start and stop codons bold):

Sense (SEQ ID NO: 5):
5′CCGGAATTCAGATGGCGACTAAATTAGATTAT3′,
Antisense (SEQ ID NO: 6):
5′ACGCGTCGACTCAATTCAATTTTTTTTCTAG3′.

For the C-terminal truncation mutants, the following alternative antisense primers were used:

(SEQ ID NO: 7)

1-93: 5′ACGCGTCGACTCAATGGACTTTTGTAAAAT3′;

(SEQ ID NO: 8)

1-108 (ΔK7R): 5′ACGCGTCGACTCAGATTTTAGCACATATTCC3′;

(SEQ ID NO: 9)

1-123: 5′ACGCGTCGACTCATTGGAATCTAGATTCT3′.

For the N-terminal truncation mutant, the following alternative sense primer was used:

(SEQ ID NO: 10)

41-149: 5′CCGGAATTCAGATGATAGTGTTTAACAAAGATATT3′.

The PCR products were inserted into PCMV-HA to make HA-tagged K7R and HA-tagged K7R truncation mutants. HA-K7R and HA-ΔK7R were then subcloned into the pRK5 expression vector using an alternative sense primer, priming upstream of the HA-tag and introducing a BamHI site: 5′CGCGGATCCATGTACCCATACGATGTT3′ (SEQ ID NO:11). For expressing recombinant His-tagged K7R, K7R was cut from pCMV-HA and ligated into pH is Parallel-2. DDX3 was amplified from human PBMC cDNA with primers introducing EcoRI and SalI restriction sites:

(SEQ ID NO: 12)

sense: 5′GCCGAATTCGGATGAGTCATGTGGCA3′;

(SEQ ID NO: 13)

antisense: 5′ACGCGTCGACTCAGTTACCCCACCA3′.

DDX3 1-408 was cloned using the following alternative antisense primer:

(SEQ ID NO: 14)

5′ACGCGTCGACTGAAACTCTTCCTACAGCC3′.

DDX3 409-662 was cloned using the following alternative sense primer:

(SEQ ID NO: 15)

5′GCCGAATTCTTATGGGCTCTACCTCTGAAA3′.

PCR products were ligated into pCMV-Myc for making Myc-tagged DDX3 and DDX3 truncations. Full-length DDX3 was also sub-cloned into pCMV-HA for confocal staining work. For expressing recombinant His-tagged DDX3 and DDX3 truncations, DDX3 or the truncations were cut from pCMV-Myc and ligated into pH is Parallel-2. For generating EYFP-fusion proteins, K7R, AK7R and A52R were cloned in frame into pCDNA (Invitrogen) containing the EYFP ORF (provided by K. Kroeger, WAIMR, Perth, Australia) using the following antisense primers introducing XhoI restriction sites (and the EcoRI sense primers described above):

(SEQ ID NO: 16)

K7R: 5′ACGCCTCGAGTCCATTCAATTTTTTTTCTAG3′,

(SEQ ID NO: 17)

ΔK7R: 5′ACGCCTCGAGGATTTTAGCACATATTC3′;

(SEQ ID NO: 18)

A52R: 5′ACGCCTCGAGTGACATTTCCACATATA3′.

### Antibodies and Reagents

Anti-K7R polyclonal Ab was raised against purified full-length K7R expressed from pH is parallel 2-K7R in E. coli (Inbiolabs, Tallin, Estonia). Other antibodies used were anti-Flag M2 mAb, anti-Flag M2 conjugated agarose, anti-Myc mAb clone 9E10 (all from Sigma), anti-HA mAb (Covance, Cambridge Bioscience Limited, UK) and Anti-HA-AlexaFluor594 (Molecular Probes). Anti-DDX3 antiserum was kindly provided by the following sources: Yan-Hwa Wu Lee (National Yang-Ming University, Taipei, Taiwan), Arvind Patel (Glasgow, UK) and Kuan-The Jeang (Bethesda, Md., USA) and bought from Bethyl Laboratories, Texas, USA (clone BL1649). Human rIL-1α was a gift from the National Cancer Institute (Frederick, Wash., USA), while human rTNF-alpha was a gift from S. Foster (Zeneca Pharmaceuticals, Macclesfield, UK). TLR agonists used were poly(I:C) (Amersham Biosciences) and LPS (Sigma). Poly (I) and poly (C)-agarose were from Sigma. Sendai virus was provided by Søren R. Paludan (University of Aarhus, Denmark). Leptomycin B was purchased from Sigma and used at a final concentration of 25 nM.

### Reporter Gene Assays

HEK 293 cells (2×10⁴ cells per well) were seeded into 96-well plates and transfected 24 hours later with expression vectors and luciferase reporter genes using GeneJuice (Novagen). In all cases, 20 ng/well of phRL-TK reporter gene (Promega) was co-transfected to normalise data for transfection efficiency. The total amount of DNA per transfection was kept constant at 230 ng (HEK293) by addition of the corresponding empty vector (pRK5 or pCMV-HA) (Clontech). After 24 hours, reporter gene activity was measured (30). Data are expressed as the mean fold induction±SD relative to control levels, for a representative experiment from a minimum of three separate experiments, each performed in triplicate.

For NF-κB or IL-8 promoter reporter assays, 60 ng of a κB-luciferase reporter or an IL-8 promoter luciferase reporter gene respectively was used. For the MAP kinase reporter assay the Pathdetect System™ (Stratagene) was used, CHOP-GAL4 fusion vector (0.25 ng) was used in combination with 60 ng pFR-luciferase reporter to measure p38 activation. For the IRF assays, an IRF3-GAL4, IRF5-GAL4 or IRF7-GAL4 fusion vectors (3 ng) were used in combination with 60 ng of the pFR luciferase reporter.

### Immunoprecipitation and Immunoblotting

HEK293T cells were seeded into 10 cm dishes (1.5×10⁶ cells) 24 h prior to transfection with GeneJuice. For co-immunoprecipitations (co-IP), 4 g of each construct was transfected. Cells were harvested after 48 h in 850 l of lysis buffer (50 mM Tris/Cl pH 7.5, 100 mM NaCl, 1 mM EDTA, 10% glycerol, 0.5% NP-40 containing 0.01% aprotinin, 1 mM sodium orthovanadate and 1 mM PMSF). For assessment of interactions involving VACV-expressed K7R, cells were infected (multiplicity of infection, MOI=10) with VACV WR 24 hours post-transfection for 60 minutes at 37° C. The virus inoculum was aspirated, cell monolayers were overlaid with 2.5% FBS DMEM and harvested 4 hours post-infection in lysis buffer. For all IPs, the appropriate antibodies were precoupled to either protein A or protein G sepharose for 1 hour at 4° C., prior to incubation with the cell lysates for 2 hours at 4° C. The immune complexes were precipitated, washed and analysed by SDS-PAGE and immunoblotting. For analysis of the kinetics of K7R expression, confluent monolayers of HEK293 cells were infected (MOI=1=0) for 60 minutes at 37° C. After removal of inoculum, cell monolayers were overlaid with 2.5% FBS DMEM. Cell lysates were analysed by immunoblotting using anti-K7R antiserum.

### Determination of Cytokine Concentrations

HEK293 clonal cell lines expressing either TLR3 (HEK-TLR3) or TLR4 and MD-2 (HEK-TLR4) were used for determination of cytokine production. Cells (2×10⁴ cells per well) transfected with the K7R expression plasmid for 24 hours were stimulated with 1 μg/ml LPS or 25 μg/ml poly(I:C) 24 hours later. Supernatants were harvested 24 hours later and IL-8 and RANTES concentrations were determined by ELISA (R&D Biosystems). Experiments were performed four times in triplicate and data are expressed as the mean±SD from one representative experiment.

### Recombinant VACV Viruses

Plasmids for the generation of recombinant viruses were constructed using the following PCR primers:

(SEQ ID NO: 19)

039U: 5′-AACTTCTAGATTCACCATTACTTCTTCCATGTCC-3′

(SEQ ID NO: 20)

039D: 5′-TGATGAATTCGGGGTTGGGTGTAAGATTGG-3′

(SEQ ID NO: 21)

039N: 5′-CCCCTATATCAGACTATCTCACAAAAGACAGTAGC-3′

(SEQ ID NO: 22)

039C: 5′-GTGAGATAGTCTGATATAGGGGTCTTCATAACGC-3′

Wild type and modified VACV sequences were amplified by PCR from purified VACV WR genomic DNA and cloned into the EcoR I site of plasmid pSJH7. Primers 039U & 039D were used to amplify the K7R orf with 339 bp of upstream and 323 bp of downstream flanking sequence and produce plasmid pSJH7-K7R. The flanking sequences alone were amplified using primer pairs 039U & 039N, and 039C & 039D, respectively. The overlapping sequences at the 5′ ends of 039N and 039C (italicised) enabled the two flanking regions to be spliced together without the K7R orf in a second amplification using 039U & 039D, to produce pSJH7-DelK7R.

This latter plasmid was transfected into CV-1 cells infected with VACV WR and a recombinant virus lacking the K7R orf (vDelK7R) isolated by transient dominant selection as previously described and plaque purified, along with a wild-type virus (vWTK7R) derived from the same intermediate. Cells infected with vDelK7R were then transfected with pSJH7-K7R and selected to produce a revertant virus (vRevK7R) in which the K7R gene was reinserted at its natural locus. Virus infectivity and plaque morphology were assessed by plaque titration in duplicate on BS-C-1 cell monolayers which were infected for 1.5 hours with regular agitation and then incubated at 37° C. under a semi-solid overlay of 1.5% carboxymethylcellulose in 2.5% FBS DMEM for 48 hours prior to visualisation of plaques by staining with 0.1% (w/v) Crystal Violet in 15% (v/v) ethanol.

### Animal Infections

Intradermal inoculations of the ear pinnae of female, 6-8 week old C57Bl/6 mice were carried out as described previously. For intranasal infection female, 6-8 wk-old Balb/c mice were anaesthetized and inoculated with 10⁴ plaque-forming units of VACV in 20 μl of phosphate-buffered saline. A control group was mock-infected with phosphate-buffered saline. Each day the weights of the animals and signs of illness were measured as described previously. For lung cell analysis, mice were killed by lethal injection and lungs extracted immediately. Single cell suspensions were prepared in RPMI 1640 containing 10% (v/v) FBS by digesting lungs with 1 mg/ml collagenase A and 0.02% (w/v) DNase I (Sigma) for 30 minutes before passing through a 70-μm nylon mesh followed by hypotonic lysis of erythrocytes. Cell viability was assessed using trypan blue exclusion. The animal experiments were conducted under the appropriate licence and regulations stipulated by the Animals (Scientific Procedures) Act 1986, United Kingdom Government.

### Flow Cytometry

Fresh lung cells were washed twice in flow cytometry (FC) buffer (0.1% (w/v) BSA, 0.1% (w/v) NaN₃ in PBS) prior to blocking and staining (30 min each) in FC buffer containing 10% (v/v) normal rat serum and 1 μg/ml purified anti-mouse CD16/CD32 (BD) to block FcRγII/III receptors. Antibodies used were Allophycocyanin (APC)-conjugated rat anti-mouse CD45 (BD), Phycoerythrin (PE)-conjugated rat anti-mouse Ly6G (Caltag), Fluorescein isothiocyanate (FITC)-conjugated rat anti-mouse CD3 (BD), PE-Cy5 conjugated rat anti-mouse CD8a (Caltag), and APC-conjugated rat anti-mouse CD4 (Caltag). Stained cells were washed, then fixed using 1% paraformaldehyde in PBS before analysis on a BD FacsCalibur.

### His Pulldown Assays

Plasmids pHisparallel2-K7R, pHisparallel2-DDX3, pHisparallel2-DDX3 1-408 or pHisparallel2-DDX3 409-662, pET28-DDX3 139-408, pET28-DDX3 22-408, pET28-DDX3 102-408 were transformed into E. coli BL21 (DE3) and grown in Luria Bertani medium. Protein expression was induced with 0.7 mM IPTG. Cells were lysed by sonication in binding buffer (10 mM Tris/Cl pH 8, 300 mM NaCl, 20 mM imidazole, 0.5 mM PMSF, 10 mM DTT) and proteins were purified from the soluble fraction using Ni-Agaraose (Sigma). For His pulldown experiments, HEK 293T cells were transfected and harvested as described for co-IP. Cell lysate (800 μl) was added to purified His fusion protein coupled to Ni-Agarose and incubated for 2 hours at 4° C. The immune complexes were precipitated and subjected to SDS-PAGE. For the identification of K7 interacting proteins, specific bands were cut out of the Coomassie-stained gel and prepared for MALDI-TOF analysis. For all other pulldown experiments, gels were transferred to PVDF membranes and subjected to immunoblotting.

### MALDI-TOF

Bands were cut from Coomassie stained SDS-PAGE gels and washed in 50% acetonitrile and then destained in 0.1 M NH₄HCO₃. The protein in the gel was then alkylated by incubating with 10 mM DTT in 0.1M NH₄HCO₃ for 45 minutes at 65° C., followed by 30 minutes incubation at RT with 50 mM iodoactamide (Sigma). After extensive washing in 50% acetonitrile, 100% acetonitrile was added to dehydrate the gel pieces. After complete dehydration, 50 μl of a 10 μg/ml modified trypsin (Promega) solution was added and incubated overnight at 37° C. The next day, 50 μl acetonitrile was added and the samples were rocked for 30 min at RT. The supernatant was collected and the extraction was repeated once with 66% acetonitrile. The pooled supernatants were then taken to dryness in a vacuum centrifuge. These samples were analysed by MALDI-TOF and the resulting peptide peaklist was searched against the NCBI nr database using the Mascot search engine. DDX3 was identified with a Probability based Mowse score of 120.

### Confocal Imaging

HEK293 cells were grown on 22 mm coverslips in 6-well plates and transfected with 2.3 μg of total DNA (EYFP-fusion protein constructs with or without DDX3-HA) when 50% confluent. 48 hours after transfection, cells were washed, fixed with 4% paraformaldeyde (15 minutes on ice) and permeabilised with 0.5% Triton-X-100 (30 min on ice). They were then blocked for 1 hour at RT with 3% BSA, 0.05% Tween-20 in PBS. Staining with anti-HA-AlexaFluor594 was performed for 3 hours at RT in blocking solution. Nuclei were then stained with DAPI. Cells were washed and mounted onto glass slides with 2 mg/ml p-phenyldiamine in 50% glycerol/PBS. The slides were examined by phase contrast and confocal microscopy with an Olympus Fluoview FV1000 Imaging system using a 60× objective and 2-3 fold digital zoom. The excitation light for imaging was provided by the 457-514 nm lines of a multi-line Argon laser, the 543 nm line of a Green Helium-Neon laser and the 633 nm line of a Red Helium-Neon laser. Images were collected with Kalman integration and processed with the Olympus Fluoview software (Version 1.3c).

Pulldowns with poly(I:C)

Pulldowns were performed as previously described. In brief, a solution of poly (I) was prepared at 2 mg/ml in binding buffer (60 mM Tris/Cl pH 7, 150 mM NaCl). Poly (C)-agarose beads were rehydrated in water and washed with binding buffer. 4× volume of the poly (I) solution was added and incubated ON to make poly (I:C) beads. Pulldowns with poly (I:C)- or poly (C)-agarose were performed with lysates from HEK293T cells that have been transfected with either RIG-I or DDX3 in the presence of RNAse inhibitors. Pulldowns were then analysed by SDS-PAGE and western blotting.

### Alignments of K7R

The alignment of K7R and A52R was performed using ClustalW software and edited with Genedoc. The alignment and phylogenetic tree of the K7R orthologs was performed using CLC Free Workbench 2.

### Results

K7 is a member of a family of VACV proteins that includes A52 (H. Smith et al. 1991), a TLR-antagonist. A52 (VACV_WR178) is a 190 amino acid (aa) protein, while K7 (VACV_WR039) is only 149 aa long. These proteins are each acidic (pls 5.45 and 4.75 for A52 and K7, respectively) and lack predicted transmembrane sequences. An alignment of A52 and K7 from the VACV strain Western Reserve (WR) shows 25% aa identity and 50% similarity (FIG. 1A). Analysis of the conservation of A52 and K7 in VACV orthopoxviruses using available sequence data at www.poxvirus.org shows that K7 is more conserved between different viruses and shares greater than 95% aa identity between all orthopoxvirus orthologues (FIG. 1B). This high degree of conservation suggests an important function for K7.

K7 was cloned from genomic DNA of VACV WR (VACV-WR_{—}039) into a mammalian expression vector containing an HA-epitope tag for immunodetection. When increasing amounts of this plasmid vector were transfected into HEK293T cells, a concomitant increase in K7-HA expression at its predicted molecular mass of 17.5 kDa was detected (FIG. 1C).

A polyclonal antiserum against K7 was generated by immunising rabbits with recombinant K7 that was produced in E. coli (for details see Materials and Methods). This antibody was then used to detect K7 in VACV WR-infected HEK293 cells. Expression of K7 was visible as early as 2 hours post-infection (p.i.) and increased until 24 hours p.i. (FIG. 1D).

The presence of cytosine β-D-arabinofuranoside (AraC), an inhibitor of poxvirus DNA replication and thereby of intermediate and late gene expression, reduced, but did not ablate, K7 expression at 24 hours (data not shown). Hence, K7 is expressed both early and late during infection.

To test experimentally if K7 was expressed by different orthopoxviruses, extracts from cells infected with different VACV or CPXV strains were immunoblotted with anti-K7 Ab. K7 was expressed by all 16 VACV strains and 2 CPXV strains (Brighton Red and elephantpox virus) tested, in accordance with its high degree of conservation within this family (FIG. 1E, lower panel).

K7 was tested to see if it inhibited NF-κB activation induced by IL-1/TLR signalling. The IL-1 receptor is part of the TLR receptor family and utilises MyD88 as its sole adaptor for signalling to NF-κB activation. K7 expression inhibited IL-1-induced activation of the NF-κB reporter, and the IL-8 promoter reporter (an NF-κB-dependent gene), in a dose-dependent manner (FIG. 2A).

Also like A52, expression of K7 inhibited NF-κB activation induced by TLR4. FIG. 2B shows that K7 expression inhibited NF-κB activation induced by the constitutively active CD4-TLR4 or any of the four adaptor molecules utilised by TLR4. Similarly, NF-κB activation by TLR3 (induced by the synthetic dsRNA analogue poly(I:C)) was also inhibited. (FIG. 2C). It was noted, that K7 expression also led to a lower basal activation level (see second column). To determine whether K7-mediated inhibition of NF-κB had downstream functional consequences, NF-κB-dependent cytokine expression was examined and it was found that K7 inhibited LPS- or MyD88-induced IL-8 production as well as poly(I:C)— or TRIF-induced RANTES production (FIG. 2D).

### Example 1

### K7 Interacts with the A52 Targets IRAK2 and TRAF6

K7 displayed very similar effects to A52, and therefore it was investigated whether K7 bound to the same intracellular targets as A52, namely IRAK2 and TRAF6. K7 co-immunoprecipitated with IRAK2 when both proteins were co-expressed in HEK293T cells (FIG. 3A, upper panel, lanes 6 and 9). Similarly, K7 co-immunoprecipitated with TRAF6 (FIG. 3B, upper panel, lane 3), but did not interact with TRAFs 1-5 (FIG. 3B, upper panel, lanes 6, 9, 12, 15, 18) and therefore targets TRAF6 specifically.

In order to characterise the interaction with TRAF6 further, we showed that K7 co-immunoprecipitated with the TRAF domain of TRAF6 (FIG. 3C, upper panel, lane 3), the domain of TRAF6 that A52 binds to. Previously, we had demonstrated that a truncated mutant of A52, ΔA52, containing aa 1-144, still binds to IRAK2 but fails to interact with TRAF6. Therefore, we constructed truncated mutants of K7 in order to map its interactions with TRAF6 and IRAK2. Surprisingly, a truncation mutant of K7 comprising aa 1-108, corresponding to ΔA52 (compare alignment in FIG. 1A), still interacted with TRAF6. Also, all the other truncated mutants investigated seemed to still interact with both TRAF6 and IRAK2. Thus, K7 may interact with TRAF6 using distinct motifs compared to A52.

Data from this series of co-immunoprecipitation experiments are summarised in FIG. 4. The region between amino acids 41 and 93 of the K7 protein appear to be necessary for the interaction with both TRAF6 and IRAK2.

### Example 2

### Lack of K7 Leads to Strongly Reduced Virulence in Vaccinia Virus Infections

To test the contribution of K7 in VACV virulence, a VACV deletion mutant lacking the K7R gene was constructed (DelK7R) by transient dominant selection. A plaque purified wild type virus (WTK7R) was isolated from the same intermediate virus. As a control, a revertant virus was also constructed by re-inserting the K7R gene into the DelK7R mutant (RevK7R). Restriction digests and PCR analysis confirmed that the genomes of these viruses were as predicted (FIGS. 5A and B). Digestion of the viral genomes with Hind III showed that in DelK7R, but not in control viruses, the K fragment was approximately 0.5 kb smaller, consistent with the deletion of the K7R orf PCR with primers corresponding to sequences flanking the K7R orf gave a product of similarly reduced size, confirming that the deletion is at this locus. In Hind III, Xho I and Nco I (not shown) genomic digests, no other bands were altered in size, showing that no unintentional major genome changes had been made during virus construction. In particular, the Xho I 6.3 kb band was unchanged, showing that the genome termini had not been truncated.

Immunoblotting analysis confirmed expression of K7 in wild-type (WTK7R) and RevK7R, but not in DelK7R (FIG. 5C). A control protein, D8, was expressed at similar levels by all three viruses. The DelK7R virus showed no defect in replication, and all three viruses replicated to the same titres after infection of BS-C-1 cells at 10 plaque forming units (p.f.u.) per cell (FIG. 5E). However, when infected at 0.015 p.f.u. per cell, the DelK7R virus showed slightly lower titres at later time points (48 and 72 hours p.i.) (FIG. 5F). This subtle difference, might be due to either a reduced efficiency of cell to cell spread of virus, or, more probably, due to enhanced cellular defence mechanisms in the absence of K7. The observation that the size of the plaque was similar for all three viruses is consistent with this view (FIG. 5D).

To assess virus virulence, mice were infected intradermally in the ear pinna with either DelK7R, RevK7R or WTK7R and the size of the resulting lesions was measured daily. FIG. 5G shows that mice infected with DelK7R displayed a reduced lesion size compared to WTK7R and RevK7R. These differences were statistically significant over days 7-11, 13 and 14 p.i.

In an intranasal infection model, the difference between the DelK7R and the control viruses was greater (FIG. 5H). DelK7R induced substantially lower weight loss and signs of illness compared to controls between days 4 and 13. Moreover, virus titres in infected lungs were significantly lower after infection with DelK7R at day 6 p.i. compared to controls (FIG. 5I). In addition, at day 6 p.i., in mice infected with DelK7R, there was a significant increase in the number of infiltrating leukocytes (CD45+ cells) in the lung tissue compared to controls (FIG. 5J). Upon closer examination of the cell populations involved, it was found that the increase in CD45+ cells at day 6 in the DelK7R virus correlated with an increase in T cells (FIG. 5K, left panel) and more specifically CD8+ T cells (FIG. 5K, middle panel). This observation is consistent with the concomitant reduction in virus titres, given the importance of T cells in clearing virus infections.

Interestingly, in contrast to T cells, the number of neutrophils was significantly reduced in the lungs of mice infected with the DelK7R virus compared to controls (FIG. 5K, right panel).

### Example 3

### K7 Inhibits NF-κB Activation Induced by Non-TLR Pathways

Deletion of K7R induced a greater attenuation than loss of A52R, suggesting that K7 might have mechanisms to interfere with the immune response additional to those of A52. Therefore, tests were carried out to see if K7 inhibited other signalling pathways important in innate immunity. TNF-α, like IL-1, is an important pro-inflammatory cytokine that exerts many of its effects through NF-κB activation. However, TNF-α uses a distinct set of signalling components, including TRAF2, for the activation of NF-κB. Notably, K7 inhibited TNF-induced activation of the NF-κB reporter, while A52 had no effect (FIG. 6A). Furthermore, although K7 associated with TRAF6 and not TRAF2 (FIG. 3B), it inhibited both TRAF6- and TRAF2-induced NF-κB activation (FIG. 6B).

Another TLR-independent pathway important for the immune response against viruses is elicited by the recognition of dsRNA by the cytoplasmic helicase RIG-I. FIG. 6C shows that K7 can inhibit NF-κB activation induced by RIG-I expression both in the presence and absence of its ligand, the synthetic dsRNA analogue poly(I:C). Because K7 inhibited NF-κB activation by many stimuli acting via different signalling pathways, it was reasoned that K7 must mediate its inhibitory effect downstream from IRAK2 and TRAF6, at a point common to multiple NF-κB activators. Many pathways leading to NF-κB activation utilise the IKK-complex to phosphorylate IκB-A, which leads to its degradation and the release of NF-κB. Over-expression of a key kinase of this complex, IKK-α, leads to NF-κB activation, which was inhibited by K7 but not A52 (FIG. 6D). Therefore, compared to A52, K7 seems to have a different or additional mechanism of interfering with NF-κB activation, which is at, or downstream of, IKK activation. IκB degradation induced by IL-1 was inhibited by both K7 and A52 at 7 min post IL-1 stimulation (FIG. 6E, upper panel, lane 3 and 4). However, the effect of K7 was more transient than that of A52, because at 15 min post stimulation, A52 still inhibited IκB degradation (FIG. 6E, upper panel, lane 6), whereas K7 did not (FIG. 6E, upper panel, lane 7). It is unlikely that this transient inhibition of IκB degradation can fully account for the sustained and potent inhibitory effect of K7 on NF-κB reporter gene assays. This suggests that K7 has additional mechanisms for interfering with NF-κB activation.

### Example 4

### K7, but not A52, Inhibits TLR- and Non-TLR-Dependent Activation of IRFs

In addition to NF-κB activation, another important signalling pathway for induction of anti-viral immunity is the activation of IRF transcription factors, which are essential for the induction of type I IFNs. IRFs are activated by both TLR-dependent and TLR-independent mechanisms (e.g. via RIG-I) during viral infection. It has previously been demonstrated that A52 does not interfere with IRF3 activation induced by TLR3. To investigate the effect of K7 on IRF activation, a transactivation assay was utilised using IRF3 or IRF7 fused to the DNA-binding domain of GAL4 together with a GAL4-dependent reporter. Engagement of TLR3 and TLR4 leads to IRF3 and IRF7 activation via TRIF, while TLR7, 8 and 9 stimulation leads to IRF5 and IRF7 activation via MyD88. Strikingly, K7 but not A52 inhibited both TRIF-induced activation of IRF3 and IRF7 (FIG. 7A) and MyD88-induced activation of IRF7 (FIG. 7B). Thus, K7 is likely to interfere with IFN induction by multiple TLR ligands.

To define the site of action of K7 in the IRF activation pathway, TBK1-induced IRF activation was examined. TBK1 binds to IRF3 and IRF7 and leads to their phosphorylation and activation. Remarkably, K7 inhibited TBK1-induced activation of IRF3 and IRF7 (FIG. 7C) suggesting that K7 acted close to TBK1 or further downstream.

IRF3 and IRF7 can induce promoters containing an IFN-stimulatory response element (ISRE), and consistent with this, K7 inhibited TBK1-induced ISRE activation (FIG. 7D). However, K7 did not affect direct induction of the ISRE by IRF7 expression (FIG. 7D), consistent with K7 inhibiting TBK1 stimulated IRF activation upstream of promoter induction. Inhibition of the IRFs at the level of TBK1 also suggested that K7 would interfere with the RLH pathway, which also utilises TBK1 for IRF activation. Indeed, K7 inhibited ISRE induction mediated by the RLH adaptor MAVS (FIG. 7E).

Sendai virus activation of IRFs and IFN-β induction is mediated by RIG-I, a pathway that also employs TBK1. Consistent with this, K7 (but not A52) inhibited Sendai-virus induced activation of the IFN-β promoter reporter and of IRF7 (FIG. 7F), demonstrating that K7 can interfere with virus-induced IFN induction.

In order to rule out the possibility that K7 expression interferes with some general principle of reporter gene assays, the effect of K7 on an unrelated reporter gene driven by an unrelated stimulus was examined. The protein kinase C (PKC) agonists PMA and ionomycin induced luciferase activity from an AP-1 responsive promoter and this induction was not inhibited by K7 (FIG. 7G).

### Example 5

### K7 Drives p38 Activation and Induces IL-10

In addition to inhibiting TLR-induced NF-κB activation (probably by binding IRAK2), A52 can activate p38 MAP kinase and enhance LPS-induced IL-10 production in a TRAF6-dependent manner. K7R can also interact with TRAF6, and so its effects on p38 activation and IL-10 production was examined. First, p38 activation was assessed using a transactivation assay based on the p38 substrate CHOP fused to the DNA-binding domain of GAL4 in conjunction with a GAL4-dependent reporter construct. Like A52, expression of K7 increased reporter expression in this assay (FIG. 8A). IL-10 is an NF-B-independent gene but is regulated by MAP kinases. In the murine macrophage cell line RAW264.7, we found that transfection of K7, but not A52, was sufficient to drive IL-10 production (FIG. 8B). A52 was capable of enhancing IL-10 production only in the presence of LPS (FIG. 8B), as shown previously. Thus, although K7 leads to a similar level of p38 activation compared to A52, it has more potent effects on IL-10 production, being capable of inducing IL-10 production in the absence of additional stimulation.

### Example 6

### K7, but not A52, is Localised in the Nucleus

When K7 was fused to the GAL4-DNA binding domain of a Yeast-2-hybrid bait vector it auto-activated yeast reporters (data not shown). Therefore, it was postulated whether K7 could enter the nucleus and act proximal to specific promoters. To investigate their intracellular localisation, A52 and K7 were fused to EYFP (enhanced yellow fluorescent protein) and the sub-cellular location of the fusion proteins assessed by confocal microscopy (FIG. 9). K7-EYFP was present in the nucleus and cytoplasm whereas A52 fused to EYFP was cytoplasmic. A similar distribution was found using HA-tagged K7 and staining with an AlexaFluor594-labelled anti-HA antibody (data not shown). Interestingly, A52-EYFP showed a very different distribution and was excluded from the nucleus. Stimulation of the cells with IL-1 or TNF did not change the localisation of either protein (data not shown). The finding that K7 but not A52 enters the nucleus supports our hypothesis that K7 has additional mechanisms for modulating gene expression.

### Example 7

### K7 Targets the Cellular DEAD-Box Helicase DDX3

Data presented hitherto show that K7 mimics some of the effects of A52 and shares some of its targets (TRAF6 and IRAK2). However K7 also inhibits IRF activation and affects NF-κB activation and IL-10 induction more extensively than A52. Therefore, it was attempted to identify further targets of K7 that might be involved in mediating these additional effects.

The Yeast-2-hybrid system could not be employed because K7 auto-activated the reporters used when it was fused to the DNA-binding domain of GAL4 (data not show). Therefore, recombinant His-tagged K7 was used to pull down interacting proteins from HEK293 cell lysates and these were analysed by SDS-PAGE (FIG. 1A). In a one-dimensional Coomassie stained gel, a band of approximately 70 kDa was identified that appeared reproducibly in the K7R pulldown lane but not in the control lane (FIG. 10A). This band was identified by MALDI-TOF as DDX3 (also called DBX or Cap-Rf), a DEAD-box-containing putative RNA helicase of 73 kDa. When the pulldown experiment was repeated (with His-tagged Rab6 as an unrelated control protein) and western blot analysis performed using a polyclonal antiserum against DDX3, a band of the right molecular weight was detected in the K7R but not in the control pulldowns (FIG. 10B). In order to confirm the K7-DDX3 interaction by an alternative method, HA-tagged K7 was transfected into HEK293T cells in order to perform co-immunoprecipitation experiments. When K7R-Ha was immunoprecipitated with an antibody against the Ha-tag, endogenous DDX3 co-precipitated with it (FIG. 10C). To establish whether this interaction would occur in VACV-infected cells, HEK293 cells were infected with VACV WR and K7 immunoprecipitated with the K7-specific antiserum. Endogenous DDX3 co-precipitated with K7 produced during VACV infection (FIG. 10D), confirming that this interaction occurs with physiological levels of both proteins.

The aim of these pulldown experiments was to identify targets of K7 that would not interact with A52. Accordingly, K7-HA or A52 were expressed in HEK293T cells and immunoprecipitated with anti HA mAb or A52-specific antiserum, respectively. DDX3 co-precipitated with K7 but not with A52 (FIG. 10E, upper panel, lane 2 and 4), confirming that DDX3 is targeted by K7R but not by A52R.

The recognition of dsRNA in the cytoplasm is mediated by an RNA helicase, the DExD/H box protein RIG-I. Since K7 inhibits RIG-1-mediated NF-κB activation and in DDX3 interacts with an RNA helicase, it was suggested that K7 might also interact with RIG-I. However, no interaction between these K7 and RIG-I was detected in co-immunoprecipitations with Flag-RIG-I and K7-HA (FIG. 10F).

### Example 8

### K7 can Interact with DDX3 in Cytoplasm and Nucleus

It has been reported that DDX3 shuttles between the cytoplasm and the nucleus, being exported from the nucleus via the CRM-1 system. K7 is also localised in both cytoplasm and nucleus, and so it was postulated whether the two proteins would interact with each other in the cytoplasm or the nucleus or both. DDX3 shuttling between cytoplasm and nucleus via CRM-1 was confirmed by immunoblot analysis of sub-cellular fractions. In the absence of the CRM-1 inhibitor leptomycin B (LMB), DDX3 was detected mostly in the cytoplasmic fraction, while treatment with LMB led to its accumulation in the nucleus (FIG. 11A). This result was confirmed by immunofluorescent staining and confocal microscopy. In the absence of LMB, DDX3 is located in the cytoplasm and can not be detected in the nucleus with this method. However, four hours after the addition of LMB, DDX3 had accumulated in the nucleus (FIG. 11B). It was then attempted to determine the localisation of K7 and DDX3 within the cell by confocal microscopy. In the absence of LMB, K7 and DDX3 co-localised in distinct spots in the cytoplasm. After treatment with LMB, DDX3 moved to the nucleus and co-localised with nuclear K7. As a control, A52-EYFP and DDX3 were co-expressed and no co-localisation of A52 and DDX3 was found (FIG. 11C).

These results suggest that K7 and DDX3 can interact either in the cytoplasm or the nucleus. Therefore, it was postulated whether the functions of K7 would depend on its nuclear or cytoplasmic interaction with DDX3 or whether it needed DDX3 to shuttle between the two compartments. FIG. 11D shows that in the presence of LMB, K7 was still capable of inhibiting Sendai virus-induced activation of the IFN-β promoter, and IL-1-induced activation of the IL-8 promoter (FIG. 11D).

### Example 9

### K7 binds to the N-Terminal Region of DDX3

The region of K7 needed for the interaction with DDX3 was studied using the truncated mutants of K7 described above and generated truncations of DDX3. The C-terminal region of DDX3 (amino acids (aa) 409-662) was described to act as a dominant negative mutant of DDX3 function and to bind to the HCV core protein. Therefore, His-tagged full length DDX3 (amino acids 1-662) and mutants containing amino acids 409-662, amino acids 1-408, amino acids 22-408, amino acids 102-408 and amino acids 139-408 were constructed (FIG. 12C). These were used in pull down experiments using cell lysates containing overexpressed K7-HA or AK7-HA (1-108). It was found that full-length K7 was pulled down with full-length His-DDX3, DDX3 1-408 and DDX3 22-408, but not with DDX3 409-662, DDX3 102-408 or DDX3 139-408 (FIG. 12A, top panel). Moreover, K7 amino acids 1-108 was not pulled down by any of the DDX3-His constructs in a parallel experiment (FIG. 12A, bottom panel) indicating amino acids 108-149 of K7 are needed to bind DDX3. This result was confirmed in immunoprecipitation experiments with Myc-tagged DDX3 1-408 (the region of DDX3 that full-length K7 interacted with in the His-pulldowns) and ΔK7-HA. This experiment also failed to show an interaction between ΔK7R and DDX3 (FIG. 12B, right panel) while the interaction of full-length K7 with DDX3 1-408 was confirmed (FIG. 12B, left panel).

The results of this series of immunoprecipitation experiments with the Myc-tagged truncations of DDX3 are summarised and depicted in a schematic (FIG. 12C). These data suggest that K7 interacts with DDX3 between aa 22 and 102.

### Example 10

### K7 does not Interfere with Substrate Binding of DDX3

The binding of K7 to the region of DDX3 containing the DEAD-box and the Ib (TPGR)-motif, which is involved in binding of the substrate RNA, suggested that K7 might displace the substrate RNA and so this was examined experimentally. The substrate specificity of DDX3 was tested using pulldown assays with poly (C) and poly (I:C)-coated agarose beads, representing ssRNA and dsRNA substrates, respectively. In this assay, DDX3 bound both ssRNA and dsRNA substrates (FIG. 12D, upper left panel). As a control, it was demonstrated that RIG-I, the DexD/H-box helicase involved in the recognition of cytoplasmic dsRNA, bound only dsRNA as described previously (FIG. 12D, lower left panel). The presence of K7 did not interfere with substrate binding of DDX3 in this assay (FIG. 12D, upper right panel).

### Example 11

### The C- and N-Terminal Regions of K7 Mediate the Interaction with DDX3

To map the region of K7 needed for the interaction with DDX3 was investigated by immunoprecipitations with DDX3-Myc and HA-tagged truncated mutants of K7. The results of these experiments are summarised in FIG. 12E. DDX3 interacted with full-length K7 but failed to interact with any of the C-terminal truncation mutants 1-93, 1-108 (ΔK7) and 1-123 and the N-terminal truncation mutant K7 41-149. These results suggest that the C terminus (aa 123 to 149) and the N-terminus (1-41) of K7 are needed to bind DDX3. It was also shown that the interaction with TRAF6 and IRAK2 maps to a region between aa 41 and 93 of K7R. Therefore, ΔK7 (aa 1-108) fails to bind to DDX3 but still interacts with TRAF6 and IRAK2.

### Example 12

### ΔK7 Still Enters the Nucleus but Looses its Inhibitory Function

Next, the functional activity of ΔK7 and K7 were compared in order to determine the importance of the interaction with DDX3 for K7R function. Signalling pathways affected by K7 but not by A52 were examined first. FIG. 13A shows that ΔK7 failed to inhibit TBK1-induced IRF3, IRF7 and IFN-β activation while full-length K7 inhibited these signals. Similarly, another TLR-independent pathway, activation of the NF-κB reporter by RIG-I, was inhibited by K7 but not ΔK7 (FIG. 13B). However, when we examined TLR-dependent signalling pathways, such as IL-1 induced NF-κB activation or TRIF-induced IFN-β promoter activation, we found that ΔK7R also failed to show an effect on these pathways (data not shown). Therefore, a K7 mutant, that can still bind to TRAF6 and IRAK2 but fails to interact with DDX3, lost its ability to block TLR-dependent and -independent pathways. However, both ΔK7 and full length K7 stimulated p38 MAP kinase activation to a comparable level (FIG. 13C) (probably mediated by the interaction with TRAF6 (19)) suggesting that the failure of ΔK7 to inhibit other pathways is not due to improper folding and/or degradation of the truncated protein, but that the DDX3-binding C-terminus of K7 is crucial for the inhibitory effect of K7. Thus the ability of K7 to inhibit IRF activation correlated with binding to DDX3. Lastly, we investigated whether the lack of inhibitory activity was due to a failure of ΔK7 to enter the nucleus. However, EYFP-tagged ΔK7 still appeared in the nucleus (FIG. 13D). Thus, the interaction with DDX3 is not needed for K7 to enter the nucleus. Also, even though the nuclear location is a difference between K7 and A52, the presence of K7 in the nucleus is not sufficient for it to exert its inhibitory effects.

### Example 13

### DDX3 is a Positive Effector of the IRF Pathway

The above observations suggested that targeting of DDX3 by K7 mediated the inhibitory effects of K7 on IRF activation, and therefore the role of DDX3 in the IRF pathway was investigated further. Previously, HIV was shown to exploit DDX3 CRM-1-dependent shuttling between the cytoplasm and the nucleus to export its mRNAs from the nucleus. In that study, a truncated form of DDX3, containing just the C terminus (aa 408-662) acted as a dominant negative mutant of endogenous DDX3 function in so far as it related to enhancement of HIV gene expression. To determine whether DDX3 is directly involved in IKK-E and TBK1-mediated induction of the IFN-β promoter, the ability of this dominant negative DDX3 mutant (dDDX3) to inhibit promoter induction was assessed. FIG. 14A shows that dDDX3 inhibited TBK1-induced IFN-β promoter activity, while it had a more modest effect on IKKε-mediated promoter induction (FIG. 14B). Consistent with these data, expression of wild-type DDX3 enhanced both TBK1- and IKKε-mediated promoter induction (FIG. 14A, B). Of note, the potency of inhibition by dDDX3 on TBK1 versus IKKε correlated with that seen for K7 (FIG. 14A, B). Furthermore, expression of wild type DDX3 protein alone was sufficient for IRF7 activation (FIG. 14C), while having no effect on IRF7-mediated induction of the ISRE (FIG. 14D). These data place DDX3 downstream of TBK1-stimulated IRF activation but upstream of promoter induction (FIG. 16). This correlates directly with the point of inhibition of the IRF pathway by K7 (FIG. 7C, D). The identification of DDX3 as a target for K7, together with the above observations, suggests a novel role for DDX3 in innate immune signalling to IRF activation.

### Example 14

DDX3 is a positive effector of NF-κB activation. Apart from a role in IRF activation, FIG. 15 presents evidence that DDX3 may also be important for NF-κB activation. It is well-known that IκB kinases (IKKs) are crucial to NFκB activation, especially IKKα and IKKβ, and that both IκB and NFκB are substrates for these kinases. Other proteins that contribute to NFκB activation, such as IKKγ or TAK1 are known to interact with the IKKs. FIG. 15A shows that when a plasmid expressing Myc-tagged DDX3 was transfected into HEK293 cells together with a plasmid expressing either Flag-tagged IKKα (lanes 1 and 3), IKKβ (lanes 4 and 6) or IKKε (lanes 7 and 9), DDX3 could be immunoprecipitated in a complex with the aforementioned IKKs (seen in lanes 3, 6 and 9). Thus DDX3 may be important in IKK-mediated NF-κB activation. It is apparent from FIG. 15A, lower panels, that the presence of overexpressed IKKs affects the expression profile of DDX3, in that a slower migrating form of DDX3 was observed (lanes 3, 6 and 9). This is shown more clearly in FIG. 15B, and labelled *DDX3. FIG. 15B shows that expression of IKKε caused the appearance of a slower migrating form of DDX3, and that expression of K7 inhibited the appearance of *DDX3. Treatment with phosphatase demonstrated that *DDX3 is a phosphorylated form of DDX3 (data not shown). Therefore, IKKs can likely phosphorylate DDX3, as well as NF-κB. A further link between DDX3 and NF-κB is shown in FIG. 15C where DDX3 is shown to associate with the NF-κB subunit p65, since when HA-p65 was immunoprecipitated, Myc-DDX3 could be detected by immunoblot (H.C.=antibody heavy chain). These interactions of DDX3 with the IKKs and NF-κB are likely to have functional relevance since expression of DDX3 can also stimulate activation of NF-κB subunits and NF-κB-dependent reporter genes, as shown in FIG. 15D-F. FIG. 15D shows a p65 transactivation reporter gene assay which measures the ability of expressed proteins to activate the NF-κB subunit p65. As shown, when 0-150 ng of a plasmid encoding DDX3 was transfected into cells containing the p65-Gal4 and pFR luciferase plasmids, dose-dependent activation of p65 was achieved. In a similar assay to measure activation of the NF-κB subunit p52, expression of DDX3 (25-100 ng of plasmid) led to dose-dependent activation of p52 (FIG. 15E). Furthermore, transfection of 25 or 50 ng of the DDX3-expressing plasmid significantly augmented p65-induced p52 activation (FIG. 15E, last three columns). Since both IKKε and p65 are required to stimulate p52 transactivation, DDX3 could conceivably facilitate p52 transactivation via its interaction with either IKKε (FIG. 15A) or p65 (FIG. 15C). Finally, FIG. 15F shows that DDX3 expression (from 0-100 ng of plasmid), either alone, or in combination with IKKα or IKKβ leads to activation of an NF-κB-dependent reporter gene.

### Discussion

The examples show the effects of the two proteins A52 and K7 from vaccinia virus that share significant sequence similarity. Even though they are both immunomodulators and can target similar signalling pathways, they have a different specificity and potency. A52 specifically inhibits TLR-induced NF-κB activation, which seems to be mostly mediated by its interaction with IRAK2. K7 can also bind to IRAK2 and inhibits TLR-induced NF-κB activation, however it also potently inhibits NF-κB activation induced by non-TLR stimuli as well as basal activation levels. Therefore, compared to A52, K7 has evolved an additional mechanism to target the NF-κB pathway further downstream. Without being bound by theory, we predict that this mechanism may act at the level of IκB-phosphorylation and degradation. Other possibilities would be that it inhibits p65 phosphorylation, blocks nuclear translocation or interferes with binding of NF-κB to the promoter or the transactivation of the promoter. Further studies addressing this additional mechanism are currently underway.

K7 can block the activation of IRFs, which are crucial for the induction of type I interferons, mediators with potent anti-viral properties. Similar to the inhibition of the NF-κB pathway, K7 has a broad inhibitory effect on IRF activation, extending to TLR- and non-TLR dependent pathways. TBK1 is the kinase directly mediating the phosphorylation and activation of the IRFs. Therefore, the fact that K7 inhibits TBK1-induced IRF activation points to a mechanism that either directly interferes with this activation step or acts even further downstream. Neither the inhibitory effect on NF-κKB activation by non-TLR pathways nor the inhibition of IRF activation can be explained by the targeting of IRAK2 or TRAF6, since neither molecule is involved in these pathways. Further, the presence of K7 in the nucleus might suggest that it exerts its additional effects in the nucleus proximal to the promoter of the genes regulated.

The third target of K7, the DEAD-box helicase DDX3, has been described to constantly shuttle between the nucleus and the cytoplasm. K7 doesn't seem to change the distribution of DDX3 between the two compartments. It has been shown that DDX3 and K7 could co-localise in the cytoplasmic as well as in the nuclear compartment. However, in the absence of the CRM-1 inhibitor Leptomycin B, DDX3 was not detectable in the nucleus by our immunofluorescent staining technique. DDX3 levels in the nucleus are kept low by its constant export from the nucleus via the CRM-1 export system. Because of that, DDX3 co-localising with K7 in the nucleus in the absence of leptomycin B was not detected, but it is conceivable that just a few DDX3-K7 complexes in the nucleus might be sufficient for an effect.

Without being bound by theory, it was predicted that the function of K7 is also unlikely to depend on the export of DDX3 from the nucleus, since K7 retains its inhibitory function in the presence of the CRM-1 inhibitor, which prevents export of DDX3. On the other hand, the import of K7 also did not depend on its interaction with DDX3, because the ΔK7 mutant that fails to bind DDX3 still localises to the nucleus. Therefore, it is unlikely that K7 is exploiting the transport function of DDX3. DEAD box helicases are involved in all processes involving RNA, such as splicing, mRNA transport and translation. It has also been shown that DEAD-box proteins can interact with the transcription machinery. For DDX3 in particular, it has been implicated in regulation of translation and cell growth. However, it is unlikely that K7 interferes with fundamental cellular processes or protein translation in general, because K7 does not shut down gene expression in general, nor does it inhibit an AP-1 reporter gene, but activates p38 and induces IL-10 production.

The interaction with DDX3 seems to be crucial for the inhibitory action of K7, since a truncated version of K7 that fails to bind DDX3 also looses its inhibitory function. This truncated K7 (ΔK7) inhibits neither TLR-dependent nor TLR-independent signalling to NF-κB or IRFs. p38 can be equally well activated by the full-length K7. This finding excludes a loss of function due to misfolding of the truncated protein and implies that DDX3 is not involved in mediating the stimulatory effects of K7. Activation of p38 by A52 is mediated by its interaction with TRAF6 suggesting that this might also be the case for K7. It is surprising that ΔK7 does not mimic the effect of A52 on TLR-induced NF-κB activation given that it can still interact with IRAK2 (not shown), which mediates the inhibitory effects of A52. However, the interaction that is observed between K7 and IRAK2 appears quite weak compared to the interactions with TRAF6 and especially DDX3.

Also, since the inventors found that TRAF6 and IRAK2 binding map to the same region of K7 (which is different to A52 where TRAF6 and IRAK2 binding can be clearly attributed to different regions of A52) it is possible that IRAK2 does not directly interact with K7 but rather indirectly via TRAF6. Alternatively, although ΔK7 still binds to IRAK2, it may lack residues required to inhibit IRAK2 function.

Furthermore, in VACV infected cells the interaction with DDX3 can be detected, but an interaction between K7 and TRAF6 or IRAK2 (data not shown) cannot be seen. It is therefore possible that K7R, even though it retains the ability to interact with TRAF6 and IRAK2 when these proteins are over-expressed, does not actually target them with functional consequences. These interactions might be an evolutionary rudiment while K7 has evolved new mechanisms of interfering with gene expression in a broader fashion downstream of TRAF6 and IRAK2 and not limited to TLR pathways. The C-terminus of K7 (aa 108-149) seems to mediate the inhibitory action of K7, probably through the interaction with DDX3. The possibility that this region of K7 binds additional targets which mediate the inhibitory action cannot be excluded.

K7, as opposed to A52, is localised mainly in the nucleus. Therefore K7 could exert its effects very proximal to the promoter level. K7 when fused to the GAL4 DNA-binding domain would auto-activate the reporters in a Yeast-2-hybrid assay. This has also previously been previously described. Auto-activation in this set-up generally means that the protein in question contains a trans-activation domain. GAL4 itself contains an acidic activation domain. K7 is a very acidic protein and might therefore be able to assume the function of an acidic activation domain. Activation domains act by recruiting the general transcription machinery to the promoter of the gene. It is possible that K7 acts as a ‘decoy trans-activation domain’ by sequestering an important component of the transcription machinery, and thereby inhibiting the expression of NF-κB and IRF-dependent genes. If brought into close proximity with a specific promoter (such as the IL-10 promoter) it might then through the same mechanism be able to trans-activate the transcription of this promoter.

K7 is a very potent immunomodulatory protein with a broader range of inhibition than the related A52 protein. This is also reflected by the in vivo phenotype of the vaccinia virus lacking K7R. This virus showed severely reduced virulence in intranasal and intradermal infection models, which was a much stronger phenotype than the one observed by the vaccinia virus lacking A52R. However, despite their sequence similarity and the broader action range of K7, both A52 and K7 independently contributed to virulence. A rationale for the major contribution of K7 to virulence is provided by the fact that we have shown that K7 targets most of the known anti-viral signalling pathways leading to NF-κB and IRF activation, the key transcription factors that programme the host anti-viral innate response. In addition, K7 directly induces IL-10 release from cells. Viruses are known to increase IL-10 in a host as an immune escape mechanism. Unlike other viruses such as EBV and poxvirus Orf, VACV does not encode an IL-10 homologue. However, VACV replication has been shown to be impaired in IL-10^{-/-} mice, thus suggesting that VACV would directly benefit from K7-induced IL-10.

Apart from the fact that K7 contributes strongly to virulence and targets DDX3, the inventors have provided several lines of evidence for a role for DDX3 in innate immune signalling to IRF activation. Firstly, the ability of K7 to inhibit IRFs correlated with binding to DDX3, since a C-terminally truncated version of K7 (ΔK7) that failed to bind DDX3 also lost its ability to inhibit IRF activation. This was not due to a loss of function of ΔK7 due to misfolding, since this mutant protein could still activate p38 MAP kinase as well as the full-length protein. Activation of p38 by A52 is mediated by its interaction with TRAF6 suggesting that this might also be the case for K7. Secondly, a truncated form of DDX3 acted as a dominant negative against endogenous DDX3 and strongly suppressed TBK1-induced IFNβ promoter, while over-expression of wild type DDX3 protein enhanced promoter induction. Thirdly, the ability of dominant negative DDX3 to inhibit promoter induction by TBK1 or IKKε correlated with the level of inhibition observed with K7. Fourth, DDX3 expression was sufficient to activate IRF7. Fifth, DDX3 was placed downstream of TBK1-stimulated IRF activation but upstream of promoter induction, which directly correlated with the point of inhibition of the IRF pathway by K7. Thus, the inventors have identified DDX3 as a novel positive regulator of innate immune signalling pathways to IFN induction, acting downstream of TBK1 to facilitate IRF activation.

DEAD box helicases have been implicated in transcriptional regulation and DDX3 in particular drives the p21 promoter and interacts with the Sp1 transcription factor. Thus DDX3 may be involved in the activation of a number of transcription factors. Further studies will be necessary to determine the exact mechanism by which DDX3 regulates the IRF pathway. Interestingly, DDX3 expression is upregulated in an IRF7-dependent manner, an observation that provides another link between DDX3 and the IRF pathway. The role of DDX3 in IRF activation may also provide a rationale as to why HCV core protein targets DDX3, since this may be a HCV mechanism for suppressing IFN induction.

The importance of DDX3 in innate immunity is likely underscored by the role of K7 in virus virulence. Mice infected intranasally with DelK7R showed only slight signs of illness and had significantly lower viral titres in the lung compared with both control viruses by day 6. Further, DelK7R elicits an enhanced T cell response in infected lungs and is therefore cleared more rapidly from infected mice. This phenotype was stronger than that observed with a VACV mutant lacking A52R where a milder attenuation was observed in the intranasal model, and no attenuation was seen in the intradermal. This suggests that targeting of DDX3 by K7 is physiologically important for VACV. VACV triggers both TLR-dependent and TLR-independent pathways in DCs leading to the induction of pro-inflammatory cytokines and IFN-β respectively, and both pathways were required to elicit activation of innate and adaptive immunity against the virus. These findings emphasise the relevance of K7's ability to disable both TLR-dependent and TLR-independent IFN induction. Interestingly, the newly described cytoplasmic DNA sensor DAI is able to detect VACV DNA and also recruits TBK1 during IFN induction, so it can be predicted that K7 would also inhibit this aspect of the immune response. K7 is also expressed by the MVA strain that is commonly used as a vaccine vector. Since a virus lacking K7 elicits fewer signs of illness in infected mice as well as a stronger immune response, the present findings have implications for the design of safer and/or more efficient vaccines. DDX3 has been postulated as a potentially interesting drug target and the present study should further the interest in DDX3 by drawing attention to an important novel role of this protein in innate immunity.

In conclusion, the inventors have now identified a new role for DDX3 in immune signalling. The inventors have shown that expression of DDX3 leads to NF-κB and IRF activation while dominant-negative DDX3 mimics the effect of K7. Furthermore, the inventors have identified the modulation of DDX3 as a mechanism to suppress or downregulate an aberrant immune response, such as that associated with an autoimmune disease. In K7, the inventors have identified a multifunctional VACV virulence factor that targets DDX3 and may therefore be used in the treatment of diseases caused by an aberrant immune response.

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention. Reference to any prior art in this specification is not, and should not be taken as, an acknowledgment or any form of suggestion that this prior art forms part of the common general knowledge in any country.

1. A method for the treatment and/or prophylaxis of a viral infection or a condition mediated by a pro-inflammatory immune response, the method comprising the steps of:
providing a therapeutically effective amount of a compound which inhibits the expression or biological function of a DEAD-box protein DDX3 comprising the amino acid sequence of SEQ ID NO:1, and
administering the same to a subject in need of such treatment.

2. (canceled)

3. The method as claimed in claim 1 wherein the compound is a polypeptide comprising the amino acid sequence of SEQ ID NO:3 or a fragment thereof.

4. The method as claimed in claim 1 wherein the compound is a polynucleotide which encodes a polypeptide having the amino acid sequence of SEQ ID NO:3 or a fragment thereof.

5. The method as claimed in claim 1 wherein the compound is a polynucleotide comprising the sequence of SEQ ID NO:4.

6.-7. (canceled)

8. The method as claimed in claim 1 wherein the condition is an autoimmune disease.

9. The method as claimed in claim 8 wherein the autoimmune diseases is selected from the group consisting of multiple sclerosis, rheumatoid arthritis, Crohn's disease, psoriasis, systemic lupus erythematosis (SLE), lupus, type I diabetes, colitis, inflammatory bowel disease, asthma and allergy.

10. A pharmaceutical composition for suppressing a viral infection or a pro-inflammatory immune response, comprising a therapeutically effective amount of a compound which inhibits the function or expression of the DEAD-box protein DDX3 having the amino acid sequence of SEQ ID NO:1 along with a pharmaceutically acceptable diluent, excipient or carrier.

11. A pharmaceutical composition as claimed in claim 10 wherein the DDX3 inhibitory compound is a polypeptide comprising the amino acid sequence of SEQ ID NO:3 or an analogue, derivative, fragment, variant or peptidomimetic thereof.

12. A pharmaceutical composition as claimed in claim 10 wherein the DDX3 inhibitory compound is a polynucleotide which encodes a polypeptide having the amino acid sequence of SEQ ID NO:3 or an analogue, derivative, fragment, or variant thereof.

13. A pharmaceutical composition as claimed in claim 10 wherein the DDX3 inhibitory compound is a polynucleotide comprising the sequence of SEQ ID NO:4.

14.-20. (canceled)

21. A method of suppressing an intracellular signalling pathway wherein said signalling pathway activates at least one Interferon Regulatory Factor (IRF) transcription factor, said method comprising the steps of:
providing a therapeutically effective amount of a compound which inhibits the expression or biological function of a DEAD-box protein DDX3 having the amino acid sequence of SEQ ID NO:1, and
administering the same to a subject in need of such treatment.

22. The method of claim 21 wherein the intracellular signalling pathway is induced by an activated Toll-like Receptor, wherein the Toll-like Receptor is activated following the binding of a pathogen-associated molecular pattern (PAMP) to the Toll-like Receptor.

23. (canceled)

24. The method of claim 21 wherein the at least one IRF is IRF3 or IRF7.

25. The method as claimed in claim 21 wherein the DDX3 inhibitory compound comprises the amino acid sequence of SEQ ID NO:3 or a fragment thereof.

26. The method as claimed in claim 21 wherein the DDX3 inhibitory compound is a polynucleotide which encodes a polypeptide having the amino acid sequence of SEQ ID NO:3 or a fragment thereof.

27. The method as claimed in claim 21 wherein the DDX3 inhibitory compound is a polynucleotide comprising the sequence of SEQ ID NO:4.

28.-46. (canceled)

47. A recombinant poxvirus according to claim 46 wherein the genome of the poxvirus lacks the coding sequence of the K7R gene.

48.-49. (canceled)

50. A recombinant poxvirus as claimed in claim 47 wherein the poxvirus further comprises within its genome, at least one non-poxvirus gene or a fragment of a non-poxvirus gene which gene or fragment encodes an antigen or a fragment thereof.

51.-54. (canceled)

55. The method as claimed in claim 1, wherein the viral infection is HIV.

56. The method as claimed in claim 1, wherein the viral infection is Hepatitis C virus.


SEQUENCE LISTING (22 sequences)

SEQ ID NO: 1 - protein, 662 aa, Homo sapiens
MSHVAVENALGLDQQFAGLDLNSSDNQSGGSTASKGRYIPPHLRNREATKGFYDKDSSGWSSSKDKDAYSSFGSRSDSRGKSSFFSDRGSGSRGRFDDRGRSDYDGIGSRGDRSGFGKFERGGNSRWCDKSDEDDWSKPLPPSERLEQELFSGGNTGINFEKYDDIPVEATGNNCPPHIESFSDVEMGEIIMGNIELTRYTRPTPVQKHAIPIIKEKRDLMACAQTGSGKTAAFLLPILSQIYSDGPGEALRAMKENGRYGRRKQYPISLVLAPTRELAVQIYEEARKFSYRSRVRPCVVYGGADIGQQIRDLERGCHLLVATPGRLVDMMERGKIGLDFCKYLVLDEADRMLDMGFEPQIRRIVEQDTMPPKGVRHTMMFSATFPKEIQMLARDFLDEYIFLAVGRVGSTSENITQKVVWVEESDKRSFLLDLLNATGKDSLTLVFVETKKGADSLEDFLYHEGYACTSIHGDRSQRDREEALHQFRSGKSPILVATAVAARGLDISNVKHVINFDLPSDIEEYVHRIGRTGRVGNLGLATSFFNERNINITKDLLDLLVEAKQEVPSWLENMAYEHHYKGSSRGRSKSSRFSGGFGARDYRQSSGASSSSFSSSRASSSRSGGGGHGSSRGFGGGGYGGFYNSDGYGGNYNSQGVDWWGN

SEQ ID NO: 2 - DNA, 1989 bp, Homo sapiens
atgagtcatgtggcagtggaaaatgcgctcgggctggaccagcagtttgctggcctagacctgaactcttcagataatcagagtggaggaagtacagccagcaaagggcgctatattcctcctcatttaaggaaccgagaagctactaaaggtttctacgataaagacagttcagggtggagttctagcaaagataaggatgcgtatagcagttttggatctcgtagtgattcaagagggaagtctagcttcttcagtgatcgtggaagtggatcaaggggaaggtttgatgatcgtggacggagtgattacgatggcattggcagccgtggtgacagaagtggctttggcaaatttgaacgtggtggaaacagtcgctggtgtgacaaatcagatgaagatgattggtcaaaaccactcccaccaagtgaacgcttggaacaggaactcttttctggaggcaacactgggattaattttgagaaatacgatgacattccagttgaggcaacaggcaacaactgtcctccacatattgaaagtttcagtgatgttgagatgggagaaattatcatgggaaacattgagcttactcgttatactcgcccaactccagtgcaaaagcatgctattcctattatcaaagagaaaagagacttgatggcttgtgcccaaacagggtctggaaaaactgcagcatttctgttgcccatcttgagtcagatttattcagatggtccaggcgaggctttgagggccatgaaggaaaatggaaggtatgggcgccgcaaacaatacccaatctccttggtattagcaccaacgagagagttggcagtacagatctacgaggaagccagaaaattttcataccgatctagagttcgtccttgcgtggtttatggtggtgccgatattggtcagcagattcgagacttggaacgtggatgccatttgttagtagccactccaggacgtctagtggatatgatggaaagaggaaagattggattagacttttgcaaatacttggtgttagatgaagctgatcggatgttggatatggggtttgagcctcagattcgtagaatagtcgaacaagatactatgcctccaaagggtgtccgccacactatgatgtttagtgctacttttcctaaggaaatacagatgctggctcgtgatttcttagatgaatatatcttcttggctgtaggaagagttggctctacctctgaaaacatcacacagaaagtagtttgggtggaagaatcagacaaacggtcatttctgcttgacctcctaaatgcaacaggcaaggattcactgaccttagtgtttgtggagaccaaaaagggtgcagattctctggaggatttcttataccatgaaggatacgcatgtaccagcatccatggagaccgttctcagagggatagagaagaggcccttcaccagttccgctcaggaaaaagcccaattttagtggctacagcagtagcagcaagaggactggacatttcaaatgtgaaacatgttatcaattttgacttgccaagtgatattgaagaatatgtacatcgtattggtcgtacgggacgtgtaggaaaccttggcctggcaacctcattctttaacgagaggaacataaatattactaaggatttgttggatcttcttgttgaagctaaacaagaagtgccgtcttggttagaaaacatggcttatgaacaccactacaagggtagcagtcgtggacgttctaagagtagcagatttagtggagggtttggtgccagagactaccgacaaagtagcggtgccagcagttccagcttcagcagcagccgcgcaagcagcagccgcagtggcggaggtggccacggtagcagcagaggatttggtggaggtggctatggaggcttttacaacagtgatggatatggaggaaattataactcccagggggttgactggtggggtaactga

SEQ ID NO: 3 - protein, 149 aa, Vaccinia virus
MATKLDYEDAVFYFVDDDKICSRDSIIDLIDEYITWRNHVIVFNKDITSCGRLYKELMKFDDVAIRYYGIDKINEIVEAMSEGDHYINFTKVHDQESLFATIGICAKITEHWGYKKISESRFQSLGNITDLMTDDNINILILFLEKKLN

SEQ ID NO: 4 - DNA, 450 bp, Vaccinia virus
atggcgactaaattagattatgaggatgctgttttttactttgtggatgatgataaaatatgtagtcgcgactccatcatcgatctaatagatgaatatattacgtggagaaatcatgttatagtgtttaacaaagatattaccagttgtggaagactgtacaaggaattgatgaagttcgatgatgtcgctatacggtactatggtattgataaaattaatgagattgtcgaagctatgagcgaaggagaccactacatcaattttacaaaagtccatgatcaggaaagtttattcgctaccataggaatatgtgctaaaatcactgaacattggggatacaaaaagatttcagaatctagattccaatcattgggaaacattacagatctgatgaccgacgataatataaacatcttgatactttttctagaaaaaaaattgaattga

SEQ ID NO: 5 - DNA, 32 bp, Artificial
  sense primer for amplification of K7R from genomic DNA of the WR strain of VACV
ccggaattcagatggcgactaaattagattat

SEQ ID NO: 6 - DNA, 31 bp, Artificial
  antisense primer for amplification of K7R from genomic DNA of the WR strain of VACV
acgcgtcgactcaattcaattttttttctag

SEQ ID NO: 7 - DNA, 30 bp, Artificial
  1-93 antisense primer for C-terminal truncation mutant
acgcgtcgactcaatggacttttgtaaaat

SEQ ID NO: 8 - DNA, 31 bp, Artificial
  1-108 antisense primer for C-terminal truncation mutant
acgcgtcgactcagattttagcacatattcc

SEQ ID NO: 9 - DNA, 29 bp, Artificial
  1-123 antisense primer for C-terminal truncation mutant
acgcgtcgactcattggaatctagattct

SEQ ID NO: 10 - DNA, 35 bp, Artificial
  41-149 sense primer for N-terminal truncation mutant
ccggaattcagatgatagtgtttaacaaagatatt

SEQ ID NO: 11 - DNA, 27 bp, Artificial
  sense primer introducing BamHI site
cgcggatccatgtacccatacgatgtt

SEQ ID NO: 12 - DNA, 26 bp, Artificial
  sense primer for introducing EcoRI and SaII restriction sites
gccgaattcggatgagtcatgtggca

SEQ ID NO: 13 - DNA, 25 bp, Artificial
  antisense primer for introducing EcoRI and SaII restriction sites
acgcgtcgactcagttaccccacca

SEQ ID NO: 14 - DNA, 29 bp, Artificial
  antisense primer for cloning DDX3 1-408
acgcgtcgactgaaactcttcctacagcc

SEQ ID NO: 15 - DNA, 30 bp, Artificial
  sense primer for cloning DDX3 409-662
gccgaattcttatgggctctacctctgaaa

SEQ ID NO: 16 - DNA, 31 bp, Artificial
  K7R antisense primer introducing XhoI restriction site
acgcctcgagtccattcaattttttttctag

SEQ ID NO: 17 - DNA, 27 bp, Artificial
  K7R mutant antisense primer introducing XhoI restriction site
acgcctcgaggattttagcacatattc

SEQ ID NO: 18 - DNA, 27 bp, Artificial
  A52R antisense primer introducing XhoI restriction site
acgcctcgagtgacatttccacatata

SEQ ID NO: 19 - DNA, 34 bp, Artificial
  039U primer for generation of recombinant VAVC virus
aacttctagattcaccattacttcttccatgtcc

SEQ ID NO: 20 - DNA, 30 bp, Artificial
  039D primer for generation of recombinant VAVC virus
tgatgaattcggggttgggtgtaagattgg

SEQ ID NO: 21 - DNA, 35 bp, Artificial
  039N primer for generation of recombinant VAVC virus
cccctatatcagactatctcacaaaagacagtagc

SEQ ID NO: 22 - DNA, 34 bp, Artificial
  039C primer for generation of recombinant VAVC virus
gtgagatagtctgatataggggtcttcataacgc